(19) United States
(12) Patent Application Publication
(10) Pub. No.: US 2024/0324852 A1
(43) Pub. Date: Oct. 3, 2024

(54) SYSTEMS AND INTERFACES FOR COMPUTER-BASED INTERNAL BODY STRUCTURE ASSESSMENT

(71) Applicant: Intuitive Surgical Operations, Inc., Sunnyvale, CA (US)

(72) Inventors: Moshe Bouhnik, Holon (IL); Benjamin T. Bongalon, Daly City, CA (US); Emmanuelle Muhlethaler, Tel Aviv-Yafo (IL); Erez Posner, Rehovot (IL); Roee Shibolet, Tel-Aviv (IL); Aniruddha Tamhane, Sunnyvale, CA (US); Adi Zholkover, Givatayim (IL)

(21) Appl. No.: 18/703,590

(22) PCT Filed: Oct. 24, 2022
(86) PCT No.: PCT/US2022/047544

§ 371 Date: Apr. 22, 2024

Related U.S. Application Data
(60) Provisional application No. 63/271,618, filed on Oct. 25, 2021.
(60) Provisional application No. 63/271,629, filed on Oct. 25, 2021.
(60) Provisional application No. 63/271,625, filed on Oct. 25, 2021.

(51) Int. Cl. A61B 1/00; A61B 1/31; A61B 34/00; A61B 34/10; A61B 90/00

(52) U.S. Cl. CPC A61B 1/0005; A61B 1/00009; A61B 34/25; A61B 1/31; A61B 2034/105; A61B 2090/367

(57) ABSTRACT

Various of the disclosed embodiments relate to systems and methods for determining and for presenting surgical examination data of an internal body structure, such as a large intestine. For example, various of the disclosed methods may create a three-dimensional computer model using the examination data and then coordinate playback of the examination data based upon the reviewer's interaction with the model. In some embodiments, the model's rendering may be adjusted to reflect various aspects of the examination, including scoring metrics, identified landmarks, and lacunae in the surgical examination review.

### CROSS-REFERENCE TO RELATED APPLICATIONS

This application claims the benefit of, and priority to, U.S. Provisional Application No. 63/271,618, filed upon Oct. 25, 2021, entitled “COMPUTER-BASED ASSESSMENTS FOR INTERNAL BODY STRUCTURE EXAMINATIONS”, U.S. Provisional Application No. 63/271,629, filed upon Oct. 25, 2021, entitled “SYSTEMS AND INTERFACES FOR COMPUTER-BASED INTERNAL BODY STRUCTURE ASSESSMENT”, and U.S. Provisional Application No. 63/271,625, filed upon Oct. 25, 2021, entitled “COMPUTER-BASED MODEL GENERATION FOR INTERNAL BODY STRUCTURE ASSESSMENT”, each of which are incorporated by reference herein in their entireties for all purposes.

### TECHNICAL FIELD

Various of the disclosed embodiments relate to computer systems and computer-implemented methods for assessing surgical examination of an internal body structure, such as an organ.

### BACKGROUND

Surgeons regularly examine internal regions of a patient's anatomy, e.g., in preparation for, or during, surgery. For example, doctors may use a colonoscope to examine a patient's intestine, a bronchoscope to examine a patient's lungs, a laparoscope to examine a gas-filled abdomen (including a region between organs), etc., in each case to determine whether a follow up surgery is desirable, to deliver localized chemotherapy, localized excisions, delivery of treatments, etc. Such examinations may employ remote camera devices and other instruments to inspect the internal body structure of the patient in a minimally invasive manner. While such examinations already readily occur in non-robotic contexts, as robotic surgeries become more frequent, such examinations may be integrated into the surgeon's protocols before, during, or after the robotic surgery.

It may be difficult to quickly and efficiently assess the quality and character of these examinations. For example, surgeons wishing to review their progress across surgeries over time, administrators wishing to verify reimbursement billing codes, data scientists seeking to examine surgical data, etc., may each be obligated to sit through hours of raw, unprocessed video. Naturally, the reviewers' finite attention spans and the variety of disparate artifacts appearing in different surgical contexts may render such manual review inefficient and ineffective. The reviewers may fail to recognize events occurring in the surgery or may misinterpret such events, may not readily perceive the holistic content of the data, may not readily discern the adequacy of the review's inspection of an internal body structure, etc. Offline review of raw procedure video is time consuming, requiring that many irrelevant sequences be ignored, while real-time review of such videos may distract team members from performing their responsibilities during the surgery.

Accordingly, there exists a need for systems and methods readily facilitating review of a surgical procedure, which not only automate the more routine tasks of the review, but which readily highlight and call attention to data in a manner understandable to a wide audience of specialists. Ideally, such a presentation would be available both offline, following the surgery, and online, during a surgical operation, to facilitate real-time review of an examination. Such real-time review may be desirable, e.g., to help a human operator to bookmark interesting portions of the procedure, quickly verify the recognition of adverse growths, and to immediately recognize and compensate for lacunae in the operator's review (e.g., revisiting a portion of the internal body structure while the patient is still prepared for surgery is much more efficient than deciding to revisit following closure).

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation as may occur in relation to some embodiments;

FIG. 1B is a schematic view of various elements appearing in a surgical theater during a surgical operation employing a surgical robot as may occur in relation to some embodiments;

FIG. 2A is a schematic illustration of an organ, in this example a large intestine, with a cutaway view revealing the progress of a colonoscope during a surgical examination as may occur in connection with some embodiments;

FIG. 2B is a schematic illustration of an colonoscope distal tip as may be used in connection with some embodiments;

FIG. 2C is a schematic illustration of a portion of a colon with a cutaway view revealing a position of a colonoscope relative to a plurality of haustra;

FIG. 2D is a schematic representation of a camera-acquired visual image and a corresponding depth frame acquired from the perspective of the camera of the colonoscope depicted in FIG. 2C;

FIG. 2E is a pair of images depicting a grid-like pattern of orthogonal rows and columns in perspective, as captured from a colonoscope camera having a rectilinear view and a colonoscope camera having a fisheye view, each of which may be used in connection with some embodiments;

FIG. 3A is a schematic illustration of a computer-generated three-dimensional model of a large intestine with portions of the model highlighted, in a first perspective, as may occur in some embodiments;

FIG. 3B is a schematic illustration of the computer-generated three-dimensional model of FIG. 3A in a second perspective;

FIG. 3C is a schematic illustration of the computer-generated three-dimensional model of FIG. 3A in a third perspective;

FIGS. 4A-C are temporally successive schematic two-dimensional cross-section representations of a colonoscope progressing through a large intestine, as may occur in some embodiments;

FIGS. 4D-F are two dimensional schematic representations of depth frames generated from the corresponding fields of view depicted in FIGS. 4A-C, as may occur in some embodiments;

FIG. 4G is a schematic two-dimensional representation of a fusion operation between the depth frames of FIGS. 4D-F to create a consolidated representation, as may occur in some embodiments;

FIG. 5 is a flow diagram illustrating various operations in an example process for generating a computer model of at least a portion of an internal body structure, such as an organ, as may be implemented in some embodiments;

FIG. 6 is an example processing pipeline for generating at least a portion of a three-dimensional model of a large intestine from a colonoscope data capture, as may be implemented in some embodiments;

FIG. 7A is an example processing pipeline for determining a depth map and coarse local pose from colonoscope images using two distinct neural networks, as may be implemented in some embodiments;

FIG. 7B is an example processing pipeline for determining a depth map and coarse local pose from colonoscope images using a single neural network, as may be implemented in some embodiments;

FIG. 8A is a flow diagram illustrating various operations in a neural network training process as may be performed upon the networks of FIGS. 7A and 7B in some embodiments;

FIG. 8B is a bar plot depicting an exemplary set of training results for the process of FIG. 8A as may occur in connection with some embodiments;

FIG. 9A is a flow diagram illustrating various operations in a new fragment determination process as may be implemented in some embodiments;

FIG. 9B is a schematic side-view representation of an endoscope's successive fields of view as relates to a frustum overlap determination, as may occur in some embodiments;

FIG. 9C is a schematic temporal series of cross-sectional views depicting a colonoscope colliding with a sidewall of a colon and the resulting changes in the colonoscope camera's field of view, as may occur in connection with some embodiments;

FIG. 9D is a schematic representation of a collection of fragments corresponding to the collision of FIG. 9C, as may be generated in some embodiments;

FIG. 9E is a schematic network diagram illustrating various keyframe relations following graph network pose optimization operations, as may be occur in some embodiments;

FIG. 9F is a schematic diagram illustrating fragments with associated Truncated Signed Distance Function (TSDF) meshes relative to a full model TSDF mesh as may be generated in some embodiments;

FIG. 10A is an example pose processing pipeline as may be implemented in some embodiments;

FIG. 10B is an example pose processing pipeline as may be implemented in some embodiments;

FIG. 11A is a flow diagram illustrating various operations in an example hole identification process as may be implemented in some embodiments;

FIG. 11B is a schematic series of three-dimensional data structures as may be generated in accordance with the process of FIG. 11A;

FIG. 12A is a series of schematic representations of a three-dimensional computer-generated model for a portion of a large intestine, specifically depicting a complete model, an incomplete model, a planar reconstructed model, and a projected reconstructed model, as may occur in some embodiments;

FIG. 12B is a series of schematic representations of a three-dimensional computer-generated model for a portion of a large intestine, specifically a pair of haustra, depicting a complete model, an incomplete model, a planar reconstructed model, and a projected reconstructed model, as may occur in some embodiments;

FIG. 12C is a series of schematic representations of a three-dimensional computer-generated model of a hollow cylinder in cross-sectional view, depicting a complete model, an incomplete model, a planar reconstructed model, and a projected reconstructed model;

FIG. 13A is a schematic representation of a plurality of model states prepared for centerline determination and path assessment, as may be implemented in some embodiments;

FIG. 13B is a flow diagram illustrating various operations in an example process for determining and representing path quality to a reviewer, as may be implemented in some embodiments;

FIG. 13C is a schematic representation of voxel grid depth frame representations, as may be implemented in some embodiments;

FIG. 13D is a flow diagram illustrating various operations in an example process for preparing model data for supplementation by a machine learning system, as may be implemented in some embodiments;

FIG. 14 is schematic representation of example landmarks within a region of a body, here, a large intestine, as may be occur in some embodiments;

FIG. 15A is an example vision neural network processing pipeline for landmark detection, as may be implemented in some embodiments;

FIG. 15B is an example attention implementing neural network, specifically, a transformer encoder neural network, as may be used in conjunction with the vision neural network processing pipeline of FIG. 15A, as may be implemented in some embodiments;

FIG. 16 is an example pipeline for configuring and deploying a landmark recognition system, as may be implemented in some embodiments;

FIG. 17A is a schematic representation of a cropping operation as may be implemented in some embodiments;

FIG. 17B is a schematic representation of a histogram clipping operation as may be implemented in some embodiments;

FIG. 17C is a schematic representation of colonoscope configurations producing specular reflections, as may occur in some embodiments;

FIG. 17D is a schematic representation of an in-painting reflection removal operation as may be implemented in some embodiments;

FIG. 17E is a flow diagram illustrating various operations in an example process for performing a cropping operation as depicted in FIG. 17A, as may be implemented in some embodiments;

FIG. 17F is a flow diagram illustrating various operations in an example process for performing a histogram clipping operation as depicted in FIG. 17B, as may be implemented in some embodiments;

FIG. 17G is a flow diagram illustrating various operations in an example process for performing an in-painting reflection removal operation as depicted in FIG. 17D, as may be implemented in some embodiments;

FIG. 18 is a schematic representation of an example graphical user interface (GUI) for offline or real-time internal body structure data review, as may be implemented in some embodiments;

FIG. 19 is a flow diagram illustrating various operations in a process for presenting internal body structure review data, as may be implemented in some embodiments;

FIG. 20 is a flow diagram illustrating various operations in a process for scoring, or marking, internal body structure review data, e.g., for adjusting the model rendering, as may be implemented in some embodiments;

FIG. 21A is a schematic representation of inputs to a GUI data management system as may occur in some embodiments;

FIG. 21B is a flow diagram illustrating various operations in an example process for preparing incoming examination data for presentation, as may be implemented in some embodiments;

FIG. 22A is a schematic representation of an example mesh alignment operation as may be performed in some embodiments;

FIG. 22B a flow diagram illustrating various operations in an example process for performing a mesh alignment, as may be implemented in some embodiments;

FIG. 23A is a schematic representation of example elements in a GUI for quickly reviewing examination data collected in connection with an organ or other internal structure, as may be implemented in some embodiments, when a cursor is in a first position;

FIG. 23B is a schematic representation of the elements from FIG. 23A when the cursor is in a second position;

FIG. 23C is a schematic representation of the elements from FIG. 23A when the cursor is in a third position;

FIG. 24A is a schematic representation of example elements in a GUI for quickly reviewing temporally successive examination data collected in connection with an organ or other internal structure, as may be implemented in some embodiments, at a first position in the data playback;

FIG. 24B is a schematic representation of the elements of FIG. 24A at a second position in the data playback;

FIG. 24C is a schematic representation of the elements of FIG. 24A at a third position in the data playback;

FIG. 25A is a schematic representation of an element variation in the elements of FIG. 24A, as may be implemented in some embodiments;

FIG. 25B is a schematic representation of the element variation of FIG. 25A, wherein the model is rotated relative to its orientation in FIG. 25A;

FIG. 25C is a schematic representation of GUI elements from FIG. 24B wherein the model is in the orientation of FIG. 25B;

FIG. 26A is a flow diagram illustrating various operations in an example process for managing a GUI, such as a GUI of FIGS. 23A-C, 24A-C, and 25A-C, as may be implemented in some embodiments;

FIG. 26B is a flow diagram illustrating various operations in an example process for managing a GUI during playback, as may be implemented in some embodiments;

FIG. 27A is a flow diagram illustrating various operations in an example process for performing a supplemental frame-based landmark determination, as may be implemented in some embodiments;

FIG. 27B is a schematic presentation of a video image frame projection upon a portion of a model rendering as may be performed in some embodiments; and

FIG. 28 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

### Example Surgical Theaters Overview

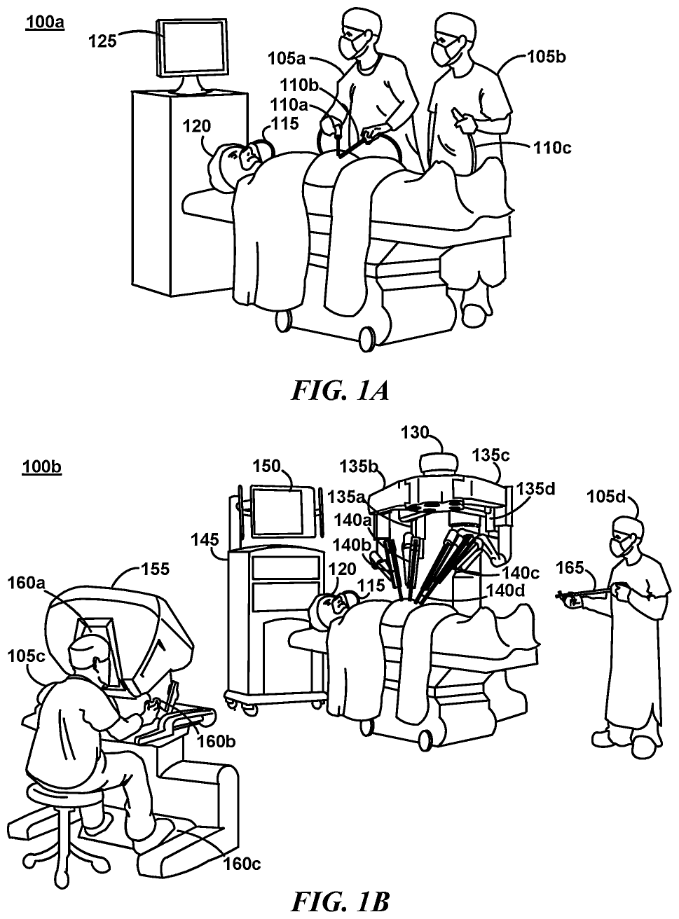

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image acquiring endoscope, etc. and a mechanical end effector 110a such as scissors, retractors, a dissector, etc.

The visualization tool 110b provides the surgeon 105a with an interior view of the patient 120, e.g., by displaying visualization output from a camera mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is an endoscope, one will appreciate that, in some embodiments, visualization tool 110b may capture depth data instead of, or in addition to, two-dimensional image data (e.g., with a laser rangefinder, stereoscopy, etc.). Accordingly, one will appreciate that it may be possible to apply various of the two-dimensional operations discussed herein, mutatis mutandis, to such three-dimensional depth data when such data is available.

A single surgery may include the performance of several groups of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a surgical robot, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc. the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement within display 160a, etc. may all be recorded. Similarly, patient side cart 130 may record tool activations (e.g., the application of radiative energy, closing of scissors, etc.), movement of end effectors, etc. throughout the surgery. In some embodiments, the data may have been recorded using an in-theater recording device, such as an Intuitive Data Recorder™ (IDR), which may capture and store sensor data locally or at a networked location.

### Example Organ Data Capture Overview

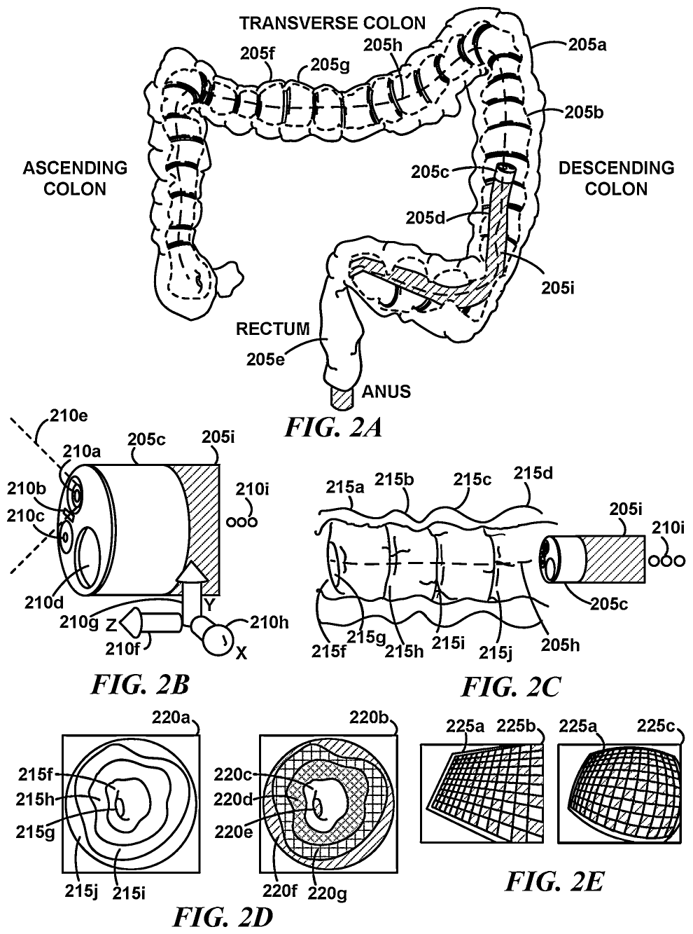

Whether in non-robotic surgical theater 100a or in robotic surgical theater 100b, there may be situations where surgeon 105a, assisting member 105b, the operator 105c, assisting member 105d, etc. seek to examine an organ or other internal body structure of the patient 120 (e.g., using visualization tool 110b or 140d). For example, as shown in FIG. 2A and revealed via cutaway 205b, a colonoscope 205d may be used to examine a large intestine 205a. While this detailed description will use the large intestine and colonoscope as concrete examples with which to facilitate the reader's comprehension, one will readily appreciate that the disclosed embodiments need not be limited to large intestines and colonoscopes, and indeed are here explicitly not contemplated as being so limited. Rather, one will appreciate that the disclosed embodiments may likewise be applied in conjunction with other organs and internal structures, such as lungs, hearts, stomachs, arteries, veins, urethras, regions between organs and tissues, etc. and with other instruments, such as laparoscopes, thorascopes, sensor-bearing catheters, bronchoscopes, ultrasound probes, miniature robots (e.g., swallowed sensor platforms), etc. Many such organs and internal structures will include folds, outcrops, and other structures, which may occlude portions of the organ or internal structure from one or more perspectives. For example, the large intestine 205a shown here includes a series of pouches known as haustra, including haustrum 205f and haustrum 205g. Thoroughly examining the large intestine despite occlusions in the field of view precipitated by these haustra and various other challenges, including possible limitations of the visualization tool itself, may be very difficult for the surgeon or automated system.

In the depicted example, the colonoscope 205d may navigate through the large intestine by adjusting bending section 205i as the operator, or automated system, slides colonoscope 205d forward. Bending section 205i may likewise be adjusted so as to orient a distal tip 205c in a desired orientation. As the colonoscope proceeds through the large intestine 205a, possibly all the way from the descending colon, to the transverse colon, and then to the ascending colon, actuators in the bending section 205i may be used to direct the distal tip 205c along a centerline 205h of the intestines. Centerline 205h is a path along points substantially equidistant from the interior surfaces of the large intestine along the large intestine's length. Prioritizing the motion of colonoscope 205d along centerline 205h may reduce the risk of colliding with an intestinal wall, which may harm or cause discomfort to the patient 120. While the colonoscope 205d is shown here entering via the rectum 205e, one will appreciate that laparoscopic incisions and other routes may also be used to access the large intestine, as well as other organs and internal body structures of patient 120.

FIG. 2B provides a closer view of the distal tip 205c of colonoscope 205d. This example tip 205c includes a visual image camera 210a (which may capture, e.g., color or grayscale images), light source 210c, irrigation outlet 210b, and instrument bay 210d (which may house, e.g., a cauterizing tool, scissors, forceps, etc.), though one will readily appreciate variations in the distal tip design. For clarity, and as indicated by the ellipsis 210i, one will appreciate that the bending section 205i may extend a considerable distance behind the distal tip 205c.

As previously mentioned, as colonoscope 205d advances and retreats through the intestine, joints, or other bendable actuators within bending section 205i, may facilitate movement of the distal tip 205c in a variety of directions. For example, with reference to the arrows 210f, 210g, 210h, the operator, or an automated system, may generally advance the colonoscope tip 205c in the Z direction represented by arrow 210f. Actuators in bendable portion 205i may allow the distal end 205c to rotate around the Y axis or X axis (perhaps simultaneously), represented by arrows 210g and 210h respectively (thus analogous to yaw and pitch, respectively). In this manner, camera 210a's field of view 210e may be adjusted to facilitate examination of structures other than those appearing directly before the colonoscope's direction of motion, such as regions obscured by the haustral folds.

Specifically, FIG. 2C is a schematic illustration of a portion of a large intestine with a cutaway view revealing a position of the colonoscope tip 205c relative to a plurality of haustral annular ridges. Between each of haustra 215a, 215b, 215c, 215d may lie an interstitial tissue forming an annular ridge. In this example, annular ridge 215h is formed between haustra 215a, 215b, annular ridge 215i is formed between haustra 215b, 215c, and annular ridge 215j is formed between haustra 215c, 215d. While the operator may wish the colonoscope to generally travel a path down the centerline 205h of the colon, so as to minimize discomfort to the patient, the operator may also wish for bendable portion 205i to reorient the distal tip 205c such that the camera 210a's field of view 210e may observe portions of the colon occluded by the annular ridges.

Regions further from the light source 210c may appear darker to camera 210a than regions closer to the light source 210c. Thus, the annular ridge 215j may appear more luminous in the camera's field of view than opposing wall 215f, and aperture 215g may appear very, or entirely, dark to the camera 210a. In some embodiments, the distal tip 205c may include a depth sensor, e.g., in instrument bay 210d. Such a sensor may determine depth using, e.g., time-of-flight photon reflectance data, sonography, a stereoscopic pair of visual image cameras (e.g., on extra camera in addition to camera 210a) etc. However, various embodiments disclosed herein contemplate estimating depth data based upon the visual images of the single visual image camera 210a upon the distal tip 205c. For example, a neural network may be trained to recognize distance values corresponding to images from the camera 210a (e.g., as variations in surface structures and the luminosity resulting from reflected light of light 210c at varying distance may provide sufficient correlations with depth between successive images for a machine learning system to make a depth prediction). Some embodiments may employ a six degree of freedom guidance sensor (e.g., the 3D Guidance® sensors provided by Northern Digital Inc.) in lieu of the pose estimation methods described herein, or in combination with those methods, such that the methods described herein and the six degree of freedom sensors provide complementary confirmation of one another's results.

Thus, for clarity, FIG. 2D depicts a visual image and a corresponding schematic representation of a depth frame acquired from the perspective of the camera of colonoscope depicted in FIG. 2C. Here, annular ridge 215j occludes a portion of annular ridge 215i, which itself occludes a portion of annular ridge 215h, while annular ridge 215h occludes a portion of the wall 215f. While the aperture 215g is within the camera's field of view, the aperture is sufficiently distant from the light source that it may appear entirely dark.

With the aid of a depth sensor, or via image processing of image 220a (and possibly a preceding or succeeding image following the colonoscope's movement) using systems and methods discussed herein, etc., a corresponding depth frame 220b may be generated, which corresponds to the same field of view producing visual image 220a. As shown in this example, the depth frame 220b assigns a depth value to some or all of the pixel locations in image 220a (though one will appreciate that the visual image and depth frame will not always have values directly mapping pixels to depth values, e.g., where the depth frame is of smaller dimensions than the visual image). One will appreciate that the depth frame, comprising a range of depth values, may itself be presented as a grayscale image in some embodiments (e.g., the largest depth value mapped to value of 0, the shortest depth value mapped to 255, and the resulting mapped values presented as a grayscale image). Thus, the annular ridge 215j may be associated with a closest set of depth values 220f, the annular ridge 215i may be associated with a further set of depth values 220g, the annular ridge 215h may be associated with a yet further set of depth values 220d, the back wall 215f may be associated with a distant set of depth values 220c, and the aperture 215g may be beyond the depth sensing range (or entirely black, beyond the light source's range) leading to the largest depth values 220e (e.g., a value corresponding to infinite, or unknown, depth). While a single pattern is shown for each annular ridge in this schematic figure to facilitate comprehension by the reader, one will appreciate that the annular ridges will rarely present a flat surface in the X-Y plane (per arrows 210h and 210g) of the distal tip. Consequently many of depth values within, e.g., set 220f, are unlikely to be the exact same value.

While visual image camera 210a may capture rectilinear images one will appreciate that lenses, post-processing, etc. may be applied in some embodiments such that images captured from camera 210a are other than rectilinear. For example, FIG. 2E is a pair of images 225b, 225c depicting a grid-like checkered pattern 225a of orthogonal rows and columns in perspective, as captured from a colonoscope camera having a rectilinear view and a colonoscope camera having a fisheye view, respectively. Such a checkered pattern may facilitate determination of a given camera's intrinsic parameters. One will appreciate that the rectilinear view may be achieved by undistorting the fisheye view, once the intrinsic parameters of the camera are known (which may be useful, e.g., to normalize disparate sensor systems to a similar form recognized by a machine learning architecture). A fisheye view may allow the user to readily perceive a wider field of view than in the case of the rectilinear perspective. As the focal point of the fisheye lens, and other details of the colonoscope, such as the light 210c luminosity, may vary between devices and even across the same device over time, it may be necessary to recalibrate various processing methods for the particular device at issue (consider the device's “intrinsics”, e.g., such as focal-length, principal points, distortion coefficients etc.) or to at least anticipate device variation when training and configuring a system.

### Example Computer Generated Organ Model

During or following an examination of an internal body structure (such as large intestine 205a) with a camera system (e.g., camera 210a), it may be desirable to generate a corresponding three-dimensional model of the organ or examined cavity. For example, various of the disclosed embodiments may generate a Truncated Signed Distance Function (TSDF) volume model, such as the TSDF model 305 of the large intestine 205a, based upon the depth data captured during the examination (while TSDF is offered here as an example to facilitate the reader's comprehension, one will appreciate that any three-dimensional mesh data format may suffice). The model may be textured with images captured via camera 210a or may, e.g., be colored with a vertex shader. For example, where the colonoscope traveled inside the large intestine, the model may include an inner and outer surface, the inner rendered with the textures captured during the examination and the outer surface shaded with vertex colorings. In some embodiments, only the inner surface may be rendered, or only a portion of the outer surface may be rendered, so that the reviewer may readily examine the organ interior.

Such a computer-generated model may be useful for a variety of purposes. For example, portions of the model may be differently textured, highlighted via an outline (e.g., the region's contour from the perspective of the viewer being projected upon the texture of a billboard vertex mesh surface in front of the model), called out with three dimensional markers, or otherwise identified, which are associated with, e.g.: portions of the examination bookmarked by the operator, portions of the organ found to have received inadequate review as determined by various embodiments disclosed herein, organ structures of interest (such as polyps, tumors, abscesses, etc.), etc. For example, portions 310a and 310b of the model may be vertex shaded, or outlined, in a color different or otherwise distinct from the rest of the model 305, to call attention to inadequate review by the operator, e.g., where the operator failed to acquire a complete image capture of the organ region, moved too quickly through the region, acquired only a blurred image of the region, viewed the region while it was obscured by smoke, etc. Though a complete model of the organ is shown in this example, one will appreciate that an incomplete model may likewise be generated, e.g., in real-time during the examination, following an incomplete examination, etc. In some embodiments, the model may be a non-rigid 3D reconstruction (e.g., incorporating a physics model to represent the behavior of tissues with varying stiffness).

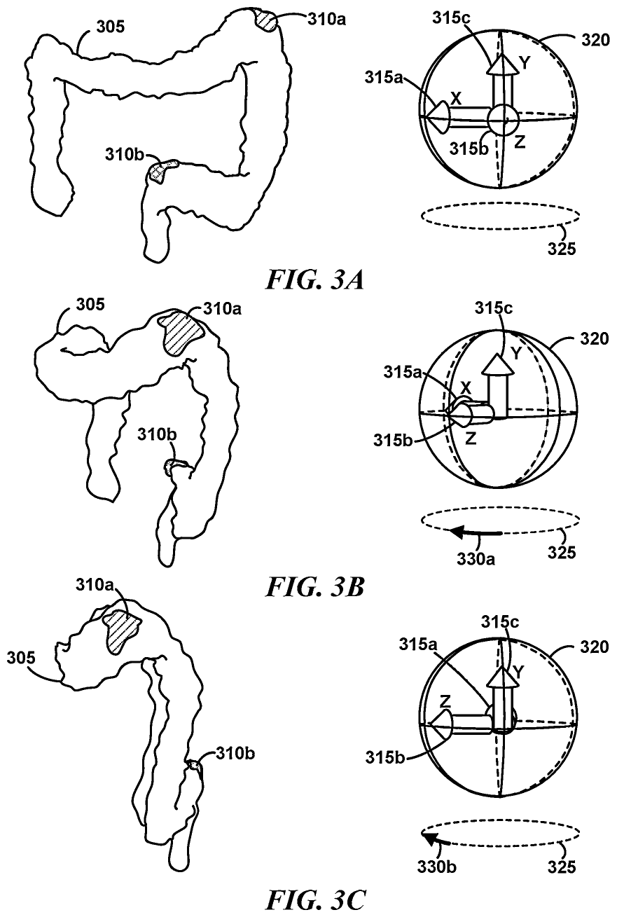

For clarity, each of FIGS. 3A, 3B, 3C depict the three-dimensional model 305 from a different perspective. Specifically, a coordinate reference 320, having X-Y-Z axes represented by arrows 315a, 315c, 315b respectively, is provided for the reader's reference. If the model were rendered about coordinate reference 320 at the model's center, then FIG. 3B shows the model 305 rotated approximately 40 degrees 330a around the Y-axis, i.e., in the X-Z plane 325, relative to the model 305's orientation in FIG. 3A. Similarly, FIG. 3C depicts the model 305 further rotated approximately an additional 40 degrees 330b to an orientation at nearly a right angle to that of the orientation in FIG. 3A. One will appreciate that the model 305 may be rendered only from the interior of the organ (e.g., where the colonoscope appeared), only the exterior, or both the interior or exterior (e.g., using two, complementary texture meshes). Where the only data available is for the interior of the organ, the exterior texture may be vertex shaded, textured with a synthetic texture approximating that of the actual organ, simply transparent, etc. In some embodiments, only the exterior is rendered with vertex shading. As discussed herein, a reviewer may be able to rotate the model in a manner analogous to FIGS. 3A, 3B, 3C, as well as translate, zoom, etc. so as, e.g., to more closely investigate identified regions 310a, 310b, to plan follow-up surgeries, to assess the organ's relation to a contemplated implant (e.g., a surgical mesh, fiducial marker, etc.), etc.

### Example Frame Generation and Consolidation Operations

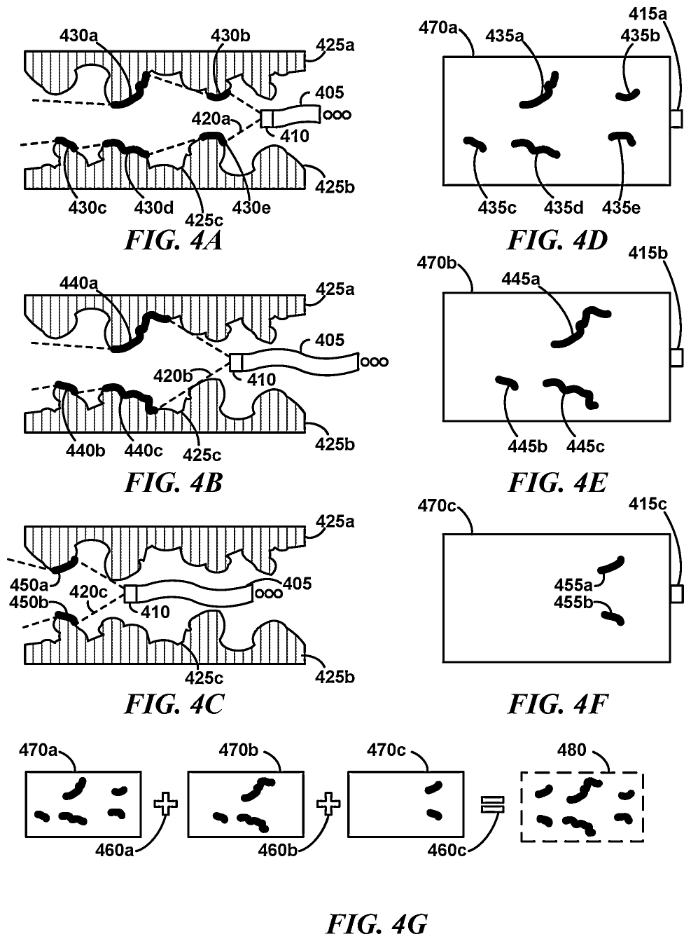

As depth data may be incrementally acquired throughout the examination, the data may be consolidated to facilitate creation of a corresponding three-dimensional model (such as model 305) of all or a portion of the internal body structure. For example, FIGS. 4A-C present temporally successive schematic two-dimensional cross-sectional representations of a colonoscope field of view, corresponding to the actual three-dimensional field of view, as the colonoscope proceeds through a colon.

Specifically, FIG. 4A depicts a two-dimensional cross sectional view of the interior of a colon, represented by top portion 425a and bottom portion 425b. As discussed, the colon interior, like many body interiors, may contain various irregular surfaces, e.g., where haustra are joined, where polyps form, etc. Accordingly, when the colonoscope 405 is in the position of FIG. 4A the camera coupled with distal tip 410 may have an initial field of view 420a. As the irregular surface may occlude portions of the colon interior, only certain surfaces, specifically the surfaces 430a, 430b, 430c, 430d, and 430e may be visible to the camera (and/or depth sensor) from this position. Again, as this is a cross sectional view similar to FIG. 2C, one will appreciate that such surfaces may correspond to the annular ridge surfaces appearing in the image 220a. That is, while surfaces are represented here by lines, one will appreciate that these surfaces may correspond to three dimensional structures, e.g., to the annular ridges between haustra, such as the annular ridges 215h, 215i, 215j. As a result of the limited field of view, a surgeon may have not yet viewed an occluded region, such as the region 425c outside the field of view 420a. One will appreciate that such limitations upon the field of view may be present whether the camera image is rectilinear, fisheye, etc.

As the colonoscope 405 advances further into the colon (from right to left in this depiction) as shown in FIG. 4B the camera's field of view 420b may now perceive surfaces 440a, 440b, and 440c. Naturally, portions of these surfaces may coincide with previously viewed portions of surfaces, as in the case of surfaces 430a and 440a. If the colonoscope's field of view continues to advance linearly, without adjustment (e.g., rotation of the distal tip via the bendable section 205i), portions of the occluded surface may remain unviewed. Here, e.g., the region 425c has still not appeared within the camera's field of view 420b despite the colonoscope's advancement. Similarly, as the colonoscope 405 advances to the position of FIG. 4C, surfaces 450a and 450b may now be visible in field of view 420c, but, unfortunately, the colonoscope will continue to have passed the region 425c without the region 425c appearing in the field of view.

One will appreciate that throughout colonoscope 405's progress, depth values corresponding to the interior structures before the colonoscope may be generated either in real-time during the examination or by post-processing of captured data after the examination. For example, where the distal tip 205c does not include a sensor specifically designed for depth data acquisition, the system may instead use the images from the camera to infer depth values (an operation which may occur in real-time or near real-time using the methods described herein). Various methods exist for determining depth values from images including, e.g., using a neural network trained to convert visual image data to depth values. For example, one will appreciate that self-supervised approaches for producing a network inferring depth from monocular images may be used, such as that found in the paper “Digging Into Self-Supervised Monocular Depth Estimation” appearing as arXiv preprint arXiv: 1806.01260v4 and by Clément Godard, Oisin Mac Aodha, Michael Firman, and Gabriel Brostow, and as implemented in the Monodepth2 self-supervised model described in that paper. However, such methods do not specifically anticipate the unique challenges present in this endoscopic context and may be modified as described herein. Where the distal tip 205c does include a depth sensor, or where stereoscopic visual images are available, the depth values from the various sources may be corroborated by the values from the monocular image approach.

Thus, a plurality of depth values may be generated for each position of the colonoscope at which data was captured to produce a corresponding depth data “frame.” Here, the data in FIG. 4A may produce the depth frame 470a of FIG. 4D, the data in FIG. 4B may produce the depth frame 470b of FIG. 4E, and the data in FIG. 4C may produce the depth frame 470c of FIG. 4F. Thus, depth values 435a, 435b, 435c, 435d, and 435e, may correspond to surfaces 430a, 430b, 430c, 430d, and 430e respectively. Similarly, depth values 445a, 445b, and 445c may correspond to surfaces 440a, 440b, and 440c, respectively, and depth values 455a and 455b may correspond to surfaces 450a and 450b.

Note that each depth frame 470a, 470b, 470c is acquired from the perspective of the distal tip 410, which may serve as the origin 415a, 415b, 415c for the geometry of each respective frame. Thus, each of the frames 470a, 470b, 470c must be considered relative to the pose of the distal tip at the time of data capture and globally reoriented if the depth data in the resulting frames is to be consolidated, e.g., to form a three-dimensional representation of the organ as a whole (such as model 305). This process, known as stitching or fusion, is shown schematically in FIG. 4G wherein the depth frames 470a, 470b, 470c are combined 460a, 460b to form 460c a consolidated frame 480. One will appreciate a variety of methods for stitching together frames in this manner, including example methods described herein.

### Example Data Processing Operations

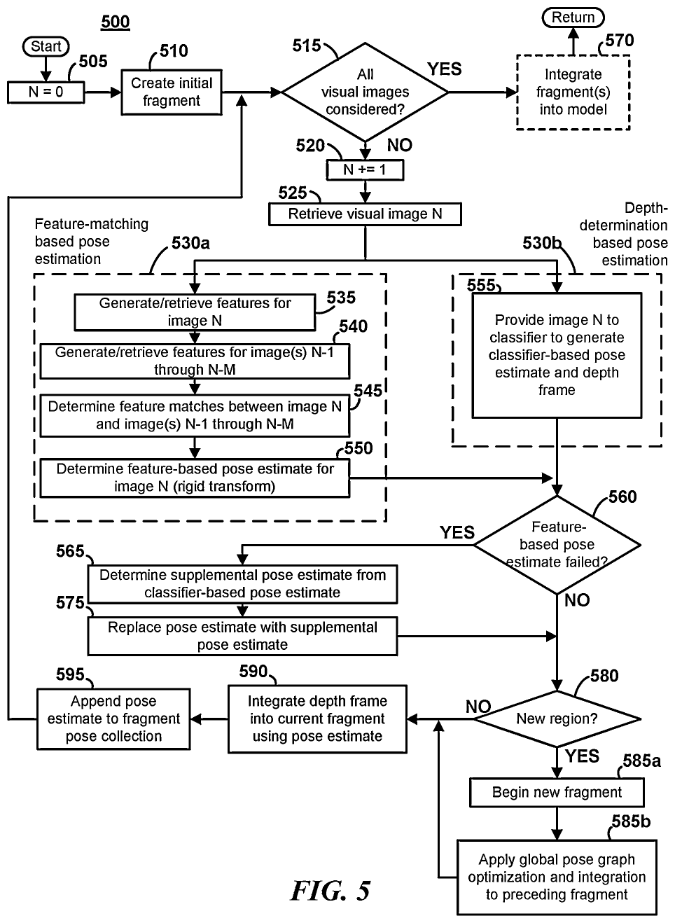

FIG. 5 is a flow diagram illustrating various operations in an example process 500 for generating a computer model of at least a portion of an internal body structure, as may be implemented in some embodiments. At block 505, the system may initialize a counter N to 0 (one will appreciate that the flow diagram is merely exemplary and selected to facilitate the reader's understanding, consequently, many embodiments may not employ such a counter or the specific operations disclosed in FIG. 5). At block 510 the computer system may allocate storage for an initial fragment data structure. As explained in greater detail herein, a fragment is a data structure comprising one or more depth frames, facilitating creation of all or a portion of a model. In some embodiments, the fragment may contain data relevant to a sequence of consecutive frames depicting a similar region of the internal body structure and may share a large intersection area over that region. Thus, a fragment data structure may include memory allocated to receive RGB visual images, visual feature correspondences between visual images, depth frames, relative poses between the frames within the fragment, timestamps, etc. At blocks 515 and 520 the system may then iterate over each image in the captured video, incrementing the counter accordingly, and then retrieving the corresponding next successive visual image of the video at block 525.

As shown in this example, the visual image retrieved at block 525 may then be processed by two distinct subprocesses, a feature-matching based pose estimation subprocess 530a and a depth-determination based pose estimation subprocess 530b, in parallel. Naturally, however, one will appreciate that the subprocesses may instead be performed sequentially. Similarly, one will appreciate that parallel processing need not imply two distinct processing systems, as a single system may be used for parallel processing with, e.g., two distinct threads (as when the same processing resources are shared between two threads), etc.

Feature-matching based pose estimation subprocess 530a determines a local pose from an image using correspondences between the image's features (such as Scale-Invariant Feature Transforms (SIFT) features) and such features as they appear in previous images. For example, one may use the approach specified in the paper “BundleFusion: Real-time Globally Consistent 3D Reconstruction” appearing as arXiv preprint arXiv: 1604.01093v3 and by Angela Dai, Matthias Niessner, Michael Zollhofer, Shahram Izadi, and Christian Theobalt, specifically, the feature correspondence for global Pose Alignment described in section 4.1 of that paper, wherein the Kabsch algorithm is used for alignment, though one will appreciate that the exact methodology specified therein need not be used in every embodiment disclosed here (e.g., one will appreciate that a variety of alternative correspondence algorithms suitable for feature comparisons may be used). Rather, at block 535, any image features may be generated from the visual image which are suitable for pose recognition relative to the previously considered images' features. To this end, one may use SIFT features (as in the “BundleFusion” paper referenced above), Speeded-Up Robust Features (SURF), Features from Accelerated Segment Test (FAST), Binary Robust Independent Elementary Features (BRIEF) descriptors as used, e.g., in Orientated FAST and Rotated BRIEF (ORB), Binary Robust Invariant Scalable Keypoints (BRISK), etc. In some embodiments, rather than use these conventional features, features may be generated using a neural network (e.g., from values in a layer of a UNet network, using the approach specified in the 2021 paper “LoFTR: Detector-Free Local Feature Matching with Transformers” available as arXiv preprint arXiv: 2104.00680v1 and by Jiaming Sun, Zehong Shen, Yuang Wang, Hujun Bao, and Xiaowei Zhou, using the approach specified in “SuperGlue: Learning Feature Matching with Graph Neural Networks”, available as arXiv preprint arXiv: 1911.11763v2 and by Paul-Edouard Sarlin, Daniel DeTone, Tomasz Malisiewicz, and Andrew Rabinovich, etc.). Such customized features may be useful when applied to a specific internal body context, specific camera type, etc.

The same type of features may be generated (or retrieved if previously generated) for previously considered images at block 540. For example, if M is 1, then only the previous image will be considered. In some embodiments, every previous image may be considered (e.g., M is N-1) similar to the “BundleFusion” approach of Dai, et al. The features generated at block 540 may then be matched with those features generated at block 535. These matching correspondences determined at block 545 may themselves then be used to determine a pose estimate at block 550 for the N^{th} image, e.g., by finding an optimal set of rigid camera transforms best aligning the features of the N through N-M images.

In contrast to feature-matching based pose estimation subprocess 530a, the depth-determination based pose estimation process 530b employs one or more machine learning architectures to determine a pose and a depth estimation. For example, in some embodiments, estimation process 530b considers the image N and the image N−1, submitting the combination to a machine learning architecture trained to determine both a pose and depth frame for the image, as indicated at block 555 (though not shown here for clarity, one will appreciate that where there are not yet any preceding images, or when N=1, the system may simply wait until a new image arrives for consideration; thus block 505 may instead initialize N to M so that an adequate number of preceding images exist for the analysis). One will appreciate that a number of machine learning architectures which may be trained to generate both a pose and depth frame estimate for a given visual image in this manner. For example, some machine learning architectures, similar to subprocess 530a, may determine the depth and pose by considering as input not only the N^{th} image frame, but by considering a number of preceding image frames (e.g., the N^{th} and N−1th images, the Nth through N-M images, etc.). However, one will appreciate that machine learning architectures which consider only the N^{th} image to produce depth and pose estimations also exist and may also be used. For example, block 555 may apply a single image machine learning architecture produced in accordance with various of the methods described in the paper “Digging Into Self-Supervised Monocular Depth Estimation” referenced above. The Monodepth2 self-supervised model described in that paper may be trained upon images depicting the endoscopic environment. Where sufficient real-world endoscopic data is unavailable for this purpose, synthetic data may be used. Indeed, while Godard et al.'s self-supervised approach with real world data does not contemplate using exact pose and depth data to train the machine learning architecture, synthetic data generation may readily facilitate generation of such parameters (e.g., as one can advance the virtual camera through a computer generated model of an organ in known distance increments) and may thus facilitate a fully supervised training approach rather than the self-supervised approach of their paper (though synthetic images may still be used in the self-supervised approach, as when the training data includes both synthetic and real-world data). Such supervised training may be useful, e.g., to account for unique variations between certain endoscopes, operating environments, etc., which may not be adequately represented in the self-supervised approach. Whether trained via self-supervised, fully supervised, or prepared via other training methods, the model of block 555 here predicts both a depth frame and pose for a visual image. One will appreciate a variety of methods for supplementing unbalanced synthetic and real-world datasets, including, e.g., the approach described in the 2018 paper “T2Net: Synthetic-to-Realistic Translation for Solving Single-Image Depth Estimation Tasks” available as arXiv preprint arXiv: 1808.01454v1 and by Chuanxia Zheng, Tat-Jen Cham, and Jianfei Cai, the approach described in the 2019 paper “Geometry-Aware Symmetric Domain Adaptation for Monocular Depth Estimation” available as arXiv preprint arXiv: 1904.01870v1 and by Shanshan Zhao, Huan Fu, Mingming Gong, and Dacheng Tao, the approach described in the paper “Unpaired Image-to-Image Translation using Cycle-Consistent Adversarial Networks” available as arXiv preprint arXiv: 1703.10593v7 and by Jun-Yan Zhu, Taesung Park, Phillip Isola, and Alexei A. Efros, and any suitable neural style transfer approach, such as that described in the paper “Deep Photo Style Transfer” available as arXiv preprint arXiv: 1703.07511v3 and by Fujun Luan, Sylvain Paris, Eli Shechtman, and Kavita Bala (e.g., suitable for results suggestive of photorealistic images).

Thus, as processing continues to block 560, the system may have available the pose determined at block 550, a second pose determined at block 555, as well as the depth frame determined at block 555. The pose determined at block 555 may not be the same as the pose determined at block 550, given their different approaches. If block 550 succeeded in finding a pose (e.g., a sufficiently large number of feature matches), then the process may proceed with the pose of block 550 and the depth frame generated at block 555 in the subsequent processing (e.g., transitioning to block 580).

However, in some situations, the pose determination at block 550 may fail. For example, where features failed to match at block 545, the system may be unable to determine a pose at block 550. While such failures may happen in the normal course of image acquisition, given the great diversity of body interiors and conditions, such failures may also result, e.g., when the operator moved the camera too quickly, resulting in a blurring of the N^{th} frame, making it difficult or impossible for features to be generated at block 535. Instrument occlusions, biomass occlusions, smoke (e.g., from a cauterizing device), or other irregularities may likewise result in either poor feature generation or poor feature matching. Naturally, if such an image is subsequently considered at block 545 it may again result in a failed pose recognition. In such situations, at block 560 the system may transition to block 565, preparing the pose determined at block 555 to serve in the place of the pose determined at block 550 (e.g., adjusting for differences in scale, format, etc., though substitution at block 575 without preparation may suffice in some embodiments) and making the substitution at block 575. In some embodiments, during the first iteration from block 515, as no previous frames exist with which to perform a match in the process 530a at block 540, the system may likewise rely on the pose of block 555 for the first iteration.

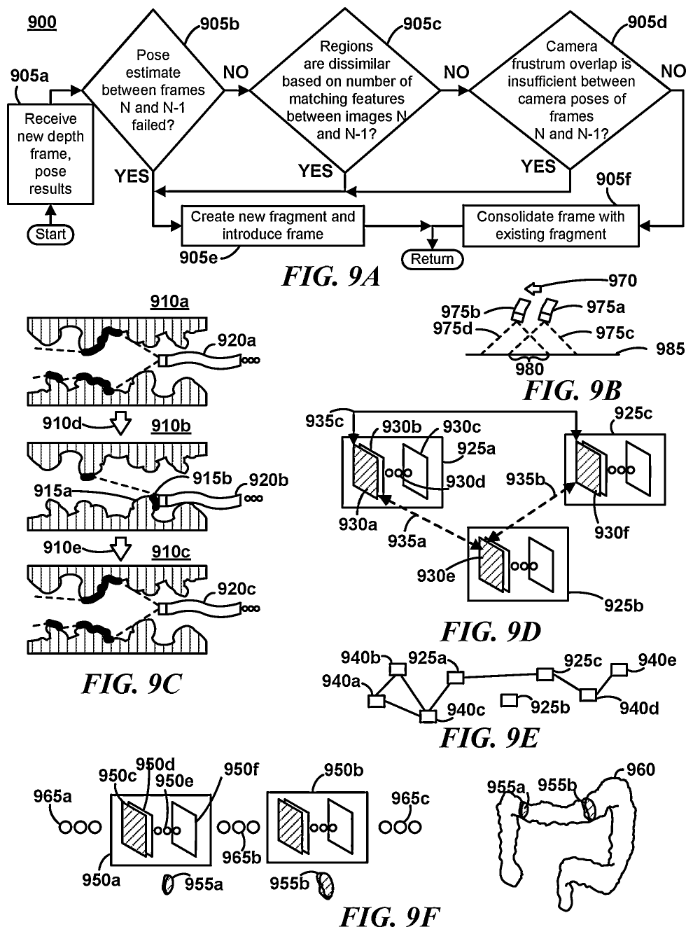

At block 580, the system may determine if the pose (whether from block 550 or from block 555) and depth frame correspond to the existing fragment being generated, or if they should be associated with a new fragment. One will appreciate a variety of methods for determining when a new fragment is to be generated. In some embodiments, new fragments may simply be generated after a fixed number (e.g., 20) of frames have been considered. In other embodiments, the number of matching features at block 545 may be used as a proxy for region similarity. Where a frame matches many of the features in its immediately prior frame, it may be reasonable to assign the corresponding depth frames to the same fragment (e.g., transition to block 590). In contrast, where the matches are sufficiently few, one may infer that the endoscope has moved to a substantially different region and so the system should begin a new fragment at block 585a. In addition, the system may also perform global pose network optimization and integration of the previously considered fragment, as described herein, at block 585b (for clarity, one will recognize that the “local” poses, also referred to as “coarse” poses, of blocks 550 and 555 are relative to successive frames, whereas the “global” pose is relative to the coordinates of the model as a whole). One example method for performing block 580 is provided herein with respect to the process 900 of FIG. 9A.

With the depth frame and pose available, as well as their corresponding fragment determined, at block 590 the system may integrate the depth frame with the current fragment using the pose estimate. For example, simultaneous localization and mapping (SLAM) may be used to determine the depth frame's pose relative to other frames in the fragment. As organs are often non-rigid, non-rigid methods such as that described in the paper “As-rigid-as-possible surface modeling” by Olga Sorkine and Marc Alexa, appearing in Symposium on Geometry processing. Vol. 4. 2007, may be used. Again, one will appreciate that the exact methodology specified therein need not be used in every embodiment. Similarly, some embodiments may employ methods from the DynamicFusion approach specified in the paper “DynamicFusion: Reconstruction and tracking of non-rigid scenes in real-time” by Richard A. Newcombe, Dieter Fox, and Steven M. Seitz, appearing in Proceedings of the IEEE conference on computer vision and pattern recognition. 2015. DynamicFusion may be appropriate as many of the papers referenced herein do not anticipate the non-rigidity of body tissue, nor the artifacts resulting from respiration, patient motion, surgical instrument motion, etc. The canonical model referenced in that paper would thus correspond to the keyframe depth frame described herein. In addition to integrating the depth frame with its peer frames in the fragment, at block 595, the system may append the pose estimate to a collection of poses associated with the frames of the fragment for future consideration (e.g., the collective poses may be used to improve global alignment with other fragments, as discussed with respect to block 570).

Once all the desired images from the video have been processed at block 515, the system may transition to block 570 and begin generating the complete, or intermediate, model of the organ by merging the one or more newly generated fragments with the aid of optimized pose trajectories determined at block 595. In some embodiments, block 570 may be foregone, as global pose alignment at block 585b may have already included model generation operations. However, as described in greater detail herein, in some embodiments not all fragments may be integrated into the final mesh as they are acquired, and so block 570 may include a selection of fragments from a network (e.g., a network like that described herein with respect to FIG. 9E).

### Example End-to-End Data Processing Pipeline

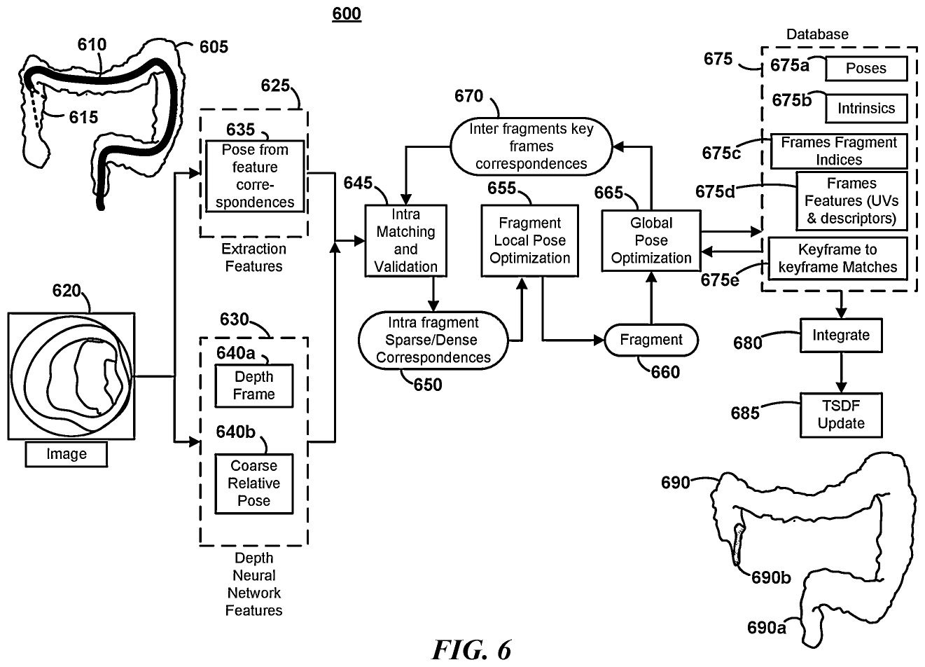

For additional clarity, FIG. 6 is a processing pipeline 600 for generating at least a portion of a three-dimensional model of a large intestine from a colonoscope data capture, as may be implemented in some embodiments. Again, while a large intestine is shown here to facilitate understanding, one will appreciate that the embodiments contemplate other organs and interior structures of patient 120.

Here, as a colonoscope 610 progresses through an actual large intestine 605, the camera or depth sensor may bring new regions of intestine 605 into view. At the moment depicted in FIG. 6, the region 615 of the intestine 605 is within view of the endoscope camera resulting in a two-dimensional visual image 620 of the region 615. The computer system may use the image 620 to generate both extraction features 625 (corresponding to process 530a) and depth neural network features 630 (corresponding to process 530b). In this example, the extraction features 625 produce the pose 635. Conversely, the depth neural network features 630 may include a depth frame 640a and pose 640b (though a neural network generating pose 640b may be unnecessary in embodiments where the pose 635 is always used).

As discussed, the computer system may use pose 635 and depth frame 640a in matching and validation operations 645, wherein the suitability of the depth frame and pose are considered. At blocks 650 and 655, the new frame may be integrated with the other frames of the fragment by determining correspondences therebetween and performing a local pose optimization. When the fragment 660 is completed, the system may align the fragment with previously collected fragments via global pose optimization 665 (corresponding, e.g., to block 585b). The computer system may then perform global pose optimization 665 upon the fragment 660 to orient the fragment 660 relative to the existing model. After creation of the first fragment, the computer system may also use this global pose to determine keyframe correspondences between fragments 670 (e.g., to generate a network like that described herein with respect to FIG. 9E).

Performance of the global pose optimization 665 may involve referencing and updating a database 675. The database may contain a record of prior poses 675a, camera calibration intrinsics 675b, a record of frame fragment indices 675c, frame features including corresponding UV texture map data (such as the camera images acquired of the organ) 675d, and a record of keyframe to keyframe matches 675e (e.g., like the network of FIG. 9E). The computer system may integrate 680 the database data (e.g., corresponding to block 570) at the conclusion of the examination, or in real-time during the examination, to update 685 or produce a computer generated model of the organ, such as a TSDF representation 690. In this example, the system is operating in real-time and is updating the preexisting portion of the TSDF model 690a with a new collection of vertices and textures 690b corresponding to the new fragment 660 generated for the region 615.

### Example End-to-End Data Processing Pipeline—Example Pose and Depth Pipeline

One will appreciate a number of methods for determining the coarse relative pose 640b and depth map 640a (e.g., at block 555). Naturally, where the examination device includes a depth sensor, the depth map 640a may be generated directly from the sensor (naturally, this may not produce a pose 640b). However, many depth sensors impose limitations, such as time of flight limitations, which may mitigate the sensor's suitability for in-organ data capture. Thus, it may be desirable to infer pose and depth data from visual images, as most examination tools will already be generating this visual data for the surgeon's review in any event.

Inferring pose and depth from an visual image can be difficult, particularly where only monocular, rather than stereoscopic, image data is available. Similarly, it can be difficult to acquire enough of such data, with corresponding depth values (if needed for training), to suitably train a machine learning architecture, such as a neural network. Some techniques do exist for acquiring pose and depth data from monocular images, such as the approach described in the “Digging Into Self-Supervised Monocular Depth Estimation” paper referenced herein, but these approaches are not directly adapted to the context of the body interior (Godard et al.'s work was directed to the field of autonomous driving) and so do not address various of this data's unique challenges.

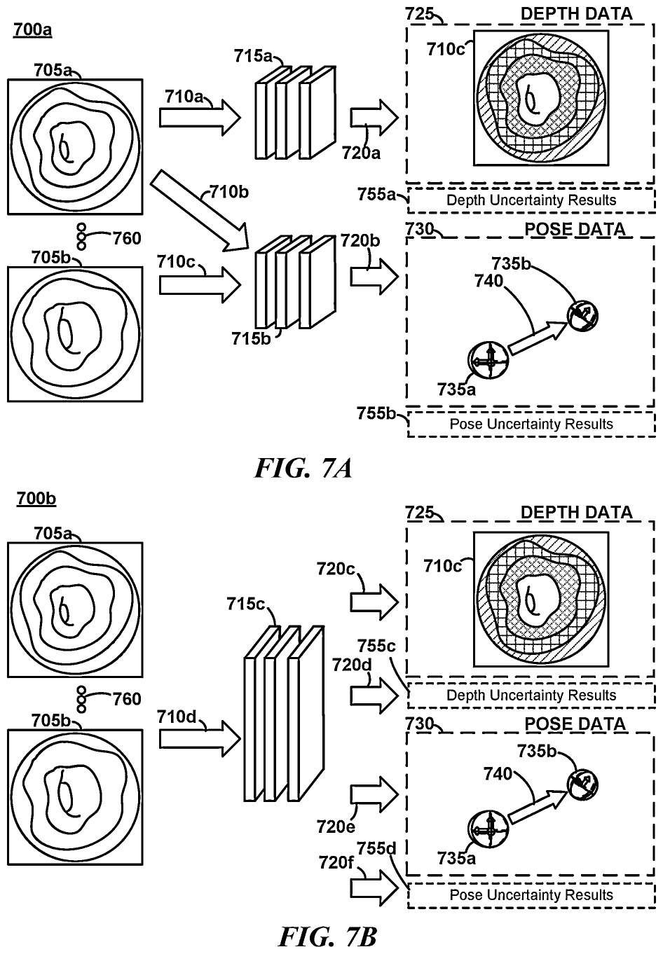

FIG. 7A depicts an example processing pipeline 700a for acquiring depth and pose data from monocular images in the body interior context. Here, the computer system considers two temporally successive image frames from an endoscope camera, initial image capture 705a and subsequent capture 705b after the endoscope has advanced forward through the intestine (though, as indicated by ellipsis 760, one will readily appreciate variations where more than two successive images are employed and the inputs to the neural networks may be adjusted accordingly). In the pipeline 700a, a computer system supplies 710a initial image capture 705a to a first depth neural network 715a configured to produce 720a a depth frame representation 725 (corresponding to depth data 640a). One will appreciate that where more than two images are considered, image capture 705a may be, e.g., the first of the images in temporal sequence. Similarly, the computer system supplies 710b, 710c both image 705a and image 705b to a second pose neural network 715b to produce 720b a coarse pose estimate 730 (corresponding to coarse relative pose 640b). Specifically, network 715b may predict a transform 740 explaining the difference in view between both image 705a (taken from orientation 735a) and image 705b (taken from orientation 735b). One will appreciate that in embodiments where more than two successive images are considered, the transform 740 may be between the first and last of the images, temporally. Where more than two input images are considered, all of the input images may be provided to network 715b.

Thus, in some embodiments, depth network 715a may be a UNet-like network (e.g., a network with substantially the same layers as UNet) configured to receive a single image input. For example, one may use the DispNet network described in the paper “Unsupervised Monocular Depth Estimation with Left-Right Consistency” available as an arXiv preprint arXiv: 1609.03677v3 and by Clément Godard, Oisin Mac Aodha, and Gabriel J. Brostow for the depth determination network 715a. As mentioned, one may also use the approach from “Digging into self-supervised monocular depth estimation” described above for the depth determination network 715a. Thus, the depth determination network 715a may be, e.g., a UNet with a ResNet (50) or ResNet (101) backbone and a DispNet decoder. Some embodiments may also employ depth consistency loss and masks between two frames during training as in the paper “Unsupervised scale-consistent depth and ego-motion learning from monocular video” available as arXiv preprint arXiv: 1908.10553v2 and by Jia-Wang Bian, Zhichao Li, Naiyan Wang, Huangying Zhan, Chunhua Shen, Ming-Ming Cheng, and lan Reid and methods described in the paper “Unsupervised Learning of Depth and Ego-Motion from Video” appearing as arXiv preprint arXiv: 1704.07813v2 and by Tinghui Zhou, Matthew Brown, Noah Snavely, and David G. Lowe.

Similarly, pose network 715b (when, e.g., the pose is not determined in parallel with one of the above approaches for network 715a) may be a ResNet “encoder” type network (e.g., a ResNet (18) encoder), with its input layer modified to accept two images (e.g., a 6-channel input to receive image 705a and image 705b as a concatenated RGB input). The bottleneck features of this pose network 715b may then be averaged spatially and passed through a 1×1 convolutional layer to output 6 parameters for the relative camera pose (e.g., three for translation and three for rotation, given the three-dimensional space). In some embodiments, another 1×1 head may be used to extract the two brightness correction parameters, e.g., as was described in the paper “D3VO: Deep Depth, Deep Pose and Deep Uncertainty for Monocular Visual Odometry” appearing as an arXiv preprint arXiv: 2003.01060v2 by Nan Yang, Lukas von Stumberg, Rui Wang, and Daniel Cremers. In some embodiments, each output may be accompanied by uncertainty values 755a or 755b (e.g., using methods as described in in the D3VO paper). One will recognize, however, that many embodiments generate only pose and depth data without accompanying uncertainty estimations. In some embodiments, pose network 715b may alternatively be a PWC-Net as described in the paper “PWC-Net: CNNs for Optical Flow Using Pyramid, Warping, and Cost Volume” available as an arXiv preprint arXiv: 1709.02371v3 by Deqing Sun, Xiaodong Yang, Ming-Yu Liu, and Jan Kautz or as described in the paper “Towards Better Generalization: Joint Depth-Pose Learning without PoseNet” available as an arXiv preprint arXiv: 2004.01314v2 by Wang Zhao, Shaohui Liu, Yezhi Shu, and Yong-Jin Liu.

One will appreciate that the pose network may be trained with supervised or self-supervised approaches, but with different losses. In supervised training, direct supervision on the pose values (rotation, translation) from the synthetic data or relative camera poses, e.g., from a Structure-from-Motion (SfM) model such as COLMAP (described in the paper “Structure-from-motion revisited” appearing in Proceedings of the IEEE conference on computer vision and pattern recognition. 2016 by Johannes L. Schonberger, and Jan-Michael Frahm) may be used. In self-supervised training, photometric loss may instead provide the self-supervision.

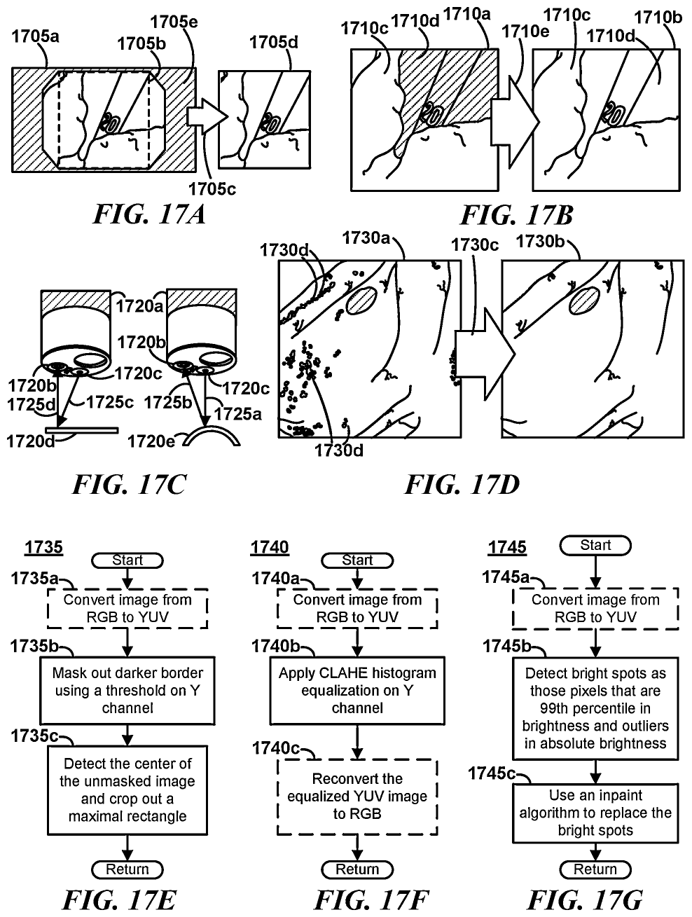

Some embodiments may employ the auto-encoder and feature loss as described in the paper “Feature-metric Loss for Self-supervised Learning of Depth and Egomotion” available as arXiv preprint arXiv: 2007.10603v1 and by Chang Shu, Kun Yu, Zhixiang Duan, and Kuiyuan Yang. Embodiments may supplement this approach with differentiable fisheye back-projection and projection, e.g., as described in the 2019 paper “FisheyeDistanceNet: Self-Supervised Scale-Aware Distance Estimation using Monocular Fisheye Camera for Autonomous Driving” available as arXiv preprint arXiv: 1910.04076v4 and by Varun Ravi Kumar, Sandesh Athni Hiremath, Markus Bach, Stefan Milz, Christian Witt, Clement Pinard, Senthil Yogamani, and Patrick Mäder or as implemented in the OpenCV™ Fisheye camera model, which may be used to calculate back-projections for fisheye distortions. Some embodiments also add reflection masks during training (and inference) by thresholding the Y channel of the YUV images (e.g., using the same methods described herein for landmark recognition in FIGS. 17A-E). During training, the loss values in these masked regions may be ignored and in-painted using OpenCV™ as discussed in the paper “RNNSLAM: Reconstructing the 3D colon to visualize missing regions during a colonoscopy” appearing in Medical image analysis 72 (2021): 102100 by Ruibin Ma, Rui Wang, Yubo Zhang, Stephen Pizer, Sarah K. McGill, Julian Rosenman, and Jan-Michael Frahm.

Given the difficulty in acquiring real-world training data, synthetic data may be used in generating instances of some embodiments. In these example implementations, the loss for depth when using synthetic data may be the “scale invariant loss” as introduced in the 2014 paper “Depth Map Prediction from a Single Image using a Multi-Scale Deep Network” appearing as arXiv preprint arXiv: 1406.2283v1 and by David Eigen, Christian Puhrsch, and Rob Fergus. As discussed above, some embodiments may employ a general-purpose Structure-from-Motion (SfM) and Multi-View Stereo (MVS) pipeline COLMAP implementation, additionally learning camera intrinsics (e.g., focal length and offsets) in a self-supervised manner, as described in the 2019 paper “Depth from Videos in the Wild: Unsupervised Monocular Depth Learning from Unknown Cameras” appearing as arXiv preprint arXiv: 1904.04998v1 by Ariel Gordon, Hanhan Li, Rico Jonschkowski, and Anelia Angelova. These embodiments may also learn distortion coefficients for fisheye cameras.

Thus, though networks 715a and 715b are shown separately in the pipeline 700a, one will appreciate variations wherein a single network architecture may be used to perform both of their functions. Accordingly, for clarity, FIG. 7B depicts a variation wherein a single network 715c receives all the input images 710d (again, ellipsis 760 here indicates that some embodiments may receive more than two images, though one will appreciate that many embodiments will receive only two successive images). As before, such a network 715c may be configured to output 720c, 720d, 720e, 720f the depth prediction 725, pose prediction 730, and in some embodiments, one or more uncertainty predictions 755c, 755d (e.g., determining uncertainty as in D3VO, though one will readily appreciate variations). Separate networks as in pipeline 700a may simplify training, though some deployments may benefit from the simplicity of a single architecture as in pipeline 700b.

### Example End-to-End Data Processing Pipeline—Example Pose and Depth Pipeline—Example Training

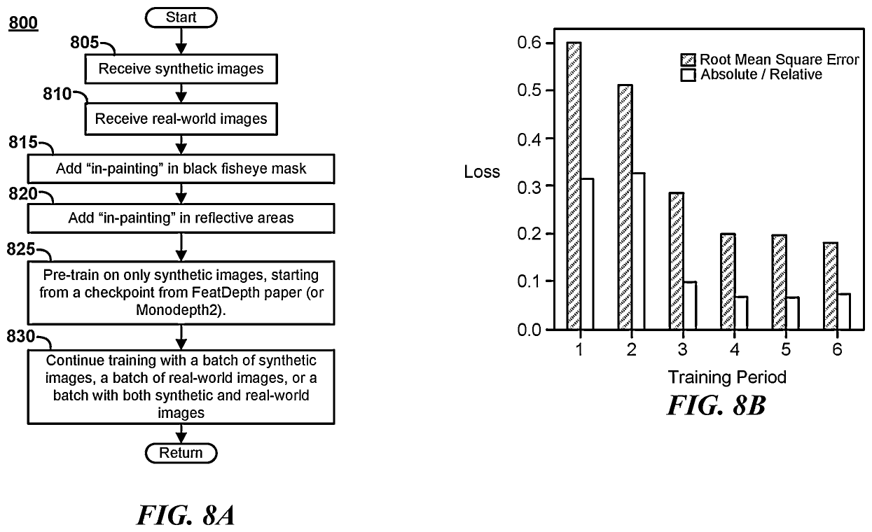

FIG. 8A is a flow diagram illustrating various operations in an example neural network training process 800, e.g., for training each of networks 715a and 715b. At block 805 the system may receive any synthetic images to be used in training and validation. Similarly at block 810, the system may receive the real world images to be used in training and validation. These datasets may be processed at blocks 815 and 820, in-painting reflective areas and fisheye borders (e.g., using the same methods described herein for landmark recognition in FIGS. 17A-E). One will appreciate that, once deployed, similar preprocessing may occur upon images not already adjusted in this manner.

At block 825 the networks may be pre-trained upon synthetic images only, e.g., starting from a checkpoint in the FeatDepth network of the “Feature-metric Loss for Self-supervised Learning of Depth and Egomotion” paper or the Monodepth2 network of the “Digging Into Self-Supervised Monocular Depth Estimation” paper referenced above. Where FeatDepth is used, one will appreciate that an auto-encoder and feature loss as described in that paper may be used. Following this pre-training, the networks may continue training with data comprising both synthetic and real data at block 830. In some embodiments, COLMAP sparse depth and relative camera pose supervision may be here introduced into the training.

FIG. 8B is a bar plot depicting an exemplary set of training results for the process of FIG. 8A.

### Example Fragment Management

As discussed with respect to process 500, the depth frame consolidation process may be facilitated by organizing frames into fragments (e.g., at block 585a) as the camera encounters sufficiently distinct regions, e.g., as determined at block 580. An example process for making such a determination at block 580 is depicted in FIG. 9A. Specifically, after receiving a new depth frame at block 905a (e.g., as generated at block 555) the computer system may apply a collection of rules or conditions for determining if the depth frame or pose data is indicative of a new region (precipitating a transition to block 905e, corresponding to a “YES” transition from block 580) or if the frame is instead indicative of a continuation of an existing region (precipitating a transition to block 905f, corresponding to a “NO” transition from block 580).

In the depicted example, the determination is made by a sequence of conditions, the fulfillment of any one of which results in the creation of a new fragment. For example, with respect to the condition of block 905b, if the computer system fails to estimate a pose (e.g., where no adequate value can be determined, or no value with an acceptable level of uncertainty) at either block 550 or at block 555, then the system may begin creation of a new fragment. Similarly, the condition of block 905c may be fulfilled when too few of the features (e.g., the SIFT or ORB features) match between successive frames (e.g., at block 545), e.g., less than an empirically determined threshold. In some embodiments, not just the number of matches, but their distribution may be assessed at block 905c, as by, e.g., performing a Singular Value Decomposition of the depth values organized into a matrix and then checking the two largest resulting eigenvalues. If one eigenvalue is not significantly larger than the other, the points may be collinear, suggesting a poor data capture. Finally, even if a pose is determined (either via the pose from block 550 or from block 555), the condition of block 905d may also serve to “sanity” check that the pose is appropriate by moving the depth values determined for that pose (at block 555) to an orientation where they can be compared with depth values from another frame. Specifically, FIG. 9B illustrates an endoscope moving 970 over a surface 985 from a first position 975a to a second position 975b with corresponding fields of view 975c and 975d respectively. One would expect depth values between the region 980 to overlap, as shown by the portion 980 of the surface 985. The overlap in depth values may be verified by moving the values in one capture to their corresponding position in the other capture (as considered at block 905d). A lack of similar depth values within a threshold may be indicative of a failure to acquire a proper pose or depth determination.

One will appreciate that while the conditions of blocks 905a, 905b, and 905c may serve to recognize when the endoscope travels into a field of view sufficiently different from that in which it was previously situated, the conditions may also indicate when smoke, biomass, body structures, etc. obscure the camera's field of view. To facilitate the reader's comprehension of these latter situations, an example circumstance precipitating such a result is shown in the temporal series of cross-sectional views in FIG. 9C. Endoscopes may regularly collide with portions of the body interior during an examination. For example, initially at time 910a the colonoscope may be in a position 920a (analogous to the previous discussion with respect to FIGS. 4A-C) with a field of view suitable for pose determination. Unfortunately, patient movement, inadvertent operator movement, etc., may transition 910d the configuration to the new state of time 910b, where the camera collides with a ridge wall 915a resulting in a substantially occluded view, mostly capturing a surface region 915b of the ridge. Naturally, in this orientation 920b, the endoscope camera captures few, if any, pixels useful for any proper pose determination. When the automated examination system or operator recovers 910e at time 910c the endoscope may again be in a position 920c with a field of view suitable for making a pose and depth determination.

One will appreciate that, even if such a collision only occurs over the course of a few seconds or less, the high frequency with which the camera captures visual images may precipitate many new visual images. Consequently, the system may attempt to produce many corresponding depth frames and poses, which may themselves be assembled into fragments in accordance with the process 500. Undesirable fragments, such as these, may be excluded by the process of global pose graph optimization at block 585b and integration at block 570. Fortuitously, this exclusion process may itself also facilitate the detection and recognition of various adverse events during procedures.

Specifically, FIG. 9D is a schematic collection of fragments 925a, 925b, and 925c. Fragment 925a may have been generated while the colonoscope was in the position of time 910a, fragment 925b may have been generated while the colonoscope was in the position of time 910b, and fragment 925c may have been generated while the colonoscope was in the position of time 910c. As discussed, each of fragments 925a, 925b, and 925c may include an initial keyframe 930a, 930e, and 930f respectively (here, the keyframe is the first frame inserted into the fragment). Thus, for clarity, the first frame of fragment 925a is keyframe 930a, frame 930b was the next acquired frame, and so on (intermediate frames being represented by ellipsis 930d) until the final frame 930c is reached. During global pose estimation at block 585b, the computer system may have recognized sufficient feature (e.g., SIFT or ORB) or depth frame similarity between keyframes 930a and 930f that they could be identified as depicting connected regions of depth values (represented by link 935c). This is not surprising given the similarity of the field of view at times 910a and 910c. However, the radical character of the field of view at time 910b, makes keyframe 930e too disparate from either keyframe 930a or 930f to form a connection (represented by the nonexistent links 935a and 935b).

Consequently, as shown in the hypothetical graph pose network of FIG. 9E, viable fragments 940a, 940b, 940c, 940d, 940e and 925a, and 925c may form a network with reachable nodes based upon their related keyframes, but fragment 925b may remain isolated. One will appreciate that frame 925b may coincidentally match other frames on occasion (e.g., where there are multiple defective frames resulting from the camera pressed against a flat surface, they may all resemble one another), but these defective frames will typically form a much smaller, isolated (or more isolated) network from the primary network corresponding to capture of the internal body structure. Consequently, such frames may be readily identified and removed from the model generation process at block 570.

Though not shown in FIG. 9D, one will appreciate that, in addition to depth values, each frame in a fragment may have a variety of metadata, including, e.g., the corresponding visual image(s), estimated pose(s) associated therewith, timestamp(s) at which the acquisition occurred, etc. For example, as shown in FIG. 9F, fragments 950a and 950b are two of many fragments appearing in a network (the presence of preceding, succeeding, and intervening fragments represented by ellipses 965a, 965c, and 965b, respectively). Fragment 950a includes the frames 950c, 950d, and 950f (ellipsis 950e reflecting intervening frames) and the first temporally acquired frame 950c is designated as the keyframe. From the frames in fragment 950a one may generate an intermediate model such as a TSDF representation 955a (similarly, one may generate an intermediate model, such as TSDF 955b, for the frames of fragment 950b). With such intermediate TSDFs available, integration of fragments into a partial or complete model mesh 960 may proceed very quickly (e.g., at block 570 or integration 680), which may be useful for facilitating real-time operation during the surgery.

### Example Processing Pipeline Variations

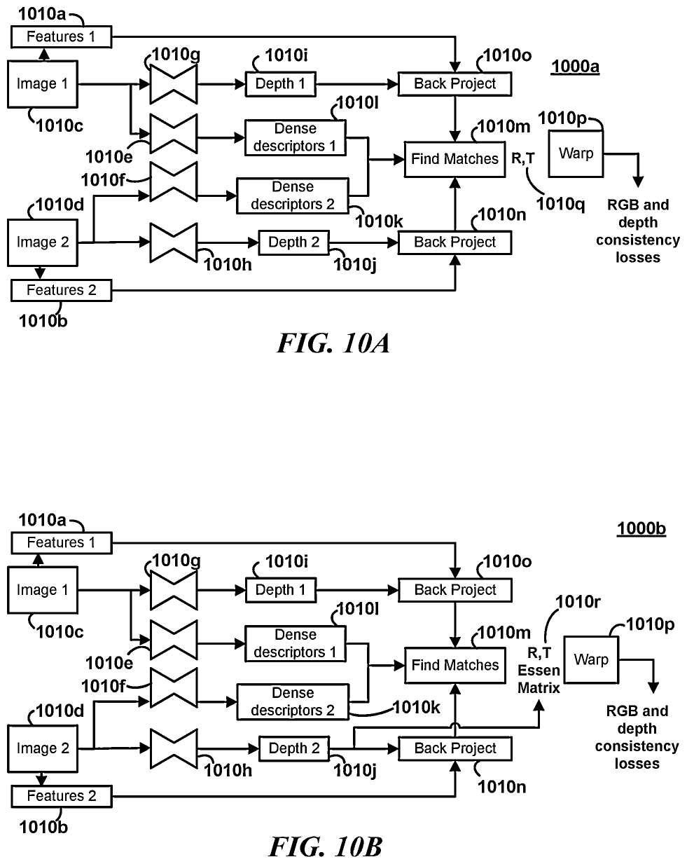

One will appreciate numerous variations for the architectures and processes described herein. For example, FIGS. 10A and 10B depict variant processing pipelines for performing the operations of process 500 and processing pipeline 600. With reference to FIG. 10A, as before, two temporally successive images (Image 1 1010c and then Image 2 1010d) may be provided to the processing pipeline 1000a. The computer system may produce features 1010a, 1010b such as SIFT, ORB, etc., as discussed above, for Image 1 1010c and Image 2 1010d respectively. The images may also, in parallel or in series, be separately provided to neural networks, e.g. UNet-type architectures, 1010e, 1010f, 1010g, and 1010h and shown. Network 1010g may generate predicted depth values 1010i for Image 1 1010c using the methods described and referenced herein, and network 1010e may generate dense descriptors 10101 for Image 1 1010c (“dense descriptors” here referring to pixel based features analogous to SIFT, ORB, etc. but generated using a neural network). Similarly, network 1010h may generate predicted depth values 1010j for Image 2 1010c, and network 1010f may generate dense descriptors 1010k for Image 2 1010d. The networks 1010e and 1010f may be trained to learn a descriptor for each pixel that takes into account the global context of the input image, e.g., as described in the paper “Towards Better Generalization: Joint Depth-Pose Learning without PoseNet” referenced above. For example, a dense descriptor may be generated by training a network having CNN layers to recognize features which are combinations of SIFT, ORB, etc. selected specifically for the endoscopic context at hand.

Each of depth values 1010i and 1010j may be back projected as indicated by blocks 10100 and 1010n. One will appreciate that back projection here refers to a process of “sending” (i.e., transforming) depth values from the image pixel coordinates to the 3D coordinate system of the final model. This may facilitate the finding of matches between each of the back projected values and dense descriptors at block 1010m. The matches may indicate rotation and translation operations 1010q suitable for warping one image to the other as indicated by block 1010p. One will appreciate that warping is the process of interpolating and projecting one image upon another image, e.g., as described in the “Digging Into Self-Supervised Monocular Depth Estimation” paper referenced above, as well as in the paper “Unsupervised Learning of Depth and Ego-Motion from Video” also referenced above. Thus, R and T here refer to rotation and translation from the relative pose for the warp. Having identified warp 1010p, RGB and depth consistency losses may be determined and used for refining the determination of the relative coarse pose between the images 1010a, 1010d.

Process pipeline 1000b of FIG. 10B may include many of the same relations and components as the pipeline 1000a. As indicated, however, an additional connection from the second set of depth values 1010j may facilitate the determination of Fundamental/Essential Matrix (a 3×3 matrix relating corresponding points between the images) in addition to the rotation and translation operations 1010r. Determining a warp from these values may likewise facilitate RGB and depth consistency losses may be inferred, which may themselves facilitate a relative coarse pose determination between the images.

### Example Model Hole Identification

Once a model has been generated following integration (e.g., after block 570, or after integration 680, when a partially complete model 690a or fully complete model is available) one will appreciate that portions of the model may still omit regions of the internal body structure, possibly regions not intended to be omitted during the examination. These omitted regions, referred to herein as “holes”, may manifest as lacunae in the vertex mesh of the completed or partially completed model. While some embodiments may simply ignore holes, focusing upon other of the features disclosed herein, holes may reflect significant oversights, containing, e.g., polyps and adenomas, resulting from sharp camera movement, occluded regions of the internal body structure, regions the operator failed to examine, etc.

Accordingly, some embodiments may recognize holes by directly inspecting the vertex distribution of the model or by using a machine learning architecture, such as a neural network, trained to recognize missing portions of models. In some embodiments, rather than recognizing the holes directly, the computer system may interpolate across lacunae in the model, or apply a neural network trained for that purpose (e.g., a network trained to perform inpainting on a three-dimensional model) and subtract the original from this in-filled result as described herein. One will appreciate that a neural network may be trained, e.g., from segmented portions of a colon from computerized tomography (CT) scans. For example, such scans may be used to create a “complete” 3D model of the internal body structure, from which one may readily introduce numerable variations by removing various portions of the model (e.g., deliberating removing some portions to mimic occlusions, blurred data captures, and other defects occurring during an examination). This corpus of incomplete models may then be used to train the neural network (e.g., using a three dimensional mask) to predict in-fill meshes so as to again achieve the original, complete model (for clarity, the network's loss being, e.g., a difference between the predicted in-filled mesh model and the original mesh model).

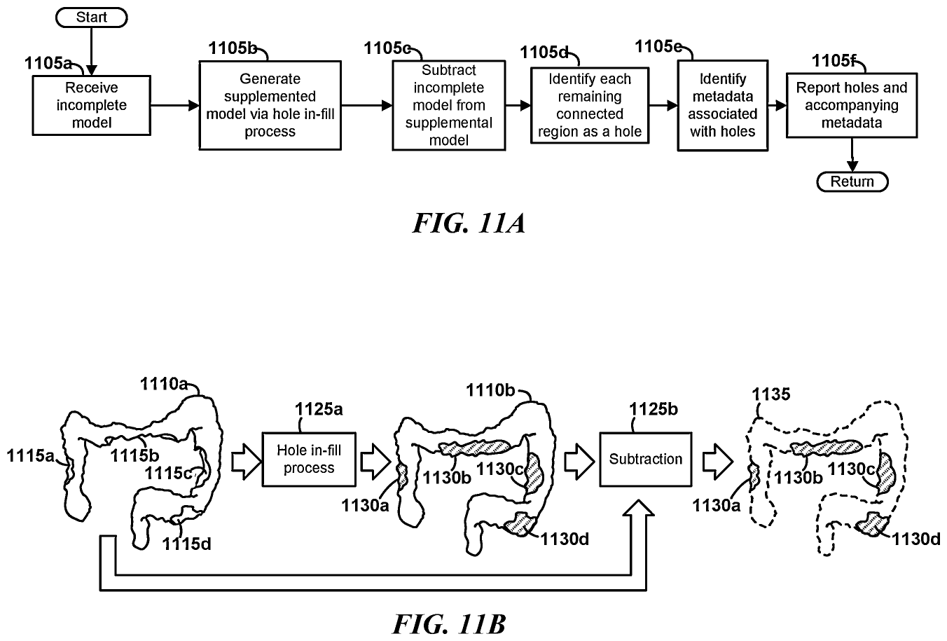

For example, with reference to FIGS. 11A and 11B, during or following a surgical examination, at block 1105a a computer system may receive the incomplete model 1110a (i.e., a model with holes). As shown, the model 1110a may include holes 1115a, 1115b, 1115c, and 1115d resulting from interior occlusions, occluding biomass, regions not viewed with the endoscope, regions viewed so quickly that they were blurred, etc. for which no second pass was made to remedy the lacunae. At block 1105b, the computer system may generate a supplemented model 1110b using a hole in-fill process 1125a. As mentioned, hole in-fill process 1125a may be accomplished using a trained in-fill neural network (e.g., a network employing a three dimensional flood-fill mask, a 3DCNN, e.g., a 3D-UNet, as discussed in greater detail herein, etc.),), by interpolating between vertices surrounding a lacuna, etc. Such supplementation may result in a model with various in-filled regions 1130a, 1130b, 1130c, 1130d.

At block 1105c the computer system may subtract 1125b the original incomplete model 1110a from the supplemented model 1110b (e.g., removing vertices at the same, or nearly the same, locations in each model). The resulting isolated regions 1130a, 1130b, 1130c, 1130d may then be identified as holes at block 1105d (one will appreciate that outline 1135 is shown merely to facilitate the reader's understanding and does not itself represent any remaining structure following the subtraction). Vertices in model 1110a nearest these regions may then be identified as the outlines or boundaries of the holes.

In some embodiments, the process may continue to blocks 1105e and 1105f wherein available metadata from regions near the holes is respectively identified and reported (e.g., from discarded fragments temporally near fragments associated with the hole boundary vertices). For example, timestamps and visual images associated with discarded fragments acquired near fragments used to generate regions of the model near the hole may be associated with the hole (as when, e.g., bile, blood, feces, or other biomass obscured the view, the system may identify those images, their timestamps, and the hole(s) with which they are associated). Thus, metadata from removed fragments (e.g., those isolated from the pose network) or metadata from neighboring fragments contributing to the incomplete model 1110a may each be used to identify information related to each hole.

When applied in real-time during a surgical operation, the system may direct the operator to these holes once they are identified. Hole identification may be applied only at a distance from the current point of inspection, so as to alert the operator only to defects in regions previously considered by the operator. As discussed herein, a quality coverage score may be calculated following, or during, the procedure, to also provide the operator with feedback (determined, e.g., as the ratio of the surface areas or ratio of volumes of meshes 1110a and 1110b). This may facilitate a “per-segment” (e.g., a portion of the model generated from one or more fragments) indication of coverage quality, as a distinct score may be calculated for each of several discrete portions of previously considered regions in the model. Such real-time feedback may encourage the operator to revisit a region they previously omitted, e.g., revisiting a region during extraction, which the operator overlooked during insertion.

### Example Model Supplementation Operations

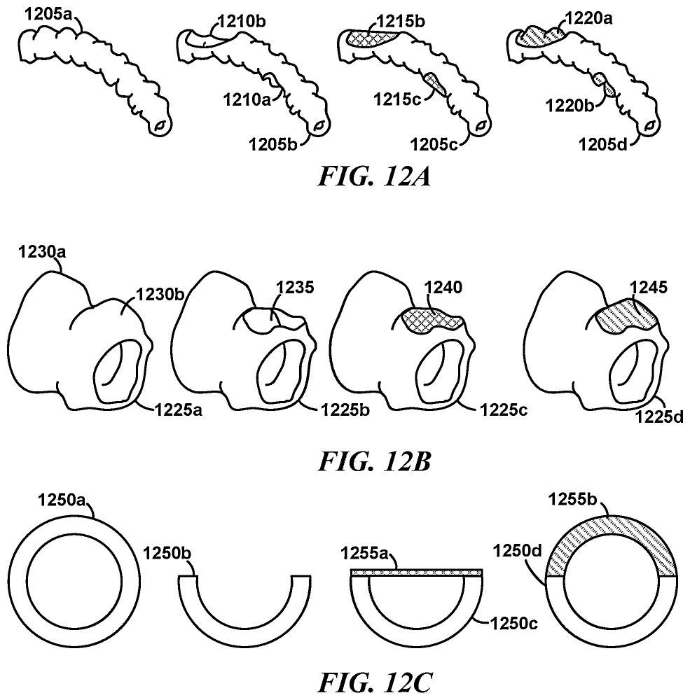

While real-time or post-process infilling of the model at block 1105b, using a neural network trained upon three-dimensional structures created from CT scans may suffice in some embodiments, one will appreciate that for the purposes of hole identification, a variety of in-filling techniques may suffice, some requiring less training or processing power, but perhaps just as suitable depending upon the fidelity with which the hole is to be recognized and compensated for. For example, FIGS. 12A, 12B, and 12C each provide a series of schematic representations of three-dimensional computer-generated model in-filling results for a portion of a colon, a haustra pair, and a cylinder, respectively.

The complete, or ideally reconstructed models are shown with meshes 1205a, 1225a, 1250a (again, for clarity, the reader will appreciate that the cylinder of FIG. 12C is viewed in cross-section down one of its ends). As indicated, the haustra pair includes two lobes 1230a, 1230b. Versions of these models with holes 1210a, 1210b, 1235 are shown in modified models 1205b, 1225b, and 1250b. For clarity, in the case of FIG. 12C the top half of the cylinder has been removed to create the incomplete mesh 1250b. Similarly, the reader will appreciate that hole 1235 has revealed the interior of the haustrum. Naturally, in some embodiments, as only the interior of the model was captured by the endoscope camera, the mesh may comprise an inner and an outer surface, the inner textured based upon the collected visual images, and the exterior textured with a vertex mesh coloring, or an arbitrary texture.

Via a vertex in-fill or interpolation algorithm, one will appreciate that the holes of the incomplete models may be in-filled as shown in supplemented models 1205c, 1225c, and 1250c. Specifically, supplemented model 1205c includes in-filled portions 1215b, and 1215c, the model 1225c includes in-filled portion 1240, and the half cylinder 1250c is completed with in-filled portion 1255a (though shown as a flat plane here, one will appreciate that some vertex or plane-based in-fill algorithms may likewise result in each end of the cylinder being likewise covered with the in-filled portion). Because more simple in-fill algorithms do not attempt to estimate the original structure, they may result in flat (as shown) or idealized interpolations over the holes. Such methods may suffice where the system merely wishes to identify the existence of a hole, without attempting to reconstruct the original model (e.g., where a high fidelity coverage score is not desired).

In contrast to these supplemented models, the in-fill approach used to create supplemented models 1205d, 1225d, and 1250d may create in-fill portions 1220a, 1220b, 1245, and 1255b more closely resembling the original structure. In-filling with a neural network trained upon modified models generated from CT scans, as discussed above, may produce these higher fidelity in-fill regions (though, in some situations, e.g., to simplify scoring, one may prefer to train to in-fill with the lower fidelity portions, e.g., 1240, 1255a). Such higher fidelity may result in improved downstream operations. For example, the choice of in-fill method may affect a subsequent estimation of the structure's total volume or surface area. High fidelity in-fills may thus precipitate more accurate determinations of coverage quality, omission risks (e.g., the unseen surface area in which a cancerous growth may reside), etc. when the incomplete model is compared to the supplemented or complete model.

### Example Model Supplementation Assessments

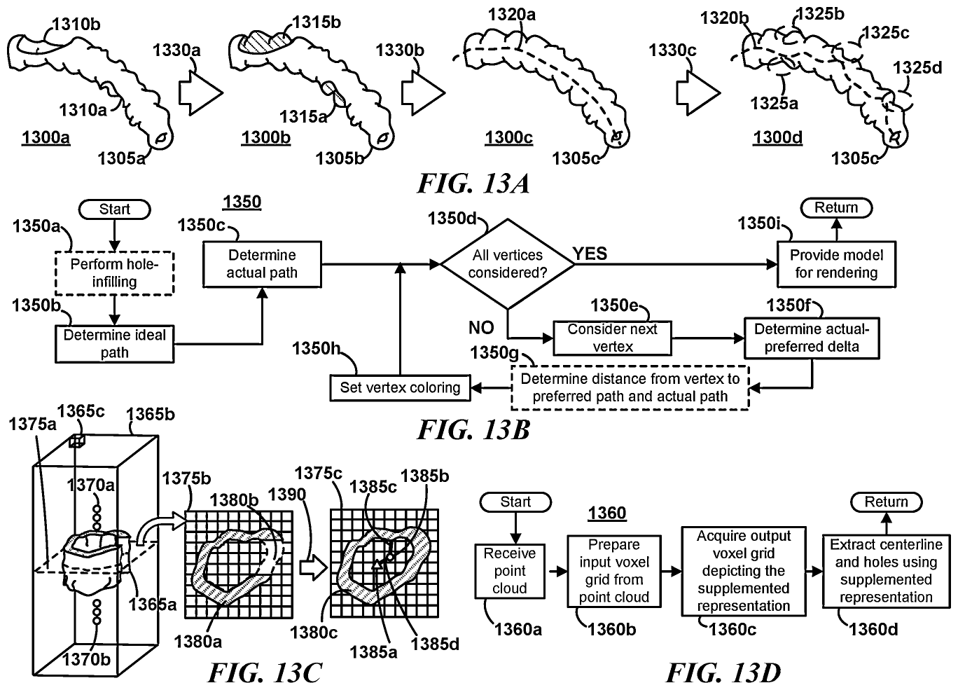

As mentioned, comparison of the volume or surface area of models 1110a and 1110b may facilitate a quick metric for assessing the comprehensiveness of the surgical examination. Additional metrics assessing the quality of the examination may likewise be inferred from the examination path as determined, e.g., from successive poses of the endoscopic device. Specifically, FIG. 13A is a schematic representation of a plurality of model states prepared for centerline determination and examination path assessment as may be implemented in some embodiments.

Initially, as shown in state 1300a the system may receive or generate a model 1305a (e.g., the same as model 1110a), which may contain several holes 1310b, 1310a. Following an in-filling process 1330a, as shown in state 1300b, the system may now have access to a supplemented model 1305b with supplemental regions 1315a and 1315b (e.g., the same as model 1110b). One will appreciate that the supplemented model 1305b may likewise have been generated in a previous process. Alternatively, in some embodiments, the model 1305a may be assumed to be sufficiently complete and used without performing any hole in-filling.

In either event, once the system has access to the model 1305c upon which centerline detection will be performed, the system may perform centerline detection 1330b as shown in state 1300c. The centerline 1320a may be determined as the “ideal” path through the model for the procedure under consideration. For example, during a colonoscopic examination, the path through the model which most avoids the model's walls (i.e., the edges of the colon) may be construed as the “ideal” path. One will appreciate that not all examinations may involve passage through a cylindrical structure in this manner. For example, in some laparoscopic surgeries, the “ideal” path may be any path within a fixed distance from the laparoscopic surgery entry point. Here, the path may be identified, e.g., based upon an average position of model points, from a numerical force-directed estimation away from the model walls, etc.

With the “ideal” path identified, the system may consider 1330c the actual path 1320b taken during the examination as shown in state 1300d, e.g., by concatenating the inferred poses in each of the fragments (e.g., just the keyframes) relative to the global coordinates of the captured mesh. The actual path 1320b may then be compared with the ideal path 1320a as a reference for assessing the character or quality of actual path 1320b. In this depicted example, the actual path 1320b has most clearly deviated from the ideal path 1320a where it approaches the walls of the model at regions 1325a, 1325c, and 1325d. When the model 1305c is rendered (again model 1305c may be either of model 1305a or model 1305b), the rendering pipeline may be adjusted based upon the comparison between the ideal path 1320a and actual path 1320b. For example, the vertex shading or texturing of the interior or exterior planes of the rendering of the model 1305c may be adjusted to indicate relations between the ideal and actual paths. Analogous to a heat map, regions 1325a, 1325c, 1325d, may be subject to more red vertex colorings, while the remainder of the model where the actual path 1320b more closely tracks the desired path 1320a may receive a more green vertex coloring (though one will appreciate a variety of suitable colorings and palettes). Such colorings may quickly and readily alert the reviewer to regions of less than ideal traversal. In some embodiments, all vertices within a fixed distance of the deviation may be marked as “poor”, i.e., both of regions 1325a and 1325b of the model may be vertex shaded a more red color. In some embodiments, however, the system may instead consider the distance between the model and the actual path in its rendering, which would result instead in rendering the region 1325b green and region 1325a red (again, recognizing that other colorings may be performed).

For clarity, FIG. 13B is a flow diagram illustrating various operations in an example process 1350 for determining and representing path quality to a reviewer. Specifically, at block 1350a, the system may perform the hole in-filling process discussed above (e.g., the operation 1330a), though, again, one will appreciate that in some embodiments the original model may be considered. At block 1350b the system may determine a preferred ideal path (e.g., the model centerline 1320a) as discussed above and may determine the actual path (e.g., the path 1320b) as discussed above at block 1350c.

Subsequently, when rendering the model, if the quality score is to be represented by vertex shading (wherein the coloring of planes of the mesh during rendering is interpolated based upon the coloring of the plane's constituent vertices), then at blocks 1350d and 1350e the system may iterate over the vertices of the model to be rendered and determine the delta between the actual and preferred path at block 1350f relative to the vertex position. As a single region may be traversed multiple times during the surgery, one will appreciate that these operations may be applied for the specific portion of the playback being presently considered (that is, the vertex shading of the model may be animated and may change over the course of the playback). In some embodiments, however, the vertex shading may be fixed throughout the playback, as, e.g., when the delta determined at block 1350f is the median, average, worst-instance, etc. delta value for that region for all the instances in which the actual path 1320b traversed the region associated with the vertex of block 1350e. In some embodiments, at block 1350g, the system may determine the distance from the vertex of block 1350e to the nearest point upon the preferred path. By considering the vertex's relation to the delta determined at block 1350f, the system may “normalize” the disparity. For example, where there is a large delta at block 1350f and the actual path is very near the vertex at block 1350g, then then vertex may be marked for coloring at block 1350h in accordance with its being associated with a very undesirable deviation from the preferred path. However, in some situations, even a large delta may be less relevant to certain portions of the model. For example, if desired, when a deviation occurs, one may wish to distinguish between the region 1325a and the region 1325b. Vertices in each region may both be near the delta between the actual and preferred paths, but region 1325a may be colored differently from region 1325b to stress that the delta vector is approaching region 1325a and pointing away from region 1325b (one will appreciate that some embodiments may analogously dispense with the delta calculation and instead simply determine the appropriate coloring based upon each vertex's distance to the portion or portions of the actual path closest to that vertex). This distinction may provide a more specific depiction of the nature of the deviation, as when, e.g., an operator habitually travels too close to a lower portion of the model or where an automated system overfit upon its navigation data repeatedly over adjusts in a particular direction.

Once all vertices have been considered, the model may be rendered at block 1350i. Where the rendering is animated over the course of playback, one will appreciate that the process 1350 may be applied to a region of the model around the current position of the distal tip, while the rest of the model is rendered in a default color (the same may be done for a highlighted region under a cursor as disclosed herein).

### Example Model Preparations for Supplementation and Formatting

As discussed above, one will appreciate a variety of methods for supplementing a model containing holes, as well as for determining a centerline. Various experiments have demonstrated, for example, that application of a 3D-UNet type architecture to a voxel formatted model input produced especially good in-filling and centerline prediction results, facilitating much more granular coverage score determinations (comparing the volume or surface area of the supplemented model with the incomplete original). Specifically, one may modify the 3D-UNet's inputs to receive a three-dimensional voxel grid of all or a section of the internal body structure, with each voxel assigned a value depending upon the presence or absence of internal body structure (e.g., assigning 0 to a voxel where the colon sidewall is present and 1 where the colon is absent). Some embodiments may use networks similar to those described in the paper “Shape Completion using 3D-Encoder-Predictor CNNs and Shape Synthesis” appearing as arXiv preprint arXiv: 1612.00101v2 and by Angela Dai, Charles Ruizhongtai Qi, and Matthias Nießner, in the paper “GRNet: Gridding Residual Network for Dense Point Cloud Completion” appearing as arXiv preprint arXiv: 2006.03761v4 and by Haozhe Xie, Hongxun Yao, Shangchen Zhou, Jiageng Mao, Shengping Zhang, and Wenxiu Sun, or in the paper “3D U-Net: Learning Dense Volumetric Segmentation from Sparse Annotation” available as arXiv preprint arXiv: 1606.06650v1 and by Özgün çiçek, Ahmed Abdulkadir, Soeren S. Lienkamp, Thomas Brox, and Olaf Ronneberger. The neural network's output may be similarly modified to output a voxel grid with values indicating where original and in-filling internal body structure appear, as well as where the centerline (if applicable) appears (e.g., values of 0 for voxels depicting colon, values of 1 for voxels depicting the centerline, and intermediate values between 0 and 1 for all other voxels, e.g., as determined using Equations 1, 2, and 3 described herein; naturally, in some embodiments, one may ignore the voxel-based centerline described here and instead use the approach of FIG. 13A at 1330c for determining the centerline). One will appreciate that this output voxel representation may then be converted to any desired model form (e.g., a 3D mesh using convex hulls around the voxels with values of 0) if the voxel format is not preferred for subsequent processing. Such an voxel-based approach may be especially suitable where CT scans of (synthetic or real-world) internal body structure data are available for training the neural network (as mentioned, one can easily manually excise portions of the internal body structure model in this data to mimic holes as they may occur in training).

For clarity, FIG. 13C depicts a volumetric representation 1365a, e.g., a CT scan data or depth data, of a portion of an internal body structure, in this example a colon (ellipses 1370a and 1370b indicating the continuation of the colon above and below). As indicated the colon portion is not complete, assuming an “extruded C” shape where a portion of the model is missing (e.g., being deliberately removed for training or inadvertently removed during a live data capture). One will appreciate that volumetric representation 1365a may be readily formatted to a voxel representation by mapping volumetric representation 1365a to a three-dimensional grid, e.g., by dividing the region 1365b enclosing the representation into cube sub-divisions (i.e., voxels) like cube 1365c. Thus, if a two-dimensional plane 1375a were taken of the voxels as shown, a grid-like two-dimensional representation 1375b of the voxel values may be produced.

Again, for clarity with reference to two-dimensional representation 1375b, various of the voxel values may indicate the presence of the colon 1380a, though values within region 1380b, where the hole exists, will be as empty as the other of the non-colon voxels (one will appreciate that while CT scan data may present a colon wall with substantial thickness, depth data may instead only provide the surface wall, and so the depth data may be extruded accordingly, the CT scan data adjusted to be commensurate with camera-captured data, etc.). Again, while represented here in two-dimensions to facilitate understanding one will appreciate that the voxels in plane 1375a representation 1375b are three-dimensional voxel cells.

Submitting all or a portion of the region 1365b to a machine learning architecture, such as a 3D-UNet, may produce 1390 a supplemented voxel representation. For clarity, the counterpart representation 1375c to the representation 1375b is shown in lieu of a complete three-dimensional voxel model. In this output, the voxels associated with the colon 1380c may now include those voxel locations previously corresponding to the missing region 1380b. It may also be possible to discern the relations between the voxels and a centerline 1385a (again, though centerline 1385a appears as a point in the two-dimensional representation 1375c one will appreciate that the centerline is a line, or tube, in the full three-dimensional space). To train a machine learning architecture to produce 1375b this supplemented voxel representation, target voxel fames with the “correct” voxel values may be used to assess the loss (e.g., the L2 loss) between the machine learning architecture's prediction and this proper result at each training iteration (e.g., to perform the appropriate backpropagation).

For example, the following schema may be used to assess the “correct” voxel values for target voxel volume. Given a voxel associated with a three-dimensional point 1385b, the voxel's value may be determined based upon a first metric incorporating the distance 1385c from the voxel with point 1385b to the nearest voxel associated with the colon and a second metric incorporating the distance 1385d from the voxel with point 1385b to the centerline 1385a (though each of the distances distance 1385c and distance 1385d are shown as lines in a plane here to facilitate understanding, one will appreciate that the distances are taken in the three-dimensional space of the voxel volume, thus, the distance vectors need not be in the same plane). The first metric is referred to herein as segᵥₐₗ and may be calculated as shown in Equation 1:

$${seg}_{val} = tan h ( 0 \text{.2} * point\_to {\_se g}_{dist} ) \qquad ( 1 )$$

where point_to_seg_{dist} is the distance 1385c (determined in three dimensions).

Similarly, the second metric is referred to herein as centerlineᵥₐₗ and may be calculated as shown in Equation 2:

$${centerline}_{val} = tan h ( 0.1 * {centerline}_{dist} ) \qquad ( 2 )$$

where centerline_{dist} is the distance 1385d (again, determined in three dimensions). One will appreciate that the scaling values 0.2 and 0.1 in Equations 1 and 2, respectively, are here selected based upon the voxel dimensions in use and that alternative scaling values may be appropriate with a change of dimensions. Similarly, the choice of the hyperbolic tangent is here used to enforce a floor and ceiling upon the values in the range 0 to 1. Naturally, one will appreciate other suitable methods for achieving a floor and ceiling, e.g., an appropriately modified logit function, coded operations, etc.

The segᵥₐₗ and centerlineᵥₐₗ metric values may then be related to determine the voxel with point 1385b's value (referred to herein as voxel_{score}), e.g., as shown in Equation 3:

$${voxel}_{score} = \frac{{seg}_{dist}}{{seg}_{dist} + {centerline}_{dist}} \qquad ( 3 )$$

Accordingly, one will appreciate that in the target voxel volume for determining the loss during training, or in the voxel volume output by the machine learning architecture once the machine learning architecture is properly trained, voxels very far from and outside the colon may receive a voxel_{score} of approximately 0.5. In contrast, voxels within the colon may have voxel_{score} values of approximately 0. Finally, voxels within the colon and containing (or very near) the centerline, may have voxel_{score} values of approximately 1. This distribution of voxel_{score} scores facilitates a variety of benefits, including providing a representation suitable for training a machine learning architecture, providing a representation facilitating conversion between the voxel and mesh formats, providing a representation suitable for quickly assessing a distal tip path quality score (e.g., by summing the voxel scores through which the tip passes), and providing a representation readily facilitating identification of the centerline (which may obviate the need for centerline determination from a mesh as discussed elsewhere herein). This approach may also facilitate training to recognize centerlines specific to different surgeries and body structures (rather than, e.g., naively always identifying the middle point in the model segment as the centerline).

Thus, for clarity, during training or inference the voxel representations of the depth values may be prepared and applied in accordance with the process 1360 of FIG. 13D. At block 1360a, a computer system may receive a point cloud representation of the internal body structure, e.g., a colon (e.g., CT scan data, post integration data from a camera, etc.). As discussed, the system may convert the point cloud to a voxel representation at block 1360b (e.g., by subdividing the region 1365b into voxel “cubes”, like cube 1365c). As mentioned, such a resulting three-dimensional grid may be a binary voxel grid, wherein voxels associated with the colon receive a 0 value and non-colon portions receive a 1.

During training, this voxel grid may be copied: one copy modified (e.g., portions removed) to serve as a training input; and the other copy's values converted to the corresponding voxel_{score} values so that it may serve as a “true positive” upon which to assess the machine learning architecture's loss during training (e.g., to assess the machine learning architecture's success in determining correct voxel_{score} from the first copy in accordance with the methodology descried above).

At block 1360c, the computer system may acquire a voxel volume output from the machine learning architecture, depicting the supplemented representation with voxel_{score} value. From this output, at block 1360d, the system may determine a centerline position (e.g., based on the voxels with voxel_{score} values at or near 1), the supplemented model analogous to model 1305b (e.g., applying a convex hull around voxels having voxel_{score} values at or near 0), and the holes (by subtracting the original model from the supplemented model, as described herein).

### Landmark Overview

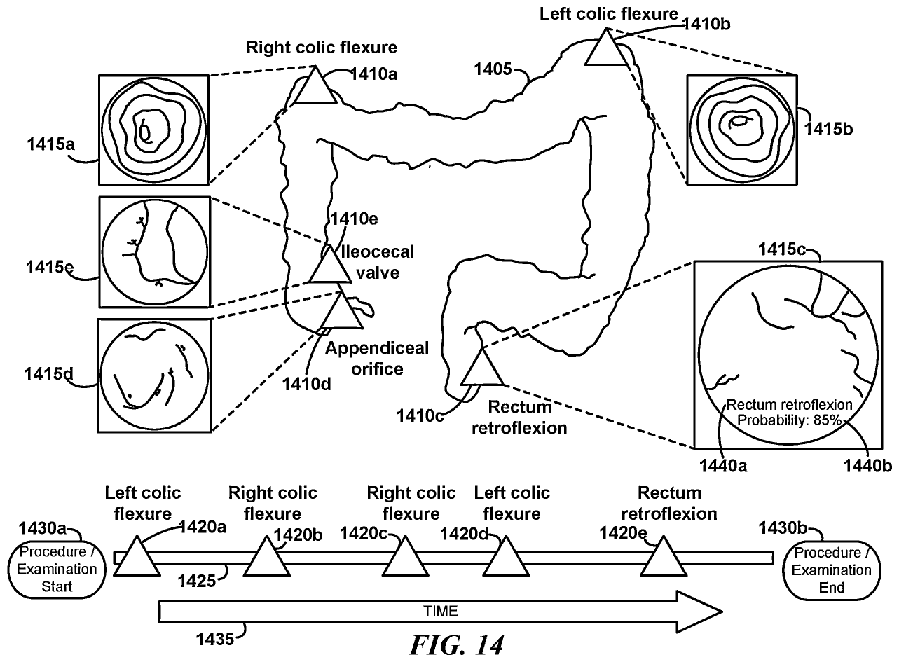

FIG. 14 is schematic depiction of example structural landmarks appearing in an example internal body structure (here a large intestine 1405), as may be occur in some embodiments. Specifically, an internal body structure may include a number of regions and events relevant to surgery, navigation, subsequent quality examination, etc. Recognizing these structures or events (collectively referred to herein as “landmarks”) may provide contextual information and other benefits, including identifying a current location of the examination device (which may be useful to corroborate data from other spatial determination assets such as encoders, RF transponders, etc.), the state of the examination of a surgery (e.g., how far the colonoscope has advanced to its destination), structural variations between such landmark locations (as patient anatomy may vary considerably from the “idealized” relative location of organ structures), useful points of division between stages of a surgical examination (e.g., when considering the surgical examination data offline, or when performing task identification), etc. Thus, landmarks may facilitate, e.g., quality measurements for real-time or offline feedback, or as metadata documentation to the surgical data to facilitate future review of the surgical data. Example structural landmarks in the colonoscopic context include, e.g.: the Ileocecal Valve; the Appendiceal Orifice; the Cecum; the Terminal Ileum; and a Polyp. Naturally, some event landmarks, such as a retroflexion operation, may connote both spatial locations and activity events. Video frames with high probability detections may be suitable as indicative “snapshots” of that point in the procedure. For example, if a sequence of video images during a procedure have all been classified as depicting a landmark, the system may output or record the video image frame with the highest probability classification, in the middle of the landmark classified sequence of frames, etc. with an indication that the video image depicts the specified landmark.

With respect to the example intestine 1405, it may useful to recognize, e.g., a colonoscope image 1415b as depicting the left colic flexure, a colonoscope image 1415a as depicting the right colic flexure, a colonoscope image 1415e as depicting the ileocecal valve, and a colonoscope image 1415d as depicting the appendiceal orifice. These locations may serve as spatial “landmarks” 1410b, 1410a, 1410e, 1410d, respectively, since they represent recognizable structures common to the topology of the organ generally irrespective of the examination itself. In contrast to landmarks reflecting just structural features of an organ, some landmarks may also indicate operation specific events. For example, recognition of the rectum retroflexion landmark 1410c from image 1415c not only provides spatial information (i.e., where the endoscope's distal tip is located), but also indicates that the retroflexion operation is being performed. One will appreciate that landmarks may thus include both “normal” organ structures (the cecum, the terminal ileum, etc.) as well as “non-normal” structures (polyps, cancerous growths, tools, lacerations, ulcers, implanted devices, structures being subjected to a surgical manipulation, etc.).

One will also appreciate that context may allow one to infer additional information from the landmarks than just the spatial location of the examination device. For example, landmark recognition may itself provide temporal information when considered in view of the examination context due to the order of events involved. For example, in the timeline 1425, as time 1435 progresses, the computer system may recognize the indicated landmarks corresponding to times 1420a, 1420b, 1420c, 1420d, and 1420e (e.g., by timestamps for the time of acquisition of the corresponding visual image) between the procedure start 1430a and end 1430b times. Recognizing the “left colic flexure” at a second time 1420d after an initial recognition of the landmark at time 1420a may allow one to infer that the endoscope is in the process of being retracted. Such inferences may facilitate generation of a conditional set of rules for assessing the surgical examination. For example, if the right colic flexure landmark 1410a were recognized again after time 1420d, a rule set may indicate that the procedure is now atypical, as retraction would not typically result in the landmark 1410a again coming into view. Similarly, certain examinations and surgeries would not implicate certain landmarks and so those landmarks' appearance (e.g., the appearance of a suturing landmark during a basic inspection) may suggest an error or other unexpected action. As another example, the rectal retroflexion landmark 1410c is typically a final maneuver to complete an examination of the colon, and so landmarks recognized thereafter (or even an ongoing surgical recording beyond a threshold time following that landmark) may suggest anomalous behavior. For clarity, one will appreciate that landmarks may be identified in visual images corresponding both to fragments retained in the pose network and used to create a computer generated model, as well as those fragments which were removed. Thus, some embodiments may also include “smoke occlusion” or “biomass occlusion” as landmark events.

Landmark recognition may facilitate improved metadata documentation for future reviews of the procedure or examination, as well as provide real-time guidance to an operator or automated system during the surgical examination. Example systems and methods for providing such recognition are discussed in greater detail below. In some embodiments, video frames corresponding to recognized landmarks may be annotated, as, e.g., with label annotations 1440a indicating the landmark believed to be recognized, and probability annotations 1440b indicating the probability (or uncertainty) associated with the system's conclusion that the recognized landmark is indeed depicted in the image frame.

### Example Landmark Detection System Architecture

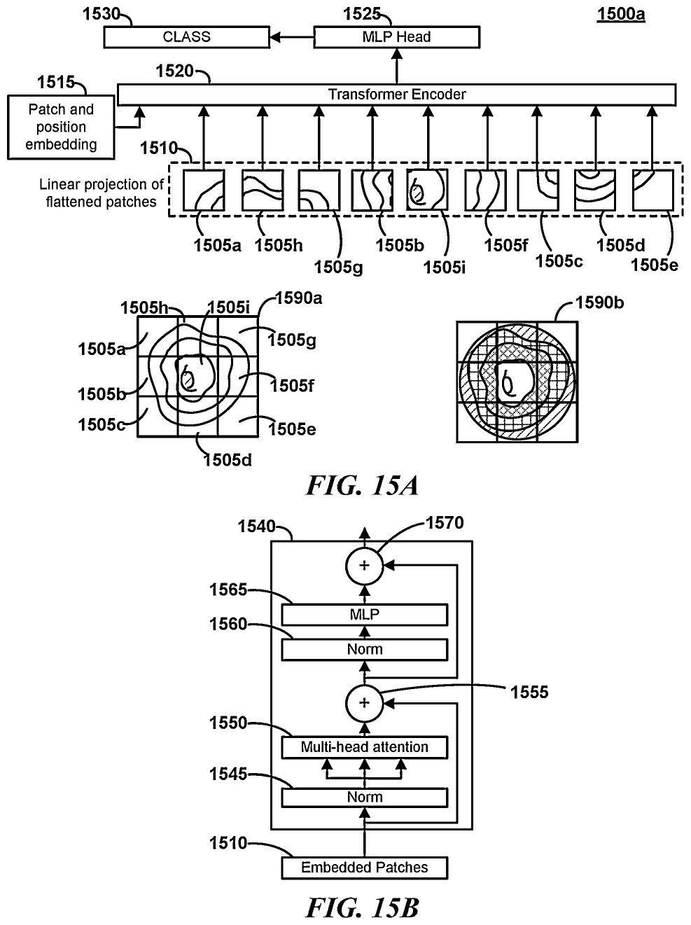

One will appreciate that many neural network architectures and training methods may suffice for recognizing landmarks from visual images or from depth frames (indeed, one may readily train a network to recognize landmarks from an input combining the two). For example, a sufficiently deep number of convolutional layers may suffice for correctly distinguishing a disparate enough set of desired landmark classes. As one example of a network found to be suitable for recognizing landmarks, FIG. 15A presents an example vision neural network processing pipeline 1500a for landmark detection, adapted from the paper “An image is worth 16×16 words: Transformers for image recognition at scale” appearing as an arXiv preprint arXiv: 2010.11929v2 and by Alexey Dosovitskiy, Lucas Beyer, Alexander Kolesnikov, Dirk Weissenborn, Xiaohua Zhai, Thomas Unterthiner, Mostafa Dehghani, Matthias Minderer, Georg Heigold, Sylvain Gelly, Jakob Uszkoreit, and Neil Houlsby. While the authors of that paper were repurposing transformer methods typically applied to Natural Language Processing (NLP) tasks for tasks in computer vision generally, a variation of their proposed architecture also produced viable landmark classification results in experimental instantiations of various of the disclosed embodiments. Such a network may provide a real-time “auto-tagging mechanism” for landmarks and may be trained to support multiple camera modalities. One will appreciate that the machine learning architecture may be implemented on-edge in the theater or remotely, e.g., on a server as in the cloud.

Specifically, given a visual image 1590a, the image may be divided into portions 1505a, 1505b, 1505c, 1505d, 1505e, 1505f, 1505g, 1505h, 1505i to form a collection 1510 of linearly projected linear patches for submission to a transformer encoder 1520 along with patch and position embedding information 1515. In some embodiments, position embedding information 1515 may be supplemented with the temporal state of the procedure (e.g., a time since a last recognized landmark). One will appreciate that such supplementation may occur at both training and at inference. A multi-layer perception head output 1525 may then produce predictions 1530 for each of the desired landmark classes. While visual image frames were used in the example implementation, depth frames, such as frame 1590b may be likewise processed, mutatis mutandis, for prediction in other embodiments.

As shown in FIG. 15B, the transformer encoder 1520 may employ several blocks 1540. For clarity, one will appreciate that the embedded patches 1510 may be normalized by block 1545 and provided to a multi-head attention layer 1550. Combined 1555 with the original inputs and normalized again at block 1560, the result may pass through an intermediate multilayer perceptron network 1565 before being added 1570 to the output from addition 1555 to form the final output for the block 1540. In various experiments, it was found that twelve such blocks 1540, applied in succession as part of transformer encoder 1520 produced acceptable landmark classification results for a colonoscopic examination procedure.

### Example Landmark Detection Deployment Pipeline

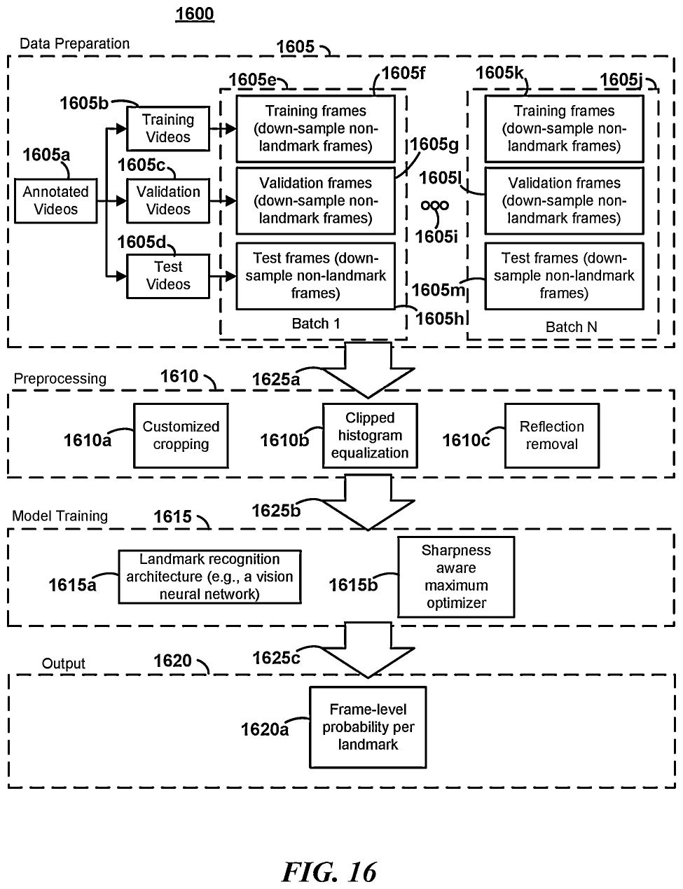

FIG. 16 is an example training pipeline 1600 for configuring and deploying a landmark recognition system, as may be implemented in some embodiments. During initial data preparation 1605 for training the system, videos 1605a from examination devices are annotated to identify frames corresponding to landmarks sought to be recognized. As mentioned, landmarks may be “normal” organ structures, as well as “non-normal” structures and temporal or surgical procedure specific events. These videos (or at least the annotated frames) may then be allocated into a training group 1605b, a validation group 1605c, and a test group 1605d.

Training successfully upon a video of one procedure may result in a model performing poorly upon a video of another procedure. Accordingly, training and testing may be separated by recording rather than by frame in some embodiments. Members from the groups 1605b, 1605c, 1605d may then be organized into batches. For example, a subset 1605f of the set of all frames appearing in the training videos 1605b may appear in batch 1605e, and a distinct subset 1605k in batch 1605j. In some embodiments, sets 1605f and 1605k may share common frames. Ellipsis 1605i indicates the presence of intervening batches. Corresponding sets may be selected for batches for the other video categories. Specifically, a set of frames 1605g may be chosen for batch 1605e as well as a set of frames 16051 may be chosen for batch 1605j (again, the sets may share some frames) from video 1605c. Finally, a set of frames 1605h may be chosen for batch 1605e as well as a set of frames 1605m may be chosen for batch 1605j (again, the sets may share some frames) from test videos 1605d.

One will appreciate that despite the linear presentation of this figure to facilitate understanding, various of the boxes, such as boxes 1605 and 1615, may direct one another's operations. For example, imbalanced training data may implicate certain compensating actions in some embodiments. That is, labeled image frame may be very rare in comparison to all the frames of a video and only a small number of videos may be available for the endoscopic context. Thus, rather than shuffle the data randomly, the data may be divided into those videos selected for training and those videos for testing and validation. This approach may help to build a much more generalizable model (e.g., compensating for the baseline disparities in camera intrinsics, in light sources, in patient anatomy, in operator behavior, etc.). Additionally, the data may be imbalanced, as there may be few frames of some landmark classes, such as retroflexion frames. Active learning may be used to compensate for these imbalances, performing subsequent training of the model upon frames with these landmarks specifically (especially the most difficult or edge cases) following general training upon the other data.

For reference, in some experiments there were 100 M frames available of other landmarks but only 5K were available for retroflexion. During training, the portion of the 5K frames upon which the system performed most poorly following general training were focused upon most heavily in the active learning phase so as to better ensure proportionate classification ability. Accordingly, during each epoch of active training, the training system or human training monitor may decide whether a class of frames should go into the training set or validation set depending upon how well the model performed upon the frames (e.g., placing more in training if performing poorly upon those classes, and more in validation if their classification is beginning to improve).

The batches of frames may then be provided 1625a to pre-processing operations 1610. Some embodiments apply some, none, or all, of the preprocessing operations 1610. For many videos, applying one or more of pre-processing operations 1610 may facilitate standardization among the frames in such a fashion as to improve machine learning training and recognition. Thus, one will appreciate that the choice of preprocessing operations 1610 during training may likewise be applied during inference (e.g., if customized cropping 1610a was applied to the image frames from the training batches, the same or equivalent preprocessing operations would be applied to image frames acquired during inference). One will further appreciate that the preprocessing operations 1610 may be applied in any suitable successive order (e.g., as one example, histogram equalization 1610b may follow customized cropping 1610a, which may itself follow reflection removal 1610c).

To facilitate the reader's understanding, a schematic representation of customized cropping 1610a is presented in FIG. 17A. Specifically, given an image frame 1705a (here shown depicting an inner cavity of an organ with a separate instrument within the field of view) it may be desirable for processing purposes to remove dark background border region 1705e (which naturally provides no information as to the contents of the field of view) as well as portions of the field of view outside a consistent region 1705b (e.g., a largest possible square fitting within the captured field of view). The processing operation 1705c may extract the portion of the image 1705a within the consistent region 1705b to produce the processed, cropped image 1705d.

FIG. 17E is a flow diagram illustrating various operations in an example process 1735 for performing such a cropping operation 1610a. Specifically, at block 1735a the system may convert the visual image 1705a from its original color space (e.g., RGB) to a color space with a brightness component, such as the YUV color space (which has a luminance component and two chroma components). At block 1735b, the system may then mask out the border (e.g., darkened region 1705e) using a threshold on the brightness component, e.g., the luma Y channel. At block 1735c, the system may then detect the center of the unmasked image and crop out a maximal rectangle corresponding to the consistent region 1705b. Naturally, one will appreciate that where fames are of differing dimensions, the process 1735 may also include scaling of the images 1705a or 1705b to a common width and height.

Similarly, to facilitate the reader's understanding, FIG. 17B depicts a schematic representation of clipped histogram equalization preprocessing operation 1610b. Here, the initial image 1710a (which may be the post-cropping image 1705d) depicts a foreground region 1710c of noticeably greater luminance than a darker region 1710d. Applying histogram equalization operations 1710e may produce an image 1710b wherein the darker region 1710b and region 1710a now share a more similar luminance.

FIG. 17F is a flow diagram illustrating various operations in an example process 1740 for performing a clipped histogram equalization operation as depicted in FIG. 17B. At block 1740a, the system may convert the image from its original color space to a color space with luminance, such as YUV (if this was not done already, e.g., as part of the operations of process 1735). At block 1740b, the system may apply an adaptive histogram equalization algorithm, such as the Contrast Limited Adaptive Histogram Equalization (CLAHE) to the luminance channel (e.g., the Y channel) of the image. If desired, the image may be converted back to its original color space (or the suitable color space for machine learning processing) at block 1740c (though one will appreciate that such a conversion may occur following the last of the preprocessing operations).

Finally, FIGS. 17C and 17D depict aspects of the reflection removal preprocessing operation 1610c. Specifically, just as occurs in many strongly lit point light source photography environments, FIG. 17C demonstrates how light from the light source 1720c may travel from a flat 1720d surface or curved surface 1720e so as to enter camera 1720b of the visualization tool 1720a so as to create a highlight (either through paths 1725a, 1725b or paths 1725c, 1725d, respectively). Within many organs, fluids and textures may often cause surfaces to reflect light from the light source 1720c in this manner so as to create a bright highlight. Naturally, it is unlikely that two different encounters with the same landmark with two different examination devices in two different patients (and even with in the same patient or with the same device) will precipitate the exact same highlight distribution, particularly given the non-rigid character of many organs. Consequently, it may be imprudent to include highlights in the training and inference processes as this may result in overfitting of the resulting model to unique highlighting patterns in the training images. Thus, as shown in FIG. 17D, given an image 1730a wherein specular highlights 1730d are manifest upon tissue surfaces and contours, processing 1730c may produce an image 1730b with such highlights removed. Again, for clarity, one will appreciate that the image 1730a may be one of images 1705d or 1710b.

FIG. 17G is a flow diagram illustrating various operations in an example process 1745 for performing such a highlight removal. At block 1745a, the system may convert the image from its original color space to a color space with a brightness component, such as YUV (if this was not done already, e.g., as part of the operations of process 1735). At block 1745b, the system may detect highlights as those pixels exceeding a threshold, e.g., those pixels in the 99th percentile in brightness across all pixels in the batch, across all the training images, etc. Alternatively, outliers in absolute brightness may also be removed (e.g., above a standard numerical threshold, such as a luminosity above 250 when constrained to a range of 0 to 255). At block 1745c, the system may use an in-paint algorithm, such as OpenCV™'s Telea algorithm to replace the pixels identified at block 1745b as belonging to highlights, with appropriate values (e.g., surrounding non-highlight texture values). One will appreciate a variety of suitable algorithms for this purpose, including manual methods, such as application of the cloning tool in conjunction with surrounding non-highlighted pixels in many popular graphic editing software packages (though, naturally, such a manual method would be inefficient for real-time inference during surgery).

Returning to FIG. 16, following the zero or more preprocessing operations 1610 (again, in any suitably preferred order) the system may proceed 1625b to the model training operations 1615 using the batches to produce a vision neural network 1615a (e.g., as discussed with respect to FIG. 9A) and (in some embodiments) to utilize a sharpness aware maximum optimizer 1615b (e.g., as described in the paper “Sharpness-Aware Minimization for Efficiently Improving Generalization” appearing as arXiv preprint arXiv: 2010.01412v3 by Pierre Foret, Ariel Kleiner, Hossein Mobahi, and Behnam Neyshabur). These tools may then be used at inference (e.g., on-site in the surgical theater, in the cloud as part of a server computer system, etc.) to produce 1625c output 1620 such as a probability of a landmark appearing in a visual image frame 1620a. One will appreciate that from a corpus of possible landmarks, the trained system receiving a frame may produce a probability for each landmark class. In some embodiments, the landmark class associated with the highest prediction probability may be taken as the predicted landmark. If no probability exceeds a threshold, then the image may be classified as not depicting any landmark (though some embodiments may include training and inference which include “non-landmark” classes).

### Example Graphical User Interface

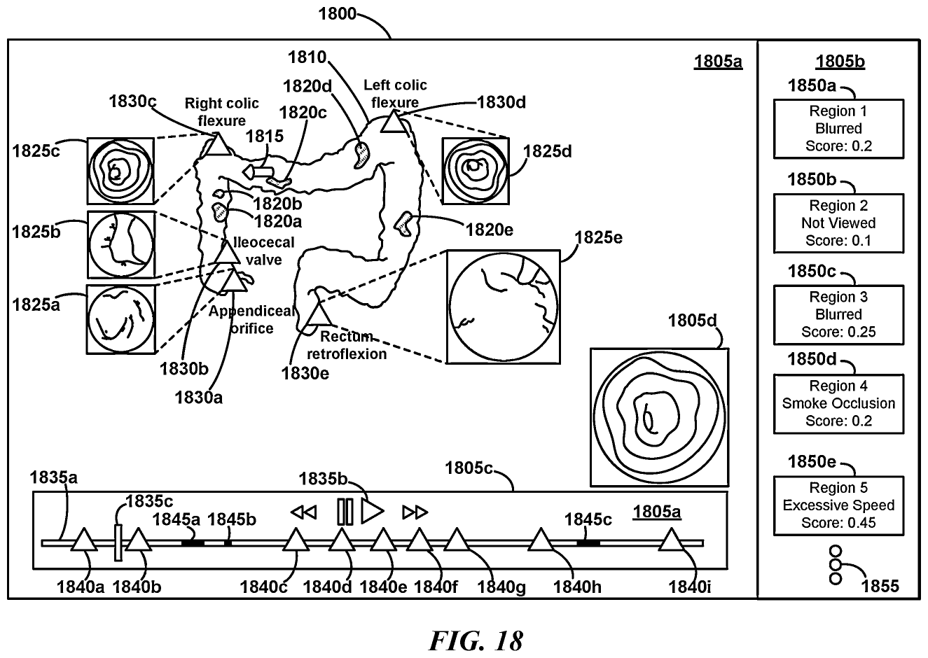

FIG. 18 is a schematic representation of an example graphical user interface (GUI) 1800 for internal body structure data review, as may be implemented in some embodiments. In this example, the interface 1800 may generally include four regions: a computer generated model viewing region 1805a, an artifact listing region 1805b, a playback control region 1805c, and a video playback region 1805d. One will appreciate that not all of the regions need be present, nor exactly as depicted here, and that additional regions may be present.

Computer generated model viewing region 1805a may provide the user with a view (possibly translatable, rotatable, zoomable, etc.) of the computer generated model of the body interior structure (e.g., a large intestine computer model 1810 as shown) generated, e.g., using various of the methods disclosed herein. In some embodiments, the model 1810 may simply be a generic model, rather than a model generated from surgical data, and used as a reference upon which to overlay landmarks, data from the surgier, portions of the data-generated model, etc. As the procedure playback proceeds, a current timepoint indicia 1835c may advance along a time indicia 1835a, depicting the time in the total playback at which a frame currently presented in the video playback region 1805d was generated. An icon, such as the three-dimensional fiducial 1815 (here, a three-dimensional arrow mesh) may then advance through the model of the structure (whether the model was generated from surgical data or the model is a generic model with the nearest position inferred), in the orientation and position most resembling the position and orientation corresponding to the current playback position (e.g., the orientation of the camera 210a upon the distal tip 205c). One will appreciate that the three-dimensional fiducial 1815 may correspond with the current playback position 1835c throughout the playback. For example, when the fiducial 1815 is an arrow, or other structure indicating the orientation of the capturing sensor, then the visual image depicted in region 1805d may provide the viewer with an intuitive understanding of the state of data capture, state of the procedure, etc.

In the depicted example, the model 1810 is a model generated from surgical data using the methods and apparatuses described herein. Consequently, various regions of the model have been marked (e.g., via a change in texture, vertex coloring, overlaid billboard outline, etc.) as being associated with three-dimensional artifact indicia 1820a, 1820b, 1820c, 1820d, 1820e. These artifacts 1820a, 1820b, 1820c, 1820d, 1820e may correspond to portions of the playback where smoke appeared in the frame, an occlusion occurred, the operator's motion precipitated a blurred image, holes in the model were identified, etc. Accordingly, artifact listing region 1805b may include representations 1850a, 1850b, 1850c, 1850d, 1850e corresponding to some or all of the artifacts 1820a, 1820b, 1820c, 1820d, 1820e. For example, the representation 1850a indicates that a first region (e.g. associated with artifact 1820a) was blurred. As indicated by ellipsis 1855, there may be more representations than shown (e.g., where there are many artifacts, then only subsets of indicia may be presented at a given playback time in the GUI 1800). In some embodiments, the representations may include a quality score. Such a score may indicate why such a region was marked as corresponding to an artifact or may indicate a quality of the operator's review for the region (e.g., a coverage score as described herein). Naturally, some artifacts may represent multiple defects. For example, representation 1850b indicates that a region was not viewed (e.g., corresponding to artifact 1820b). This is both a failure on the operator's or automated system's part to examine a given region visually, but also a defect in the resulting model, as the model generation process lacked adequate information about the region to complete the model 1810 (one will appreciate that such indicia could also be used to indicate a corresponding position in a generic model, e.g., by determining similar vertex positions in the respective meshes, or in a model wherein the holes have been supplemented as in supplemented model 1110b).

In addition to artifacts, some embodiments may simultaneously or alternatively present landmark indications 1830a, 1830b, 1830c, 1830d, 1830e which may be associated with corresponding indications of the frame or frames 1825a, 1825b, 1825c, 1825d, 1825e in which the respective landmark was recognized (again, one will appreciate that the depiction here is merely exemplary and the frames may be presented instead as, e.g., overlays in playback control region 1805c, highlighted as they appear in video playback region 1805d, both, not presented at all, etc.). As indicated, text indications (such as the name of the landmark) and other metadata (e.g., the classification probability or uncertainty) may also be presented in the GUI for each landmark. Such supplemental data may be useful, e.g., where a reviewer is verifying the billing codes used for the procedure. For example, in some embodiments, certain landmarks may be associated with reimbursement codes, which are indicated in the metadata, allowing a reviewer to quickly confirm a procedure's proper coding after a surgery by reviewing the GUI interface.

The reviewer may use controls 1835b to control the playback of the operation. One will appreciate that artifacts 1820a, 1820b, 1820c, 1820d, 1820e and the landmark indications 1830a, 1830b, 1830c, 1830d, 1830e may be displayed a-temporally (e.g., as shown here, where all are presented simultaneously), temporally, wherein they are as shown or highlighted as their corresponding position in the playback occurs, etc. Temporal context, such as this, may help the reviewer to distinguish, e.g., encounters with the same landmark at different times, when during the procedure the landmark occurred or was encountered, etc. Thus, in some embodiments, the timeline 1835a in playback control region 1805c may include both temporal landmark indicia 1840a, 1840b, 1840c, 1840d, 1840e, 1840f, 1840g, 1840h, 1840i and temporal artifact indicia 1845a, 1845b, 1845c. Thus, e.g., the occurrence of blur represented by representation 1850a may also be represented by temporal artifact indicia 1845a and by three-dimensional artifact indicia 1820a. Clicking on a temporal landmark indicia or temporal artifact indicia may advance playback to the first frame corresponding to the detection of the landmark or occurrence of the artifact. For holes, clicking the artifact indicia may advance playback to a first frame adjacent to the hole (subsequent clicks advancing to other of the acquired frames adjacent to the hole).

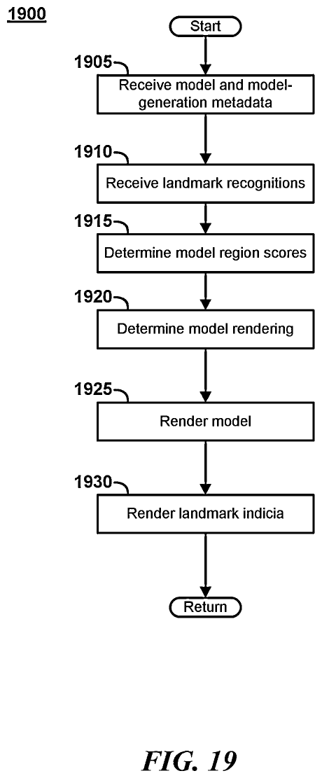

FIG. 19 is a flow diagram illustrating various operations in a process 1900 for presenting internal body structure review data as may be implemented in some embodiments. At block 1905, a computer system may receive a computer generated model and model metadata. For example, the computer generated model may be the mesh generated in accordance with various of the methods described herein, and the metadata may be identified holes, artifacts, etc. Similarly, at block 1910, the system may receive indications of the recognized landmarks (e.g., the landmark type, original data corresponding to the landmark, etc.).

At block 1915, the system may determine scores associated with the metadata or the model as a whole. For example, in some embodiments, the model may be divided into distinct sets of vertices (corresponding, e.g., to various fragments) and each set associated with a quality score over time. Initially, as most of the model is not viewed, most sets will receive poor scores. As the regions associated with the sets are more adequately reviewed, they may, generally, receive higher scores. However, sets associated with regions that have holes, were occluded by smoke, poorly reviewed, etc. may receive a lower score. Absent a revisit to the region to correct any such deficiency, the set's final score may remain low.

At block 1920, the system may determine the model's rendering. For example, the system may choose vertex colorings in accordance with identified holes (e.g., providing distinguishing color or texture renderings for in-filled regions 1130a, 1130b, 1130c, 1130d), determined scores, artifacts, etc. The model may then be rendered at block 1925, e.g., in region 1805a. Similarly, at block 1930, various landmark indications may be presented in the GUI, e.g., temporal landmark indicia 1840a, 1840b, 1840c, 1840d, 1840e, 1840f, 1840g, 1840h, 1840i or landmark indications 1830a, 1830b, 1830c, 1830d, 1830e.

### Example Review Scoring Method

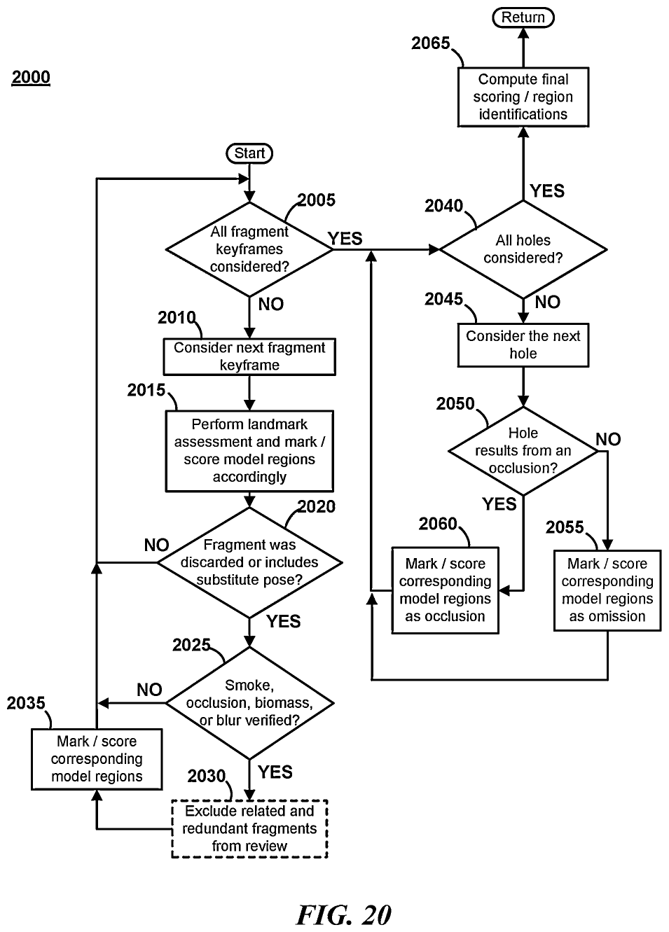

FIG. 20 is a flow diagram illustrating various operations in a process 2000 for scoring, or marking, internal body structure review data, e.g., for adjusting the model rendering, as may be implemented in some embodiments. For example, during or following generation of all or a portion a model of an internal body structure using the process 500 or pipeline 600, a computer system may seek to identify portions of the surgical procedure (e.g., at block 1915) during which adverse events occurred or which were subject to undesirable degrees of review. Some landmarks (e.g., a perfusion) may themselves be suggestive of events that adversely or positively impacted the procedure, and should accordingly result in a scoring or GUI adjustment. Scoring may facilitate real-time or offline evaluation of a patient, of the examination, and of the operator. Thus, a single score may serve multiple purposes, as when it is used to help an operator appreciate the comprehensiveness of their review during a surgery, to indicate operator improvement over multiple surgeries (granular scores indicating how the operator may improve, e.g., by avoiding movements precipitating blur, avoiding departures from the centerline, etc.), to indicate a likelihood of a patient possessing overlooked complications, etc. Low or poor scores may suggest, e.g., that the procedure was insufficient to exclude cancer in the colon, whereas high or good scores may indicate that there is low probability of the colon containing cancer based upon the procedure.

As discussed below, scoring algorithms may consider detection of uncovered regions in the colon, withdrawal duration of the sensor device, internal body structure “cleanness” evaluation (e.g., based upon a lack of occlusions or subsequent data captures serving to compensate for regions occluded by biomasses). A “withdrawal duration” used for scoring determinations may be assessed, e.g., for colonoscopy, as the time from the last detected cecum frame (e.g., the last frame classified as a cecum landmark) until the camera left the patient body minus the time it took to remove polyps identified in the patient. Thus, a score may be determined from withdrawal duration, landmark (cecum) detection, and polyp removal times (e.g., a ratio of the withdrawal duration to the sum of the removal times). The system may aggregate these scoring results into a human readable format accessible to the operator and other reviewers. Thus, the withdrawal duration is the duration of the scanning process alone, without the time it took to reach the cecum or the time of actions in the procedure, such as removal of polyp. Out of body frames may be detected, e.g., based upon luminosity alone, as well as using a trained machine learning architecture. Some embodiments may determine a “withdrawal time per colon segment”, by allocating the overall duration to the corresponding segments of the internal body structure. Such an approach may facilitate more granular assessments, e.g., “time in ascending colon”, “time in transverse colon”, etc. Known metrics may also be used in some embodiments, such as the Boston Bowel Preparation Score (BBPS). As discussed, a coverage score may be determined for segments of the model or the model as a whole. The system may aggregate these varying types of scores into a human readable format accessible to the operator and other reviewers.

In the depicted example process for scoring and marking, the system may iterate through fragments at blocks 2005 and 2010. This iteration may occur over, e.g., a most recent set of fragments generated while the procedure is ongoing or the totality of fragments generated after the procedure has finished. In the depicted example, each fragment's keyframe may then be generally subjected to a landmark identification process at block 2015 (e.g., upon the keyframe's depth frame or upon the corresponding visual image) and any adverse event detected at block 2020.

In this example, landmark detection may occur prior to consideration of the fragment's pose or removal as instruments, temporary deformations of the body interior, etc., may all be symptomatic of a landmark, but sufficiently atypical from the normal contour of the body's interior that the fragment could not be integrated into the complete model. However, one will readily appreciate variations to this approach, as when, e.g., block 2020 performs a filtering function, so that landmark identification is attempted only upon “clean” images following a NO identification at block 2020 (in some embodiments, the system may verify that a cleaning action landmark, e.g., with irrigation, has occurred following an unclean frame assessment). Naturally, such filtering may occur independently of any scoring operation so as to improve landmark detection and identification.

Returning to the depicted example, at block 2020, the system may determine if the fragment, based upon its keyframe, has been identified, explicitly or implicitly, as defective. For example, where the model is complete and a final global pose estimation and integration performed, the isolation of a fragment from the network used to generate the completed model (as in the case of fragment 925b) may suggest either an occlusion (as when the endoscope collided with a wall or other structure, as in the situation at time 910b), smoke, biomass (feces, blood, urine, sputum, etc.), or other adverse event blocked the field of view. Often distinctions between such events may be made based upon the corresponding visual image (by texture, luminosity, etc.) or depth values (e.g., near, very planar depth values). One can readily train a machine learning architecture upon one or both of these datasets to classify between the possible events.

Where an adverse event has been detected at block 2025, the system may mark the corresponding portion of the model or adjust a score accordingly at block 2035. For example, the occurrence of the event may be noted in the timeline and its relevance to the surgical review (e.g., its distracting potential) recorded. Smoke, for example, may be an undesirable event for a given surgical task and so the event's presence may result in a decrease in the overall score for the review at that point in time. Biomass may result in degradation of surgical instrument effectiveness, or be indicative of the state of the patient, and so its detection may likewise result in a score adjustment (e.g., bleeding should not occur in a standard bowel examination). Similarly, one will appreciate that the model vertex colorings or textures may likewise be animated or adjusted throughout playback. Accordingly, the occurrence of an event may be denoted by, e.g., a change in vertex coloring at the appropriate portion of the model (naturally, where the fragment under consideration was discarded from the model's generation, one may, e.g., use the temporally neighboring fragment that was included, in combination with the discarded fragment's timestamps, to identify the spatiotemporal location).

While the complete removal of a fragment (or group of fragments) in some embodiments may suffice for detecting adverse events at block 2020, in some embodiments, less than complete removal may also suffice to invite examination at block 2025, e.g., resulting from simple substitution of poses, as discussed at block 575. For example, the neural networks 715a and 715b may be trained upon a wide variety of data, some of which may deliberately include adverse events and occlusions, and so may consequently be more robust to pose generation, facilitating a sufficiently correct pose and depth determination that the fragment is not removed at integration 680. Thus, in some embodiments, detection of a successful pose substitution at block 2020 may also result in a YES transition to block 2025.

At block 2025 the system may verify the presence of smoke based upon an analysis of the hue, texture, Fourier components, etc. of the visual image corresponding to the depth frame. Similarly, blur may be confirmed at block 2025, by examining the Fourier component of the visual image (a lack of high frequency detail may suggest blurring due to motion, liquid upon the lens, etc. with optical flow possibly being used to distinguish the types of blur). One will appreciate similar methods for assessing the visual or depth image, including application of a neural network machine learning architecture. In some embodiments, real-time feedback may be provided to the operator, e.g., via a “speedometer” type interface, indicating whether the distal tip of the sensor device is traveling too quickly. As indicated by block 2030, fragments related to the fragment under consideration may be excluded from review in the iterations of block 2005 if they will not provide more information (e.g., all the fragments are associated with the same smoke occlusion event).

Following the iterations of block 2005, the system may then consider any holes identified in the model at block 2040. Where the entire model has been generated and the procedure is complete, holes identified throughout the entire model may be considered. However, where the process is being applied during the procedure, one will appreciate that the hole identification system may only be applied at a distance from the current sensor position so as to avoid the identification of regions still under active review as possessing holes (alternatively, the identification system can be applied to all of the partially generated model and those holes near the current distal tip 205c position simply excluded).

In either event, for each of the holes to be considered at block 2045, in the depicted embodiment the system may seek to determine whether the hole results from an occlusion or merely from the system or operator's failure to visit the desired portion of the anatomy. In other embodiments, all holes may be treated the same, as simply omissions to be identified and negatively scored in proportion to their surface area. In the depicted example, distinguishing between occlusions and regions simply not viewed facilitates different markings or scorings at block 2055 and 2060. For example, some interior body structures may include a number of branchings, such as arteries, bronchial tubes, etc. Electing not to travel down a branch may have a considerably different effect upon scoring than simply failing to capture a region occluded by a fold, ridge, etc. Thus, at block 2050, the system may consider fields of view of various fragments' global poses, or omitted fragments, and determine if the hole was an omission precipitated by an occluding fold, etc., or was a region beyond the range of the depth capturing abilities. Both situations may result in holes in the model, but the operator's response to each during or after a procedure may be considerably different, as some occlusions may be expected while others may reflect egregious omissions, and similarly, some untraveled routes may be anticipated, while other untraveled routes may reflect a mistake.

Once the sub-scores for various adverse events (e.g., at block 2035) and holes (e.g., at blocks 2060 and 2055) have been determined, the system may determine a final score and model presentation at block 2065 based upon the constituent results. The final score may be determined as a weighted sum of one or more of the scores from blocks 2035, 2060, 2055, the current, or overall, coverage score, the withdrawal duration score, cleanness scores (e.g., as determined at block 2055 or block 2035), a BBPS score, etc. The final score may be presented as part of GUI 1810 (e.g., updated as playback proceeds), during the procedure (to advise the operator or automated system on its current performance), or after the procedure, e.g., as part of a consolidation among procedures (e.g., to track an operator's progress over time and to provide feedback). One will appreciate that all or some of the operations discussed herein may be applied on a rolling basis during the surgery upon previously captured portions of the model, providing online guidance and guiding the physician to go forward, backwards, look left, right, to improve coverage of the internal body structure.

### Data Processing for GUI Presentation Overview

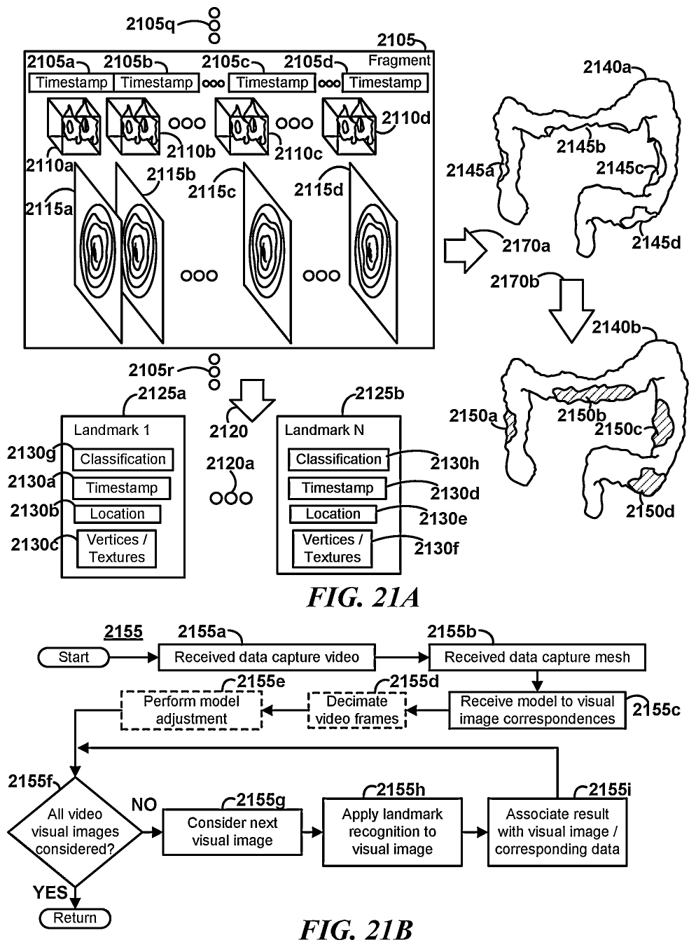

To provide context and to facilitate the reader's understanding, FIG. 21A is a schematic representation of inputs to a Graphical User Interface (GUI) data management system as may occur in some embodiments. Specifically, various of the disclosed embodiments may include a computer system configured to receive examination data and to process the data into a form amenable to intuitive presentation in a GUI. The system may receive the raw visual input data as described above and apply various of the disclosed methods to generate fragments, such as fragment 2105 (additional fragments being presented by ellipses 2105q and 2105r), and to assemble 2170a the fragments so as to form resulting model 2140a. Similarly, the hole in-fill procedures disclosed herein may be applied 2170b to replace or identify holes 2145a, 2145b, 2145c, and 2145d, with supplemented regions 2150a, 2150b, 2150c, and 2150d, respectively. Finally, the system may apply the landmark identification method disclosed herein to identify landmarks 2125a, 2125b (intervening ellipsis 2120a indicating the possible presence of other landmarks).

One will appreciate that in some embodiments, rather than receiving the raw visual image video data, the system may instead receive all or some of the completed fragments, models, and landmark materials. For example, the system may receive only the fragments and be able to generate model 2140b and landmarks 2125a, 2125b on its own. As the fragments, such as fragment 2105, may be structured to include visual image frames 2115a, 2115b, 2115c, 2115d, depth frames 2110a, 2110b, 2110c, 2110d, and corresponding timestamps 2105a, 2105b, 2105c, 2105d, it may be possible to infer various metadata for the models 2140a, 2140b and landmarks 2125a, 2125b. For example, the first collection of data sets in each fragment, e.g., the set formed of visual image frame 2115a, depth frame 2110a, and timestamp 2105a, may serve as a keyframe, as described elsewhere herein, for creation of model 2140a. Because the fragments include visual data, e.g., visual image frame 2115a, the system may apply the landmark recognition system to these images to generate 2120 the landmarks. Thus, the computer system may use the relation between the visual image 2115a, depth frame 2110a, and timestamp 2105a data to likewise identify correspondences between the playback (e.g., based upon timestamp 2105a), generated landmarks 2125a, 2125a (e.g., based upon visual image 2115a), and models 2140a, 2140b (e.g., based upon the global pose estimation integration of the fragment 2105 into the model). As described in greater detail herein, these correspondences may facilitate integrated presentations of the data to the user as well as facilitate coordinated interactions by the user with the different types of data.

One will appreciate that the “timestamps” in this diagram may refer to actual points in time, or may simply be indicators of the relative placement of each data capture. Thus, a timestamp may simply be the ordering number of the data capture in some embodiments (e.g., if fragment 2105 were the first captured fragment, then timestamp 2105a may be 1, timestamp 2105b may be 2, timestamp 2105c may be 3, etc.). The ordering of fragments and their constituent data frames based upon the timestamps may likewise facilitate the creation of temporal metadata in the corresponding portion of the model 2140a and corresponding landmark (e.g., landmark 2125a).

The data structure used to represent landmarks 2125a, 2125b may include a record of the identified landmark classification, e.g., classifications 2130g, 2130h, and the timestamp 2130a, 2130d corresponding to the timestamp of the set in which the visual image frame from which the landmark was identified (e.g., one of timestamps 2105a, 2105b, 2105c, 2105d). Following creation of model 2140a or model 2140b, the landmark data may also be updated to reflect the location 2130b, 2130e (e.g., the model vertices, an averaged point, a convex hull enclosing the relevant vertices, etc.) in the model 2140a or 2140b in which the data corresponding to the visual image was integrated. As mentioned, in some embodiments, some landmarks may be identified in visual images of fragments which were ultimately excluded from the model and may consequently correspond to one of holes 2145a, 2145b, 2145c, 2145d. Thus, in these landmarks' data structures the location 2130b may be inferred from the location in the model of temporally neighboring fragments, which were included in the model (e.g., to identify corresponding holes). Thus, in some embodiments the location for such fragments may be identified as a hole (e.g., hole 2145a) or in-filled region (e.g., region 2150a). Including the recognized landmarks for these images in the results may be useful for diagnosing the reason for poorly acquired coverage and for the presence of holes in the model. Indeed, in some embodiments, the landmarks may be a set consisting entirely of “adverse” events (smoke, biomass occlusion, collisions, etc.) and only excluded fragments' visual images may be examined for a landmark's presence. This may allow the reviewer to quickly understand where, and why, portions of the examination were defective.

In some embodiments the vertices and textures 2130c or 2130f in the model 2140a or 2140b may also be associated with a landmark. One will appreciate that though this metadata is shown in the same box 2125a, 2125b for each landmark, in implementation these data values may be stored separately and simply referenced (e.g., by pointers to data locations), e.g., by a central index. Again, by cross-referencing depth data, visual data, landmark data, hole data, and model data, the system may present an integrated representation of the surgical data to the reviewer as described herein.

To accomplish such cross-referencing, a processing protocol, such as that shown in FIG. 21B may be applied to the incoming data. In this example process 2155, the mesh (either model 2140a or 2140b) has already been prepared, but the landmarks have not yet been identified. Accordingly, the computer system may receive the original video capture at block 2155a and mesh at block 2155b. At block 2155c the system may receive the model to visual image correspondences. For example, the fragments may indicate which portions of the model were generated using depth frames corresponding to certain of the visual image frames, as shown in FIG. 21A. One will readily appreciate that many systems may identify the correspondences in many other ways, e.g.: with an associative array relating portions of the model to the visual images, with an index of timestamps mapping locations in the model (at which depth frames were integrated) with corresponding visual images, etc. The correspondence references may also include identifications of the lack of correspondence (i.e., that the visual image is not associated with any portion of model 2140a), as when fragments were rejected from the global pose network used to generate model 2140a.

As the visual image capture rate of the camera may be quite high, there may be redundant information in the video frames. Accordingly, in some embodiments, only frames at intervals may be considered for landmark identification (as a sequence of substantially identical frames would likely simply result in the same classification result). Thus, at block 2155d the computer system may decimate or otherwise exclude certain of the visual images from landmark processing. Similarly, in some embodiments, at block 2155e, the system may perform any model adjustments, such as hole identification and supplementation, to create model 2140b. Block 2155e may likewise include the association of some landmarks with holes or in-filled regions. At blocks 2155f and 2155g, the system may then iterate over the visual images to attempt to detect landmarks at block 2155h. The classification results may then be associated with the visual image at block 2155i, which, as discussed above with refence to FIG. 21A, may imply a reference to a corresponding portion of the model 2140a, 2140b (including a hole 2145b or supplemented region 2150b), related timestamps in the playback, etc.

Pre-processing, as in this example, may facilitate a more comprehensive presentation of the examination to the reviewer as well as a more comprehensive determination of the examination's quality (as discussed herein, such processing may occur in real-time upon previously reviewed regions to guide operator follow-up coverage). For example, having a supplemented model 2140b that strives for real-world fidelity may improve quality assessment of the review (as when the surface area of model 2140a is compared with that of model 2140b to determine how egregious the size of the holes in the review were, based upon the difference or ratio in surface area or volume). Further supplementing the model with landmark identification may also serve to anchor the review to recognizable locations and events. For example, a landmark may indicate, e.g., that a hole corresponds to a gastric bypass or other operation changing the volume or structure of the internal body structure. This may be important knowledge as it may allow the system to adjust the supplemented region 2150b, either discounting the difference in surface area from the model 2145b or otherwise acknowledging that the supplemental region 2150b is atypical. Similarly, recognizing landmarks in covered regions of the model 2140a may facilitate spatiotemporal orientation of the reviewer and confirmation of other automated processes. For example, a simple preventative care examination may have taken three times as long as expected and so may initially receive a below average score. However, recognition of a landmark corresponding to a polyp or cancerous growth early in the procedure may alert the system or reviewer to the atypical character of the review and that the operator's extended response time was not therefore unusual.

Combining landmark detection with model generation may also help ensure the fidelity of each set of data. For example, the system may verify that a landmark prediction agrees with its corresponding location in either model 2140a or 2140b, such as that recognition of the right colic flexure corresponds to a location in the proper corresponding quadrant of the model. Conversely, the system may also confirm that landmarks identified as not having any correspondence with the model, such as those associated with fragments which were excluded from the graph pose network, include classification results consonant with such exclusion, e.g. a landmark prediction of smoke or biomass occlusion. For example, in the model 2140a, multiple landmarks may be identified in connection with visual images whose fragments were not used in creating the model. Examination of neighboring fragments may indicate, however, that the omitted fragments are all associated with hole 2145b. By supplementing the model with supplemental region 2150b, the system or reviewer, may more readily identify where in the supplemental region 2150b the landmark was likely to have occurred (e.g., if cauterization was only applied at a specific location of the supplemental region 2150b, the location may be more readily identifiable with the supplemental region 2150b visible in the model 2140b, than when it is absent in the model 2140a). While discussed here in connection with holes missing in the model so as to facilitate understanding, one will appreciate that the disclosed correspondence methods may likewise be applied in models without holes (where, e.g., in a subsequent pass the endoscope captured fragments of the missing region, thereby completing the missing region, but the earlier landmark occurrence, such as smoke, precipitating the hole is still found to correspond with the region, albeit, at an earlier timestamp than the timestamp of the subsequent pass completing the missing region).

### Example Mesh Alignment

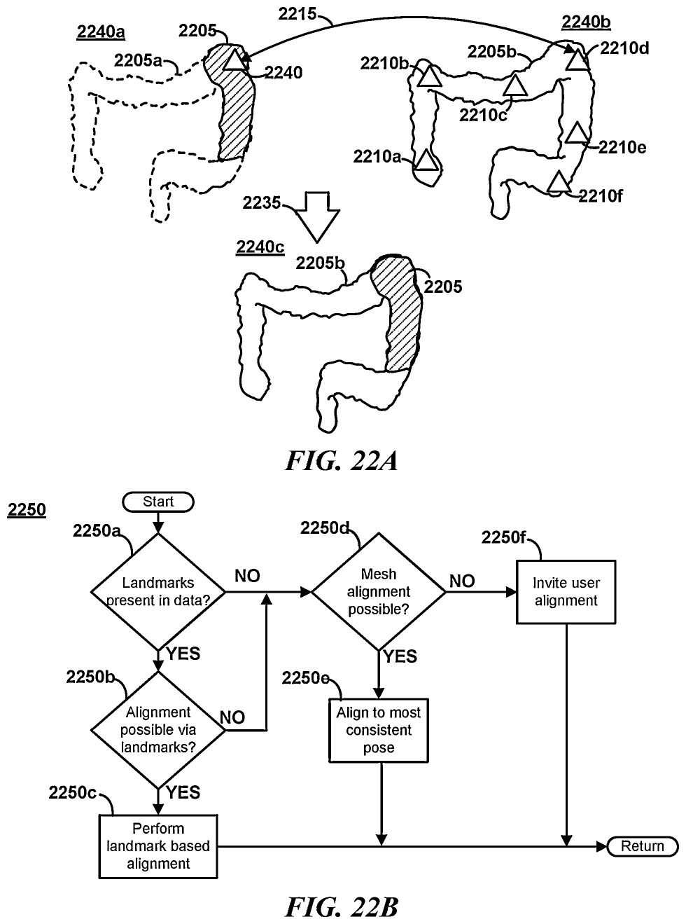

As another example of the benefits of identifying correspondences between landmarks and mesh structures, FIG. 22A provides a schematic representation of an example mesh alignment operation as may be performed in some embodiments. Specifically, one will appreciate that many procedures may provide data on only a portion, perhaps a very small portion, of an internal body structure. This may be natural in minimally invasive procedures, where only a portion of the organ or tissue is to be examined. Even where the procedure involves traversing a significant portion of the internal body structure, operators may not activate the data capture system until specific portions of the surgery, again resulting in limited mesh reconstructions.

Accordingly, given a mesh 2205 depicting only a portion of an internal body structure 2205a (one will appreciate that the dashed portion is provided here merely to facilitate the reader's understanding and that the mesh does not include any of the dashed region 2205a) as shown in state 2240a, the system may seek to align the mesh 2205 relative to an “idealized” virtual representation of the body structure 2205b (e.g., an average of meshes across a representative patient population, an artist's rendition of the internal body structure, etc.). Such alignment may facilitate placement of the mesh 2205 relative to the idealized virtual representation or texturing of a portion of the idealized virtual representation mesh 2205b in a GUI (e.g., using visual images from the examination), thereby providing the reviewer with an intuitive understanding of the relative position of the captured data relative to the internal body structure in question. Indeed, in some embodiments, the idealized virtual representation may be of the entire, or of a significant portion of, a patient's body, thereby allowing the reviewer to quickly orient their review to the region of the body in question.

The idealized virtual model 2205b, as shown in ideal state 2240b, may already include a plurality of landmark markers 2210a, 2210b, 2210c, 2210d, 2210e, 2210f (e.g., corresponding to known identifying regions of the body structure, such as the right colic flexure, ileocecal valve, left colic flexure, appendix, etc.). Each of these markers may indicate a location in the idealized model 2205b where then landmark is typically found in most patients. The markers may also include conditional rules, e.g., that certain landmarks may only be encountered following the identification of other landmarks (e.g., where landmarks are associated with successive tasks in a surgical procedure).

In some embodiments, it may readily be possible for the computer system to align 2235 the mesh 2205 with the idealized mesh 2205b as shown in state 2240c based upon their vertex structures. For example, many known pose alignment algorithms employing, e.g., simulated annealing or particle filtering methods, may be used to identify a best fit alignment of mesh 2205 with the idealized mesh 2205b. However, as patient anatomy may vary considerably and as such alignment methods may be computationally intensive, various of the embodiments seek to employ recognized landmarks (e.g., landmark 2240) associated with the visual images of mesh 2205 so that the correspondence 2215 with landmarks (e.g., landmark 2210d) may be used to more quickly align the meshes (thereby more readily facilitating, e.g., real-time alignment). For example, identifying such a correspondence 2215 may serve as an “anchor” by which to limit the search space imposed upon the search algorithm. Rather than a search space including any possible translation, rotation, and scaling of model 2205, now the system need only find the most appropriate rotation and scaling about the location associated with landmark 2210d.

To further facilitate the reader's understanding, FIG. 22B illustrates an example process 2250 for performing such an alignment. Specifically, after having attempted to find landmarks in the provided examination data (e.g., at block 2155h), the process 2250 may now consider if any landmarks were identified at block 2250a. Naturally, landmarks identified in visual images corresponding to depth frames that were used in creating mesh 2205 may be used for alignment. However, as previously discussed, even landmarks identified in visual images corresponding to depth frames which were not integrated may also improve alignment as, e.g., when a centroid of a hole having a recognized landmark is used to reduce the alignment search space.

Where no landmarks suitable for alignment were found at block 2250a, the system may consider if mesh exclusive alignment is possible at block 2250d (e.g., if the process is not being run in real-time or if computational resources exist for performing a best fit alignment) the alignment may be performed at block 2250e. Where such process fails, or constraints do not facilitate its being run, then at block 2250f the system may invite the user to perform the alignment manually (e.g., using a mouse to translate, scale, and rotate the captured mesh 2205 relative to a representation of the idealized model 2205b). Such an initial user alignment may be useful, e.g., in the early stages of a surgical examination, when only a small portion of the final captured mesh is available. One will appreciate that such user alignment may itself be used to guide future alignment attempts (e.g., with an updated model) by, e.g., restricting the search space of the particle filter or other pose alignment algorithm around the orientation provided by the user.

Where a landmark was identified at block 2250a, the system may determine if alignment is possible, or may at least be improved, using the landmark at block 2250b. For example, not all landmarks may be definitively located with a spatial location (e.g., a “smoke” or “biomass occlusion” landmark may occur at multiple locations during the examination). Similarly, some idealized models may not include landmarks recognized by the capturing system and vice versa. Where alignment is possible, or will at least benefit from the landmark correspondence, the system may attempt the alignment using the one or more landmarks as anchor(s) at block 2250c. For example, if a sufficient number of spatial landmarks have been recognized, the alignment may be “fully constrained” so that mesh 2205 may be directly scaled, rotated, or translated to accomplish the alignment.

### Example GUI Operations—Highlighting

Various embodiments enable reviewers to quickly and efficiently review examination data using a versatile interface, which may build upon various of the processing operations disclosed herein. Such a quick review may be valuable for procedural investigations (such as billing code verification), but may in fact be life-saving or life-enhancing when directed to identifying substantive surgical features (e.g., polyp removal, reaching a landmark, locating a polyp in preparation for laparoscopic surgery, etc.). As described herein, embodiments may facilitate such review by linking synthetic or “true to life” anatomy representations of internal body structures to relevant sequences of surgical data, such as endoscopic video. Landmarks in the data may be marked upon the anatomy representation to quickly facilitate reviewer identification as well as correlated with corresponding portions of the surgical data. This presentation facilitates an iterative high and low-level assessment by the reviewer as the reviewer may quickly traverse spatially and temporally separated portions of the data. The combination of 3D reconstruction algorithms disclosed herein, video to model structure mapping, and temporal/spatial scrolling, may together greatly improve the reviewer's assessment.

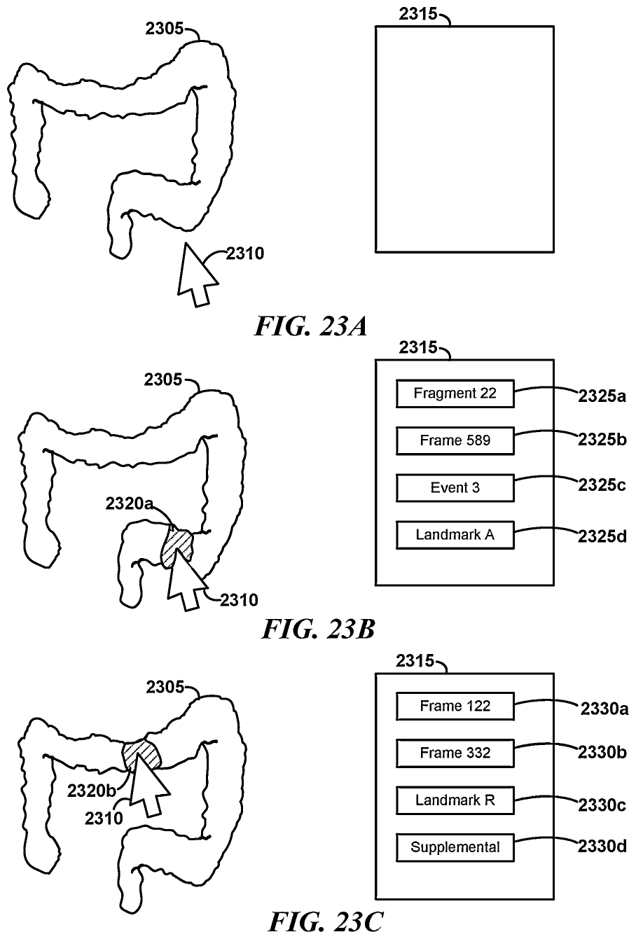

Specifically, in some embodiments, the GUI presented to the user may include elements as shown FIG. 23A (one will appreciate that the elements of FIG. 23A may appear in a GUI such as the GUI 1800 depicted in FIG. 18). In this example, a computer generated rendering of the model 2305 may be presented in the GUI to the user (e.g., in region 1805a). The user may rotate, translate, scale, etc., the model via a number of controls, such as movement of a mouse in combination with various keyboard commands or clicking of various of the mouse buttons. The present position of the mouse may be indicated by a cursor 2310. In this example, movement of the cursor 2310 over a specific portion of the model 2305 may precipitate a quick overview of data relevant to that region, e.g., by populating an item highlight pane 2315. As the cursor is not presently over the model in FIG. 23A, the panel 2315 is presently empty.

In contrast, in FIG. 23B the user has moved the mouse to a second position, such that the cursor 2310 now overlays a portion of the model 2305 in the model's current orientation. One will appreciate that the portion of the model 2305 beneath the cursor may be readily identified using any suitable ray intersection algorithm (for example taking the ray from a point corresponding to the cursor position in the plane of the viewer and extending the ray perpendicular to the plane out to infinity). A portion 2320a of the model 2305 corresponding to region overlaid by the cursor 2310 may now be identified to the viewer, e.g., via a billboard outline (a planar surface rendered before the viewer's virtual camera in the same plane as the viewer, with a texture selected to correspond to an two-dimensional outline around portion 2320a), by a change in texture of the mesh at portion 2320a, change in vertex shading of the mesh at portion 2320a, change in vertex edge rendering in the mesh at portion 2320a, etc. As the cursor may only overlay a small region of the model mesh, the portion 2320a may be determined, e.g., by extending the intersecting plane of the mesh to a fixed number of neighboring planes, by selecting vertices within a distance of the intersection point, vertices within a convex hull at the point of intersection, etc.

In addition to the highlighting of the region 2320a, the pane 2315 may be updated to provide indicia 2325a, 2325b, 2325c, 2325d indicating landmarks, events, fragments, etc. associated with the region 2320a. Where the cursor has been moved during playback, the population of pane 2315 may be temporally constrained, e.g., to only those events, landmarks, etc. relevant to the current playback time (e.g., wherein the related data was timestamped within a threshold distance of the current playback time or where the landmark is only spatial, without any temporal limitation). However, if playback is not ongoing or relevant to the GUI presentation, the pane 2315 may be populated with items spatially relevant to region 2320a regardless of their temporal character.

Selecting one of indicia 2325a, 2325b, 2325c, 2325c may present corresponding information. For example, selecting video frame indicia 2325b may present the visual image frame to the user or may begin playback from the time of that visual image's acquisition. Similarly, selecting an event indicia 2325c or a fragment indicia 2325a may present data associated with that fragment or event, such as the corresponding depth values, timestamp, etc. This may be useful for quickly debugging errors in the data capture or in the model. For clarity, where the region 2320a corresponds to a supplemented region or hole, in some embodiments fragments not used to generate the model 2305, but found to be relevant to the hole or supplemented region (e.g., based upon temporally neighboring fragments which were incorporated into the model), may likewise be provided. Selecting the landmark indicia 2325d may present the visual image frame or frames precipitating the landmark determination, as well as the landmark classification results. Selecting certain classes of landmarks (e.g., polyp detection) or items (e.g., operator bookmarks) may result in the presentation of new panes (e.g., diagnostic details regarding the polyp, a medical history, dictated notes associated with a bookmark, etc.). In some embodiments, the user may be able to edit these data values as, e.g., where the system made an incorrect landmark determination.

Thus, pane 2315 may provide indicia, e.g., indicia 2325a of the fragment or fragments used to generate the region 2320a. Similarly, the video frame or frames depicting portions of the region 2320a may also be provided, e.g., via indicia 2325b. Events may also be indicated, e.g., via indicia 2325c. Events may be bookmarks the user has created for a given region with a typed note, transcriptions or audio playback captured during a portion of the examination (e.g., a note dictated by the operator during the procedure), patient medical history appended by an assistant, etc. Previously identified and bookmarked landmarks may also be identified, e.g., via indicia 2325c (e.g., identification of a polyp, removal of a cancerous growth, application of a medication, etc.). In some embodiments, reimbursement billing codes associated with a landmark may also be presented in pane 2315 (e.g., recognition of a polyp or polyp removal landmark may be each associated with a corresponding billing code).

For clarity, FIG. 23C shows a similar adjustment when the cursor 2310 is moved to a third position, resulting in the highlighting of region 2320b (as well as the de-highlighting of region 2320a). Similarly, pane 2315 may now be updated to reflect the items relevant to region 2320b. Note that as the examination many have traversed the same spatial region on more than one occasion there may been multiple items, separated in time, but associated with the same spatial location. Thus, there may be multiple video frames 2330a, 2330b, corresponding, e.g., to a first passage through the region 2320b en route to a target location and then a second passage through region 2320b during extraction. Supplemental indicia 2330d may notify the reviewer that the region 2320b includes holes or supplemented data, while landmark indicia 2330c may notify the user of the presence of a new landmark.

While FIGS. 23A-C demonstrate embodiments where model selection precipitates panel population, one will appreciate that in some embodiments, the panel may be prepopulated, e.g., with all the known items, or those items related to a given moment in playback. Selection of the indicia in these instances with cursor 2310 may then result in highlighting of the corresponding portion of the model 2305. Such “reverse” indexing may be useful, for example, in embodiments where more than one capture device appeared in the patient's body during surgery (as when an assisting surgeon operated a peer device) and so it may be easier to review the dataset by beginning with the processed items and their temporal indications, rather than reviewing spatially by selecting portions of the model. With two capture devices, spatially separate events may occur at the same moment, and so the reverse indexing may more readily indicate the relationship by highlighting both relevant portions of the model.

Though the above examples discuss cursor control via a mouse, one will appreciate that a touchscreen, non-touch user interface, wheel-ball, augmented-reality input, etc. may each be equivalently used. During a surgery, the operator's hands may already be engaged with various devices and so voice commands, eye movements, etc. may be used to direct a cursor or make select model or item selections. Even assistants may avoid controlling a mouse in some situations, so as to maintain the sterile field.

### Example GUI Operations—Coordinated Playback—Visualization

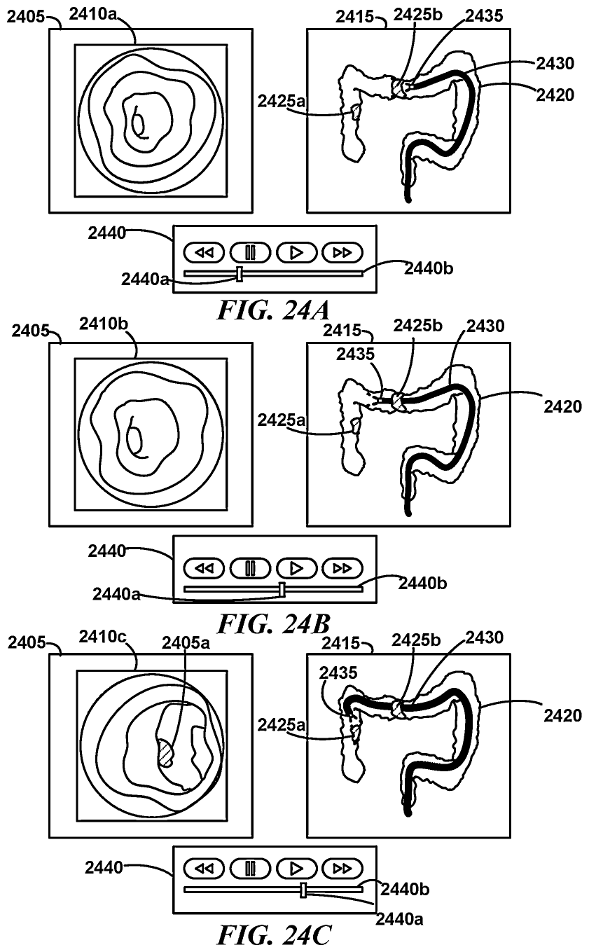

As mentioned above, various embodiments contemplate adjusting the presentation of data and adjustments to model rendering based upon the state of data playback. The GUI elements of FIG. 24A include a video image playback region 2405, rendered model region 2415, and playback controls region 2440. As indicated, playback controls region 2440 may include a timeline indicia 2440b and a current playback indicia 2440a (again, one will appreciate that the elements of FIG. 24A may appear in a GUI such as the GUI 1800 depicted in FIG. 18). In this example, the user may pause, play, rewind, or fast-forward the playback using the controls in playback controls region 2440. As playback advances, each of the current playback indicia 2440a, video image playback region 2405, and rendered model region 2415 may be updated. For example, the video image playback region 2405 may be updated to reflect the visual image 2410a corresponding to the current playback position. Similarly, rendered model region 2415 may be updated to reflect changes in the animated texturing of model 2420, the position of data capture position representation 2430, etc.

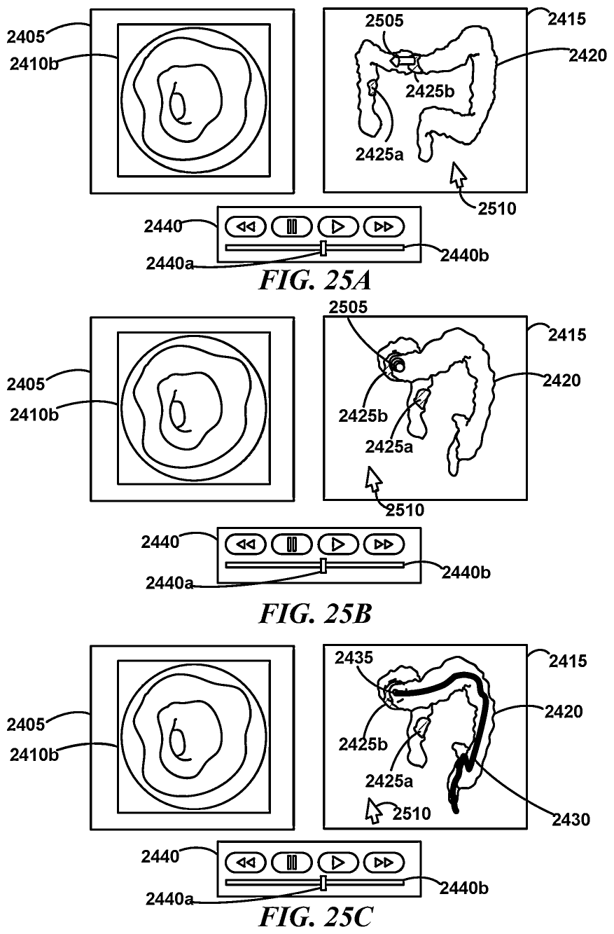

In this example, the position representation 2430 is shown as a computer rendered model of a colonoscope. Such a rendering may be appropriate, e.g., where the colonoscope's general path and point of entry are known. However, as discussed above, in some embodiments the position representation 2430 may be a three dimensional arrow, or other suitable indicia (as will be disclosed in greater detail below with reference to FIG. 25A), which may be more suitable for internal body structures imposing fewer limitations on the capture device's orientation. Rendering position representation 2430 with a computer generated model of the data capture device used to perform the actual data capture may have various benefits. For example, rendering the distal tip 2435 and bendable portion of the colonoscope provides the reviewer an easily interpretable understanding of the state of the surgical examination, the orientation precipitating the image 2410a, and spatial relation of the data capture device relative to regions of interest in the model, such as regions 2425b and 2425a. For example, region 2425b may correspond to a hole or supplemented portion of model 2420, while region 2425a may correspond to an event occurring during the surgery (e.g., identification of a polyp, a bookmark by the operator, a bleeding landmark detection, etc.). Such readily available topographic indications facilitate quick and efficient orientation of the reviewer to the large and multifaceted datasets, particularly where the dataset may also include information from the surgeon's console 155 (such as the state and configuration of the data capture device).

As shown in FIG. 24B, playback has advanced forward in time relative to FIG. 24A, as evidenced by the new position of current playback indicia 2440a relative to timeline indicia 2440b. Accordingly, the image playback region 2405 has been updated to reflect a new visual image 2410b corresponding to the new position in playback. Similarly, the placement of the position representation 2430 may be advanced as shown (one will appreciate that the distal tip's 2430 field of view has advanced in the model in parallel with the representation of new visual image 2410b).

At a yet further advanced time in playback after FIG. 24B, as shown in FIG. 24C, playback indicia 2440a is further advanced, image playback region 2405 is updated with a new image 2410c, and the position representation 2430 is further advanced. As shown, the rendering of the position representation 2430 may anticipate the behavior of the device being represented, here showing a bending of the bendable portion commensurate with the field of view capturing the corresponding portion of the model. Note that the cross-referencing between landmark data, fragment data, video visual image data, surgeon console data, etc., may facilitate complementary renderings between the different datasets. In the example shown here, image playback region 2405 has been overlaid with a translucent two-dimensional shape 2405a which may correspond, e.g., to the identification of a polyp landmark using a You Only Look Once (YOLO) neural network or an attention network. Highlighting such a region with, e.g., translucent two-dimensional shape 2405a may readily indicate the relation between the depicted image 2410c and the region 2425a in the model (e.g., rendering both with a same color or distinctive identifying icon). That is, following a landmark classification, the system may determine the corresponding depth values related to the relevant portion of the visual image (or the entire image) identified, e.g., by the YOLO network, and mark the corresponding portions of the model to be distinctively rendered so as to represent the relation.

To provide further clarity, FIG. 25A shows the substitution of an arrow position representation 2505 in place of the colonoscope model for position representation 2430. Also shown is a cursor 2510 (e.g., the same as cursor 2310). As indicated, in the depicted embodiment the arrow position representation 2505 is rendered last in the rendering pipeline, facilitating its placement over the model 2420 regardless of the model's orientation. This may facilitate easy interpretation of the current orientation corresponding to image 2410b and the state of playback, despite the varied orientation of the model. For example, movement of cursor 2510 while holding down a left or right mouse button from the cursor's 2510 position in FIG. 25A to the position of FIG. 25B may precipitate rotation of the model 2420 to the new orientation shown in FIG. 25B. Again, for clarity, FIG. 25C demonstrates rendering in this orientation for model 2420 but with the arrow position representation 2505 replaced again by the colonoscope model position representation 2430.

### Example GUI Operations—Coordinated Playback—Example Processes

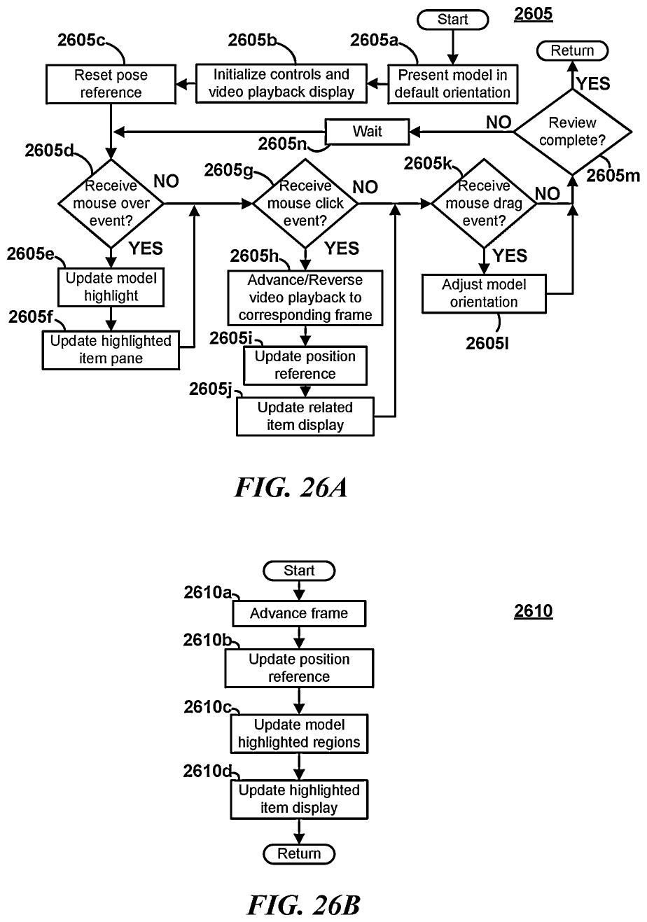

FIG. 26A is a flow diagram illustrating various operations in an example process 2605 for managing a GUI, such as a GUI of FIGS. 23A-C, 24A-C, and 25A-C (each of which may appear in interface 1800), as may be implemented in some embodiments. For example, at start up initialization of the GUI at block 2605a the system may present the model 2420 in a default orientation, initialize the controls 2440 at block 2605b (e.g., move current playback indicia 2440a to a leftmost position for the beginning of playback), and present the visual image for the first captured frame in the video image playback region 2405. Similarly, at block 2605c the system may reset the pose reference (e.g., move arrow position representation 2505 or colonoscope model position representation 2430, etc., to a position corresponding to the first image of playback).

Following this initialization, the system may enter a loop indicated by block 2605m, wherein the system waits at block 2605n until it receives a relevant input (one will appreciate that playback rendering may occur during this time, e.g., as discussed with respect to the process 2610 below). For example, if at block 2605d the system receives a mouse over event, the system may begin the operations described above with respect to FIGS. 23A-C, updating the model highlight (e.g., as discussed for region portion 2320a in FIG. 23B and region portion 2320b in FIG. 23C) at block 2605e and the highlighted item pane (e.g., pane 2315) at block 2605f.

Similarly, where a mouse click event is received in region 2415 and upon the model region 2420 at block 2605g, the system may advance or reverse the playback in region 2405 to the corresponding video frame at block 2605h, update the position reference (e.g., one of arrow position representation 2505 or colonoscope model position representation 2430) at block 2605i, and update the highlighted item pane 2315 at block 2605j. Finally, in this example, dragging of the mouse in region at block 2605k may be construed as a desire to rotate the model 2420 in pane 2415, e.g., as shown in FIGS. 25B and 25C, at block 26051.

During each iteration of playback (e.g., with the rendering of each successive visual image) the system may perform update playback process 2610 shown in FIG. 26B. At block 2610a, the system may update region 2405 to depict the visual image frame corresponding to the current playback time. Similarly, the system may adjust the pose reference (e.g., one of arrow position representation 2505 or colonoscope model position representation 2430) to reflect the new corresponding position of the capture device at block 2610b. At block 2610c, the system may highlight portions of the model, e.g., those corresponding to the field of view of the capture device at the current moment in playback relative to the model 2420. Thus, one will appreciate that there may be different model highlighting styles in process 2610 as compared to the user selected highlighting of FIGS. 23A-C. However, like the highlighting of FIGS. 23A-C, at block 2610d, the computer system may update the highlights pane 2315 to reflect new indicia related to the regions identified at block 2610c (e.g., landmarks or other structures detected by a YOLO network in the currently depicted visual image frame).

Again, while presented here separately to facilitate the reader's understanding, one will appreciate that one or more of the specific features disclosed herein, e.g., those with respect to FIGS. 13A-D, 22A, 22B, 23A, 23B, 23C, 24A, 24B, 24C, 25A, 25B, and 25C, may be applied alone or in combination in a single GUI interface, such as interface 1800.

### Example In-Review Landmark Detection

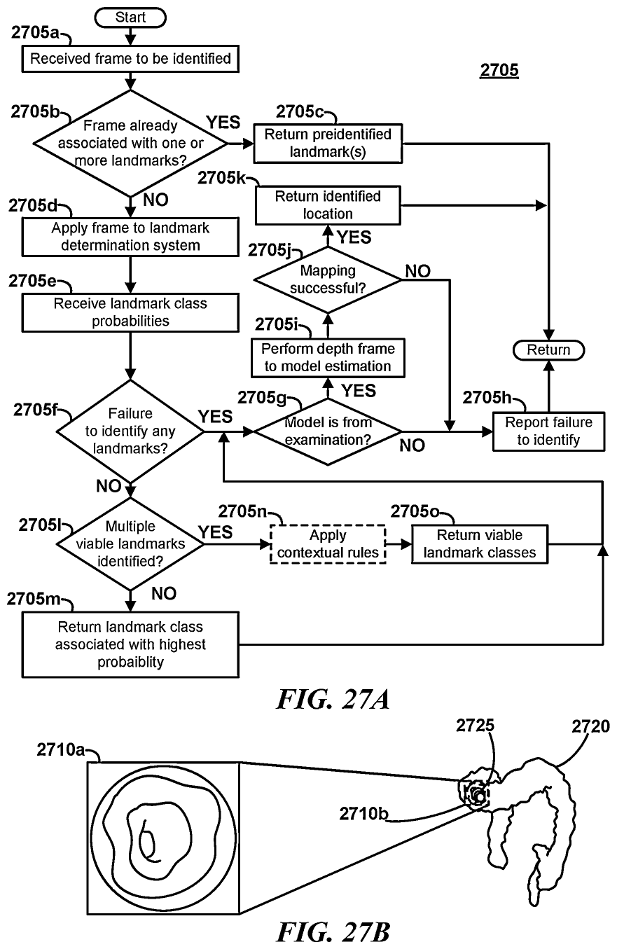

While various of the disclosed embodiments perform landmark detection prior to the model rendering, one will appreciate that computational complexity and the limited constraints of real-time operation may limit the frequency with which landmark detection may be performed. Accordingly, as shown in the process 2705 of FIG. 27A, landmark detection may be performed upon request during or following an operation in some embodiments. While process 2705 may be run as part of a playback interface, e.g., as discussed with respect FIGS. 24 and 25, process 2705 may also be run without a GUI (e.g., as part of an alert or navigation system).

At block 2705a, the system may receive a new visual image frame for landmark identification (e.g., as selected by a surgical assistant reviewing a recently captured image frame in the surgery). If a landmark was already determined to be associated with the visual image frame at block 2705b, the system may simply return the previously identified landmark at block 2705c (e.g., if a landmark was determined for a temporally nearby frame in the same fragment). However, if there is no landmark for the visual image frame, the system may apply the landmark determination system to the visual image frame at block 2705d to receive the various classification probabilities at block 2705e. If the results indicate that no landmark has been identified at block 2705f, e.g., if none of the classification probabilities exceed a threshold, if a corresponding uncertainty value is too high, if the highest probability is associated with a “not a landmark” class, etc., then at block 2705g the system may verify whether an available model is based upon a real-world data capture or is a virtual, idealized model. If the model was not generated from the current examination data, then the system may simply report failure at block 2705h (as the synthetic model is not itself indicative of events during the examination). However, if the model was created from examination fragments, then at block 2705i, the system may attempt to perform a depth frame to model estimation (e.g., using a particle filter, determined correspondences, or other localization methods). Application of such a localization algorithm may not be necessary where the correspondence between the visual image, depth frame, and position in the final model were previously recorded. If such a correspondence is available, then the operation of block 2705i may be straightforward, involving simply a look up to determine which portion of the model corresponds to the visual image. If the location in the model is successfully located at block 2705j, the relevant portion of the model may be returned at block 2705k with an indication that the landmark could not be identified. This response may be useful, e.g., where a surgical assistant or other team member is attempting to quickly locate a visual frame in the context of the preceding surgery. Absent landmark confirmation, the assistant may now be able to direct the surgeon back to the relevant portion of the internal body structure, so as to verify the anomaly appearing in the visual image. Thus, in some embodiments, and in contrast to the depicted example, where only a virtual, idealized model is available, the operations following a YES at block 2705g may always be performed (e.g., localizing with a particle filter) to help orient the visual image to the model for the user (indeed, this functionality may be provided to the user independently of a landmark classification request).

For example, as shown in FIG. 27B, the GUI may present an overlay 2710b upon the model 2720 indicating where the requested visual image 2710a appeared to correspond. A position reference 2725, such as an arrow, may also be provided to demonstrate the orientation of the capture device when capturing the image 2710a. Overlay 2710b may be a planar surface textured with the image 2710a at less than full opacity so as to allow the surgeon or assistant to recognize the correspondence between the model structure and the image structure. Thus, even absent a landmark, the user should be able to quickly identify where in the internal body structure the visual image was captured.

In some embodiments, the landmark classifier may be able to indicate the presence of multiple landmarks at block 27051. For example, the system may determine that image 2710a depicts both a known location landmark (e.g., the rectum) as well as a known procedure action landmark (e.g., retroflexion). If only a single landmark is detected, the landmark may be returned at block 2705m. However, where multiple landmarks were identified, then at blocks 2705n and 27050 the system may apply contextual rules to determine whether to return one or more of the landmarks as being appropriate. For example, as mentioned, some landmarks may only occur following other landmarks. Similarly, it may be impossible for some landmarks to have occurred before or after a certain time in the procedure (e.g., closure is unlikely to occur early in the surgery). Accordingly, premature recognition of a landmark may suggest that the landmark was improperly identified. However, as indicated by block 2705n's dashed lines, some embodiments forego the application of contextual rules and return all the identified landmark classes.

Following a successful identification of a landmark at block 2705f, in some embodiments the process may return. However, in the depicted example, the system may also quickly attempt to perform the previously described location determination starting at block 2705g. Attempting the location determination of block 2705g may be foregone where there is limited time or processing resources, e.g., where the particle filter localization is expected to be unacceptably taxing. However providing the user with both a landmark determination and a location relative to the model may greatly facilitate the user's situational awareness relative to the requested visual image.

### Computer System

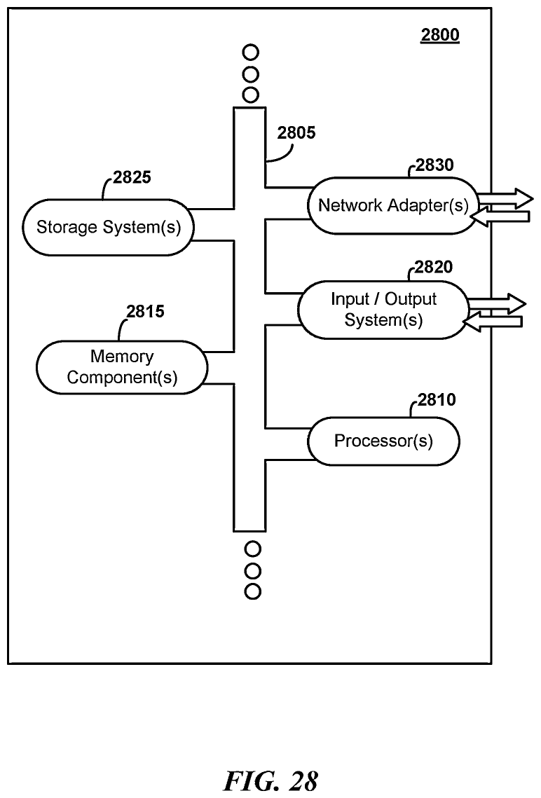

FIG. 28 is a block diagram of an example computer system as may be used in conjunction with some of the embodiments. The computing system 2800 may include an interconnect 2805, connecting several components, such as, e.g., one or more processors 2810, one or more memory components 2815, one or more input/output systems 2820, one or more storage systems 2825, one or more network adaptors 2830, etc. The interconnect 2805 may be, e.g., one or more bridges, traces, busses (e.g., an ISA, SCSI, PCI, 12C, Firewire bus, etc.), wires, adapters, or controllers.

The one or more processors 2810 may include, e.g., an Intel™ processor chip, a math coprocessor, a graphics processor, etc. The one or more memory components 2815 may include, e.g., a volatile memory (RAM, SRAM, DRAM, etc.), a non-volatile memory (EPROM, ROM, Flash memory, etc.), or similar devices. The one or more input/output devices 2820 may include, e.g., display devices, keyboards, pointing devices, touchscreen devices, etc. The one or more storage devices 2825 may include, e.g., cloud based storages, removable USB storage, disk drives, etc. In some systems memory components 2815 and storage devices 2825 may be the same components. Network adapters 2830 may include, e.g., wired network interfaces, wireless interfaces, Bluetooth™ adapters, line-of-sight interfaces, etc.

One will recognize that only some of the components, alternative components, or additional components than those depicted in FIG. 28 may be present in some embodiments. Similarly, the components may be combined or serve dual-purposes in some systems. The components may be implemented using special-purpose hardwired circuitry such as, for example, one or more ASICs, PLDs, FPGAS, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 2830. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 2815 and one or more storage devices 2825 may be computer-readable storage media. In some embodiments, the one or more memory components 2815 or one or more storage devices 2825 may store instructions, which may perform or cause to be performed various of the operations discussed herein. In some embodiments, the instructions stored in memory 2815 can be implemented as software and/or firmware. These instructions may be used to perform operations on the one or more processors 2810 to carry out processes described herein. In some embodiments, such instructions may be provided to the one or more processors 2810 by downloading the instructions from another system, e.g., via network adapter 2830.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only, and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

1-60. (canceled)

61. A computer system comprising:
at least one computer processor; and
at least one memory, the at least one memory comprising instructions configured to cause the computer system to perform a method, the method comprising:
causing a first graphical user interface element to be presented upon at least one display, the first graphical user interface element depicting a field of view of a surgical instrument within a patient interior body structure during a surgical procedure; and
causing a second graphical user interface element to be presented upon at least one display, the second graphical user interface element depicting a computer-generated three-dimensional model of at least a portion of the patient interior body structure, the three-dimensional model generated, at least in part, based upon images acquired with the surgical instrument.

62. The computer system of claim 61, wherein the method further comprises:
generating the three-dimensional model of the at least a portion of the patient interior body structure, wherein generating the three-dimensional model comprises:
receiving a plurality of visual images, the plurality of visual images depicting fields of view of the surgical instrument within the patient interior body structure;
determining a plurality of depth frames corresponding to the plurality of visual images using at least one machine learning architecture;
assembling the plurality of depth frames into a plurality of fragments; and
integrating the fragments to create the three-dimensional model of the at least a portion of the patient interior body structure.

63. The computer system of claim 62, wherein,
each of the fragments comprises a corresponding keyframe of a plurality of keyframes, and wherein,
integrating the fragments comprises determining a graph pose network based upon the plurality of keyframes.

64. The computer system of claim 63, wherein determining the graph pose network comprises:
for each of the keyframes, generating a plurality of sets of features for a plurality of visual images captured with the surgical instrument;
determining a plurality of poses for the plurality of visual images based upon correspondences between the sets of features; and
determining reachability between two or more of the keyframes based, at least in part, upon the poses of the two or more of the keyframes.

65. The computer system of claim 61, wherein the second graphical user interface element includes a position representation of the surgical instrument, the position representation of the surgical instrument oriented in agreement with the field of view of the surgical instrument depicted in the first graphical user interface element.

66. The computer system of claim 61, wherein the method further comprises:
detecting a hole in the computer-generated three-dimensional model; and
generating a rendering of the computer-generated model with an indication of the hole.

67. The computer system of claim 61, wherein the method further comprises:
detecting at least one landmark in a visual image associated with a portion of the computer-generated three-dimensional model;
determining an alignment of the computer-generated three-dimensional model with a synthetic model, using the at least one landmark; and
presenting the computer-generated three-dimensional model in the second graphical user interface element in accordance with the determined alignment.

68. A non-transitory computer-readable medium comprising instructions configured to cause at least one computer system to perform a method, the method comprising:
causing a first graphical user interface element to be presented upon at least one display, the first graphical user interface element depicting a field of view of a surgical instrument within a patient interior body structure during a surgical procedure; and
causing a second graphical user interface element to be presented upon at least one display, the second graphical user interface element depicting a computer-generated three-dimensional model of at least a portion of the patient interior body structure, the three-dimensional model generated, at least in part, based upon images acquired with the surgical instrument.

69. The non-transitory computer-readable medium of claim 68, wherein the method further comprises:
generating the three-dimensional model of the at least a portion of the patient interior body structure, wherein generating the three-dimensional model comprises:
receiving a plurality of visual images, the plurality of visual images depicting fields of view of the surgical instrument within the patient interior body structure;
determining a plurality of depth frames corresponding to the plurality of visual images using at least one machine learning architecture;
assembling the plurality of depth frames into a plurality of fragments; and
integrating the fragments to create the three-dimensional model of the at least a portion of the patient interior body structure.

70. The non-transitory computer-readable medium of claim 69, wherein,
each of the fragments comprises a corresponding keyframe of a plurality of keyframes, and wherein,
integrating the fragments comprises determining a graph pose network based upon the plurality of keyframes.

71. The non-transitory computer-readable medium of claim 70, wherein determining the graph pose network comprises:
for each of the keyframes, generating a plurality of sets of features for a plurality of visual images captured with the surgical instrument;
determining a plurality of poses for the plurality of visual images based upon correspondences between the sets of features; and
determining reachability between two or more of the keyframes based, at least in part, upon the poses of the two or more of the keyframes.

72. The non-transitory computer-readable medium of claim 68, wherein the second graphical user interface element includes a position representation of the surgical instrument, the position representation of the surgical instrument oriented in agreement with the field of view of the surgical instrument depicted in the first graphical user interface element.

73. The non-transitory computer-readable medium of claim 68, wherein the method further comprises:
detecting a hole in the computer-generated three-dimensional model; and
generating a rendering of the computer-generated model with an indication of the hole.

74. The non-transitory computer-readable medium of claim 68, wherein the method further comprises:
detecting at least one landmark in a visual image associated with a portion of the computer-generated three-dimensional model;
determining an alignment of the computer-generated three-dimensional model with a synthetic model, using the at least one landmark; and
presenting the computer-generated three-dimensional model in the second graphical user interface element in accordance with the determined alignment.

75. A computer-implemented method, the method comprising:
causing a first graphical user interface element to be presented upon at least one display, the first graphical user interface element depicting a field of view of a surgical instrument within a patient interior body structure during a surgical procedure; and
causing a second graphical user interface element to be presented upon at least one display, the second graphical user interface element depicting a computer-generated three-dimensional model of at least a portion of the patient interior body structure, the three-dimensional model generated, at least in part, based upon images acquired with the surgical instrument.

76. The computer-implemented method of claim 75, wherein the method further comprises:
generating the three-dimensional model of the at least a portion of the patient interior body structure, wherein generating the three-dimensional model comprises:
receiving a plurality of visual images, the plurality of visual images depicting fields of view of the surgical instrument within the patient interior body structure;
determining a plurality of depth frames corresponding to the plurality of visual images using at least one machine learning architecture;
assembling the plurality of depth frames into a plurality of fragments; and
integrating the fragments to create the three-dimensional model of the at least a portion of the patient interior body structure.

77. The computer-implemented method of claim 76, wherein,
each of the fragments comprises a corresponding keyframe of a plurality of keyframes, and wherein,
integrating the fragments comprises determining a graph pose network based upon the plurality of keyframes.

78. The computer-implemented method of claim 77, wherein determining the graph pose network comprises:
for each of the keyframes, generating a plurality of sets of features for a plurality of visual images captured with the surgical instrument;
determining a plurality of poses for the plurality of visual images based upon correspondences between the sets of features; and
determining reachability between two or more of the keyframes based, at least in part, upon the poses of the two or more of the keyframes.

79. The computer-implemented method of claim 75, wherein the second graphical user interface element includes a position representation of the surgical instrument, the position representation of the surgical instrument oriented in agreement with the field of view of the surgical instrument depicted in the first graphical user interface element.

80. The computer-implemented method of claim 75, wherein the method further comprises:
detecting at least one landmark in a visual image associated with a portion of the computer-generated three-dimensional model;
determining an alignment of the computer-generated three-dimensional model with a synthetic model, using the at least one landmark; and
presenting the computer-generated three-dimensional model in the second graphical user interface element in accordance with the determined alignment.
